(19) United States
(12) Patent Application Publication
(10) Pub. No.: US 2018/0222868 A1
(43) Pub. Date: Aug. 9, 2018

(54) Indazole Derivatives as Modulators of TNF Activity

(71) Applicant: UCB Biopharma SPRL, Brussels (BE)

(72) Inventors: Prafulkumar Tulshibhai Chovatia, Abingdon, Oxfordshire (GB); Martin Clive Hutchings, Slough, Berkshire (GB); Boris Kroeplien, Slough, Berkshire (GB); James Thomas Reuberson, Slough, Berkshire (GB)

(21) Appl. No.: 15/580,054

(22) PCT Filed: Jun. 7, 2016
(86) PCT No.: PCT/EP2016/062901

§ 371 Date: Dec. 6, 2017

(30) Foreign Application Priority Data
Jun. 8, 2015 (GB) 1509885.8

(51) Int. Cl. C07D 231/56; A61K 31/416; C07D 403/04; A61K 31/506; C07D 403/14; C07D 413/14

(52) U.S. Cl. CPC C07D 231/56; A61K 31/416; C07D 413/14; A61K 31/506; C07D 403/14; C07D 403/04

(57) ABSTRACT

A series of substituted indazole derivatives, being potent modulators of human TNFa activity, are accordingly of benefit in the treatment and/or prevention of various human ailments, including autoimmune and inflammatory disorders; neurological and neurodegenerative disorders; pain and nociceptive disorders; cardiovascular disorders; metabolic disorders; ocular disorders; and oncological disorders.

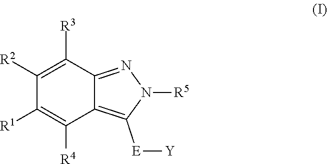

The present invention relates to a class of fused pyrazole derivatives, and to their use in therapy. More particularly, this invention is concerned with pharmacologically active substituted indazole derivatives. These compounds are modulators of the signalling of TNFα, and are accordingly of benefit as pharmaceutical agents, especially in the treatment of adverse inflammatory and autoimmune disorders, neurological and neurodegenerative disorders, pain and nociceptive disorders, cardiovascular disorders, metabolic disorders, ocular disorders, and oncological disorders.

TNFα is the prototypical member of the Tumour Necrosis Factor (TNF) superfamily of proteins that share a primary function of regulating cell survival and cell death. One structural feature common to all known members of the TNF superfamily is the formation of trimeric complexes that bind to, and activate, specific TNF superfamily receptors. By way of example, TNFα exists in soluble and transmembrane forms and signals through two receptors, known as TNFR1 and TNFR2, with distinct functional endpoints.

Various products capable of modulating TNFα activity are already commercially available. All are approved for the treatment of inflammatory and autoimmune disorders such as rheumatoid arthritis and Crohn's disease. All currently approved products are macromolecular and act by inhibiting the binding of human TNFα to its receptor. Typical macromolecular TNFα inhibitors include anti-TNFα antibodies; and soluble TNFα receptor fusion proteins. Examples of commercially available anti-TNFα antibodies include fully human antibodies such as adalimumab (Humira®) and golimumab (Simponi®), chimeric antibodies such as infliximab (Remicade®), and pegylated Fab′ fragments such as certolizumab pegol (Cimzia®). An example of a commercially available soluble TNFα receptor fusion protein is etanercept (Enbrel®).

TNF superfamily members, including TNFα itself, are implicated in a variety of physiological and pathological functions that are believed to play a part in a range of conditions of significant medical importance (see, for example, M. G. Tansey & D. E. Szymkowski, Drug Discovery Today, 2009, 14, 1082-1088; and F. S. Carneiro et al., J. Sexual Medicine, 2010, 7, 3823-3834).

The compounds in accordance with the present invention, being potent modulators of human TNFα activity, are therefore beneficial in the treatment and/or prevention of various human ailments. These include autoimmune and inflammatory disorders; neurological and neurodegenerative disorders; pain and nociceptive disorders; cardiovascular disorders; metabolic disorders; ocular disorders; and oncological disorders.

In addition, the compounds in accordance with the present invention may be beneficial as pharmacological standards for use in the development of new biological tests and in the search for new pharmacological agents. Thus, in one embodiment, the compounds of this invention may be useful as radioligands in assays for detecting pharmacologically active compounds. In an alternative embodiment, certain compounds of this invention may be useful for coupling to a fluorophore to provide fluorescent conjugates that can be utilised in assays (e.g. a fluorescence polarisation assay) for detecting pharmacologically active compounds.

WO 2013/186229, WO 2014/009295 and WO 2014/009296 describe fused imidazole derivatives which are modulators of human TNFα activity.

None of the prior art available to date, however, discloses or suggests the precise structural class of indazole derivatives as provided by the present invention.

The present invention provides a compound of formula (I) or an N-oxide thereof, or a pharmaceutically acceptable salt thereof:

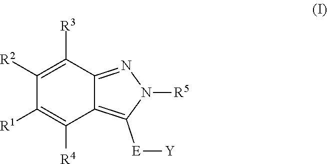

wherein

E represents a covalent bond; or E represents —O—, —S—, —S(O)—, —S(O)₂— or —N(R⁶)—; or E represents an optionally substituted straight or branched C₁₋₄ alkylene chain;

Y represents Y¹ or Y²;

Y¹ represents C₃₋₇ cycloalkyl, aryl, C₃₋₇ heterocycloalkyl or heteroaryl, any of which groups may be optionally substituted by one or more substituents;

Y² represents a group of formula (Ya), (Yb), (Yc), (Yd), (Ye) or (Yf):

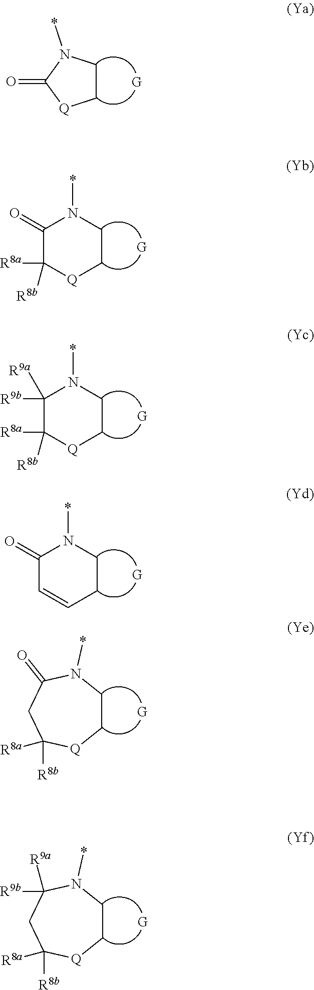

the asterisk (*) represents the point of attachment to the remainder of the molecule;

Q represents —O—, —S—, —S(O)—, —S(O)₂—, —S(O)(NR⁶)—, —N(R⁶)—, —C(O)— or —C(R^{7a})(R^{7b})—;

G represents the residue of an optionally substituted benzene ring; or an optionally substituted five-membered heteroaromatic ring selected from furyl, thienyl, pyrrolyl, pyrazolyl, oxazolyl, isoxazolyl, thiazolyl, isothiazolyl, imidazolyl, oxadiazolyl, thiadiazolyl and triazolyl; or an optionally substituted six-membered heteroaromatic ring selected from pyridinyl, pyridazinyl, pyrimidinyl, pyrazinyl and triazinyl;

R¹, R², R³ and R⁴ independently represent hydrogen, halogen, cyano, nitro, hydroxy, trifluoromethyl, trifluoromethoxy, —OR^{a}, —SR^{a}, —SOR^{a}, —SO₂R^{a}, —SF₅, —NR^{b}R^{c}, —NR^{c}COR^{d}, —NR^{c}CO₂R^{d}, —NHCONR^{b}R^{c}, —NR^{c}SO₂R^{e}, —N(SO₂R^{e})₂, —NHSO₂NR^{b}R^{c}, —COR^{d}, —CO₂R^{d}, —CONR^{b}R^{c}, —CON(OR^{a})R^{b}, —SO₂NR^{b}R^{c} or —SO(NR^{b})R^{d}; or C₁₋₆ alkyl, C₂₋₆ alkenyl, C₂₋₆ alkynyl, C₃₋₇ cycloalkyl, C₄₋₇ cycloalkenyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, C₃₋₇ heterocycloalkenyl, C₄₋₉ heterobicycloalkyl, heteroaryl, heteroaryl(C₁₋₆)alkyl, (C₃₋₇)heterocycloalkyl(C₁₋₆)alkyl-aryl-, heteroaryl(C₃₋₇)heterocycloalkyl-, (C₃₋₇)cycloalkyl-heteroaryl-, (C₃₋₇)cycloalkyl(C₁₋₆)alkyl-heteroaryl-, (C₄₋₇)cycloalkenyl-heteroaryl-, (C₄₋₉)bicycloalkyl-heteroaryl-, (C₃₋₇)heterocycloalkyl-heteroaryl-, (C₃₋₇)heterocycloalkyl(C₁₋₆)alkyl-heteroaryl-, (C₃₋₇)heterocycloalkenyl-heteroaryl-, (C₄₋₉)heterobicycloalkyl-heteroaryl- or (C₄₋₉)spiroheterocycloalkyl-heteroaryl-, any of which groups may be optionally substituted by one or more substituents;

R⁵ represents C₁₋₆ alkyl, optionally substituted by fluoro, hydroxy, C₁₋₆ alkoxy, amino, C₁₋₆ alkylamino or di(C₁₋₆)alkylamino;

R⁶ represents hydrogen or C₁₋₆ alkyl;

R^{7a} and R^{7b} independently represent hydrogen or C₁₋₆ alkyl;

R^{8a} and R^{8b} independently represent hydrogen, halogen or C₁₋₆ alkyl; or

R^{8a} and R^{8b}, when taken together with the carbon atom to which they are both attached, represent C₃₋₇ cycloalkyl or C₃₋₇ heterocycloalkyl, either of which groups may be optionally substituted by one or more substituents; or

R^{7a} and R^{8a}, when taken together with the two intervening carbon atoms, represent C₃₋₇ cycloalkyl or C₃₋₇ heterocycloalkyl, either of which groups may be optionally substituted by one or more substituents;

R^{9a} and R^{9b} independently represent hydrogen or C₁₋₆ alkyl; or

R^{9a} and R^{9b}, when taken together with the carbon atom to which they are both attached, represent C₃₋₇ cycloalkyl or C₃₋₇ heterocycloalkyl, either of which groups may be optionally substituted by one or more substituents;

R^{a} represents C₁₋₆ alkyl, aryl, aryl(C₁₋₆)alkyl, heteroaryl or heteroaryl(C₁₋₆)alkyl, any of which groups may be optionally substituted by one or more substituents;

R^{b} and R^{c} independently represent hydrogen or trifluoromethyl; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents; or

R^{b} and R^{c}, when taken together with the nitrogen atom to which they are both attached, represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents;

R^{d} represents hydrogen; or C₁₋₆ alkyl, C₃₋₇ cycloalkyl, aryl, C₃₋₇ heterocycloalkyl or heteroaryl, any of which groups may be optionally substituted by one or more substituents; and

R^{e} represents C₁₋₆ alkyl, aryl or heteroaryl, any of which groups may be optionally substituted by one or more substituents.

The present invention also provides a compound of formula (I) as defined above or an N-oxide thereof, or a pharmaceutically acceptable salt thereof, for use in therapy.

The present invention also provides a compound of formula (I) as defined above or an N-oxide thereof, or a pharmaceutically acceptable salt thereof, for use in the treatment and/or prevention of disorders for which the administration of a modulator of TNFα function is indicated.

In another aspect, the present invention provides a compound of formula (I) as defined above or an N-oxide thereof, or a pharmaceutically acceptable salt thereof, for use in the treatment and/or prevention of an inflammatory or autoimmune disorder, a neurological or neurodegenerative disorder, pain or a nociceptive disorder, a cardiovascular disorder, a metabolic disorder, an ocular disorder, or an oncological disorder.

The present invention also provides a method for the treatment and/or prevention of disorders for which the administration of a modulator of TNFα function is indicated which comprises administering to a patient in need of such treatment an effective amount of a compound of formula (I) as defined above or an N-oxide thereof, or a pharmaceutically acceptable salt thereof.

In another aspect, the present invention provides a method for the treatment and/or prevention of an inflammatory or autoimmune disorder, a neurological or neuro-degenerative disorder, pain or a nociceptive disorder, a cardiovascular disorder, a metabolic disorder, an ocular disorder, or an oncological disorder, which comprises administering to a patient in need of such treatment an effective amount of a compound of formula (I) as defined above or an N-oxide thereof, or a pharmaceutically acceptable salt thereof.

Where any of the groups in the compounds of formula (I) above is stated to be optionally substituted, this group may be unsubstituted, or substituted by one or more substituents. Typically, such groups will be unsubstituted, or substituted by one or two substituents.

For use in medicine, the salts of the compounds of formula (I) will be pharmaceutically acceptable salts. Other salts may, however, be useful in the preparation of the compounds of use in the invention or of their pharmaceutically acceptable salts. Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002.

The present invention includes within its scope solvates of the compounds of formula (I) above. Such solvates may be formed with common organic solvents or water.

The present invention also includes within its scope co-crystals of the compounds of formula (I) above. The technical term “co-crystal” is used to describe the situation where neutral molecular components are present within a crystalline compound in a definite stoichiometric ratio. The preparation of pharmaceutical co-crystals enables modifications to be made to the crystalline form of an active pharmaceutical ingredient, which in turn can alter its physicochemical properties without compromising its intended biological activity (see Pharmaceutical Salts and Co-crystals, ed. J. Wouters & L. Quere, RSC Publishing, 2012).

Suitable alkyl groups which may be present on the compounds of use in the invention include straight-chained and branched C₁₋₆ alkyl groups, for example C₁₋₄ alkyl groups. Typical examples include methyl and ethyl groups, and straight-chained or branched propyl, butyl and pentyl groups. Particular alkyl groups include methyl, ethyl, n-propyl, isopropyl, n-butyl, sec-butyl, isobutyl, tert-butyl, 2,2-dimethylpropyl and 3-methylbutyl. Derived expressions such as “C₁₋₆ alkoxy”, “C₁₋₆ alkylthio”, “C₁₋₆ alkylsulphonyl” and “C₁₋₆ alkylamino” are to be construed accordingly.

The expression “C₁₋₄ alkylene chain” refers to a divalent straight or branched alkylene chain containing 1 to 4 carbon atoms. Typical examples include methylene, ethylene, methylmethylene, ethylmethylene and dimethylmethylene.

Suitable C₂₋₆ alkenyl groups include vinyl and allyl.

Suitable C₂₋₆ alkynyl groups include ethynyl, propargyl and butynyl.

The term “C₃₋₇ cycloalkyl” as used herein refers to monovalent groups of 3 to 7 carbon atoms derived from a saturated monocyclic hydrocarbon, and may comprise benzo-fused analogues thereof. Suitable C₃₋₇ cycloalkyl groups include cyclopropyl, cyclobutyl, benzocyclobutenyl, cyclopentyl, indanyl, cyclohexyl and cycloheptyl.

The term “C₄₋₇ cycloalkenyl” as used herein refers to monovalent groups of 4 to 7 carbon atoms derived from a partially unsaturated monocyclic hydrocarbon. Suitable C₄₋₇ cycloalkenyl groups include cyclobutenyl, cyclopentenyl, cyclohexenyl and cycloheptenyl.

The term “C₄₋₉ bicycloalkyl” as used herein refers to monovalent groups of 4 to 9 carbon atoms derived from a saturated bicyclic hydrocarbon. Typical bicycloalkyl groups include bicyclo[3.1.0]hexanyl, bicyclo[4.1.0]heptanyl and bicyclo[2.2.2]octanyl.

The term “aryl” as used herein refers to monovalent carbocyclic aromatic groups derived from a single aromatic ring or multiple condensed aromatic rings. Suitable aryl groups include phenyl and naphthyl, preferably phenyl.

Suitable aryl(C₁₋₆)alkyl groups include benzyl, phenylethyl, phenylpropyl and naphthylmethyl.

The term “C₃₋₇ heterocycloalkyl” as used herein refers to saturated monocyclic rings containing 3 to 7 carbon atoms and at least one heteroatom selected from oxygen, sulphur and nitrogen, and may comprise benzo-fused analogues thereof. Suitable heterocycloalkyl groups include oxetanyl, azetidinyl, tetrahydrofuranyl, dihydrobenzo-furanyl, dihydrobenzothienyl, pyrrolidinyl, indolinyl, isoindolinyl, oxazolidinyl, thiazolidinyl, isothiazolidinyl, imidazolidinyl, tetrahydropyranyl, chromanyl, tetrahydro-thiopyranyl, piperidinyl, 1,2,3,4-tetrahydroquinolinyl, 1,2,3,4-tetrahydroisoquinolinyl, piperazinyl, 1,2,3,4-tetrahydroquinoxalinyl, hexahydro-[1,2,5]thiadiazolo[2,3-a]pyrazinyl, homopiperazinyl, morpholinyl, benzoxazinyl, thiomorpholinyl, azepanyl, oxazepanyl, diazepanyl, thiadiazepanyl and azocanyl.

The term “C₃₋₇ heterocycloalkenyl” as used herein refers to monounsaturated or polyunsaturated monocyclic rings containing 3 to 7 carbon atoms and at least one heteroatom selected from oxygen, sulphur and nitrogen, and may comprise benzo-fused analogues thereof. Suitable heterocycloalkenyl groups include thiazolinyl, isothiazolinyl, imidazolinyl, dihydropyranyl, dihydrothiopyranyl and 1,2,3,6-tetrahydropyridinyl.

The term “C₄₋₉ heterobicycloalkyl” as used herein corresponds to C₄₋₉ bicycloalkyl wherein one or more of the carbon atoms have been replaced by one or more heteroatoms selected from oxygen, sulphur and nitrogen. Typical heterobicycloalkyl groups include 3-azabicyclo[3.1.0]hexanyl, 2-oxa-5-azabicyclo[2.2.1]heptanyl, 6-azabicyclo[3.2.0]heptanyl, 3-azabicyclo[3.1.1]heptanyl, 3-azabicyclo[4.1.0]heptanyl, 2-oxabicyclo[2.2.2]octanyl, quinuclidinyl, 2-oxa-5-azabicyclo[2.2.2]octanyl, 3-azabicyclo[3.2.1]octanyl, 8-azabicyclo-[3.2.1]octanyl, 3-oxa-8-azabicyclo[3.2.1]octanyl, 3,8-diazabicyclo[3.2.1]octanyl, 3,6-diazabicyclo[3.2.2]nonanyl, 3-oxa-7-azabicyclo[3.3.1]nonanyl and 3,9-diazabicyclo-[4.2.1]nonanyl.

The term “C₄₋₉ spiroheterocycloalkyl” as used herein refers to saturated bicyclic ring systems containing 4 to 9 carbon atoms and at least one heteroatom selected from oxygen, sulphur and nitrogen, in which the two rings are linked by a common atom. Suitable spiroheterocycloalkyl groups include 5-azaspiro[2.3]hexanyl, 5-azaspiro[2.4]-heptanyl, 2-azaspiro[3.3]heptanyl, 2-oxa-6-azaspiro[3.3]heptanyl, 2-oxa-6-azaspiro[3.4]-octanyl, 2-oxa-6-azaspiro[3.5]nonanyl, 7-oxa-2-azaspiro[3.5]nonanyl, 2-oxa-7-azaspiro-[3.5]nonanyl and 2,4,8-triazaspiro[4.5]decanyl.

The term “heteroaryl” as used herein refers to monovalent aromatic groups containing at least 5 atoms derived from a single ring or multiple condensed rings, wherein one or more carbon atoms have been replaced by one or more heteroatoms selected from oxygen, sulphur and nitrogen. Suitable heteroaryl groups include furyl, benzofuryl, dibenzofuryl, thienyl, benzothienyl, thieno[2,3-c]pyrazolyl, thieno[3,4-b][1,4]dioxinyl, dibenzothienyl, pyrrolyl, indolyl, pyrrolo[2,3-b]pyridinyl, pyrrolo[3,2-c]pyridinyl, pyrrolo[3,4-b]pyridinyl, pyrazolyl, pyrazolo[1,5-a]pyridinyl, pyrazolo[3,4-d]pyrimidinyl, indazolyl, 4,5,6,7-tetrahydroindazolyl, oxazolyl, benzoxazolyl, isoxazolyl, thiazolyl, benzothiazolyl, isothiazolyl, imidazolyl, benzimidazolyl, imidazo[2,1-b]thiazolyl, imidazo[1,2-a]pyridinyl, imidazo[4,5-b]pyridinyl, purinyl, imidazo[1,2-a]pyrimidinyl, imidazo[1,2-a]pyrazinyl, oxadiazolyl, thiadiazolyl, triazolyl, [1,2,4]triazolo[1,5-a]-pyrimidinyl, benzotriazolyl, tetrazolyl, pyridinyl, quinolinyl, isoquinolinyl, naphthyridinyl, pyridazinyl, cinnolinyl, phthalazinyl, pyrimidinyl, quinazolinyl, pyrazinyl, quinoxalinyl, pteridinyl, triazinyl and chromenyl groups.

The term “halogen” as used herein is intended to include fluorine, chlorine, bromine and iodine atoms, typically fluorine, chlorine or bromine.

Where the compounds of formula (I) have one or more asymmetric centres, they may accordingly exist as enantiomers. Where the compounds of use in the invention possess two or more asymmetric centres, they may additionally exist as diastereomers.

The invention is to be understood to extend to the use of all such enantiomers and diastereomers, and to mixtures thereof in any proportion, including racemates. Formula (I) and the formulae depicted hereinafter are intended to represent all individual stereoisomers and all possible mixtures thereof, unless stated or shown otherwise. In addition, compounds of formula (I) may exist as tautomers, for example keto (CH₂C═O)↔enol (CH═CHOH) tautomers or amide (NHC═O)↔hydroxyimine (N═COH) tautomers. Formula (I) and the formulae depicted hereinafter are intended to represent all individual tautomers and all possible mixtures thereof, unless stated or shown otherwise.

It is to be understood that each individual atom present in formula (I), or in the formulae depicted hereinafter, may in fact be present in the form of any of its naturally occurring isotopes, with the most abundant isotope(s) being preferred. Thus, by way of example, each individual hydrogen atom present in formula (I), or in the formulae depicted hereinafter, may be present as a ¹H, ²H (deuterium) or ³H (tritium) atom, preferably ¹H. Similarly, by way of example, each individual carbon atom present in formula (I), or in the formulae depicted hereinafter, may be present as a ¹²C, ¹³C or ¹⁴C atom, preferably ¹²C.

In a particular aspect, the present invention provides a compound of formula (I) as depicted above or an N-oxide thereof, or a pharmaceutically acceptable salt thereof, wherein

R¹ represents halogen or cyano; or C₁₋₆ alkyl, C₂₋₆ alkenyl, C₂₋₆ alkynyl, C₃₋₇ cycloalkyl, C₄₋₇ cycloalkenyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, C₃₋₇ heterocycloalkenyl, C₄₋₉ heterobicycloalkyl, heteroaryl, heteroaryl(C₁₋₆)alkyl, (C₃₋₇)heterocycloalkyl(C₁₋₆)alkyl-aryl-, heteroaryl(C₃₋₇)heterocycloalkyl-, (C₃₋₇)cycloalkyl-heteroaryl-, (C₃₋₇)cycloalkyl-(C₁₋₆)alkyl-heteroaryl-, (C₄₋₇)cycloalkenyl-heteroaryl-, (C₄₋₉)bicycloalkyl-heteroaryl-, (C₃₋₇)heterocycloalkyl-heteroaryl-, (C₃₋₇)heterocycloalkyl(C₁₋₆)alkyl-heteroaryl-, (C₃₋₇)heterocycloalkenyl-heteroaryl-, (C₄₋₉)heterobicycloalkyl-heteroaryl- or (C₄₋₉)spiroheterocycloalkyl-heteroaryl-, any of which groups may be optionally substituted by one or more substituents; and

E, Y, R², R³, R⁴ and R⁵ are as defined above.

Where the compounds in accordance with the invention comprise an optionally substituted straight or branched alkylene chain, typical values thereof include methylene (—CH₂—), (methyl)methylene, ethylene (—CH₂CH₂—), (ethyl)methylene, (dimethyl)-methylene, (methyl)ethylene, propylene (—CH₂CH₂CH₂—), (propyl)methylene and (dimethyl)ethylene, any of which chains may be optionally substituted by one or more substituents. Suitably, such chains are unsubstituted, monosubstituted or disubstituted. Typically, such chains are unsubstituted or monosubstituted. In one embodiment, such chains are unsubstituted. In another embodiment, such chains are monosubstituted. In a further embodiment, such chains are disubstituted.

Examples of typical substituents on the alkylene chain which may be present in a compound in accordance with the invention include halogen, cyano, trifluoromethyl, oxo, hydroxy, C₁₋₆ alkoxy, carboxy(C₁₋₆)alkoxy, trifluoromethoxy, amino, C₁₋₆ alkylamino, di(C₁₋₆)alkylamino, C₂₋₆ alkylcarbonylamino, carboxy, benzyloxycarbonyl, tetrazolyl, aminocarbonyl, C₁₋₆ alkylaminocarbonyl and di(C₁₋₆)alkylaminocarbonyl.

Specific examples of suitable substituents on the alkylene chain which may be present in a compound in accordance with the invention include fluoro, cyano, trifluoromethyl, hydroxy, methoxy, carboxymethoxy, amino, acetylamino, carboxy, benzyloxycarbonyl and tetrazolyl.

In a first embodiment, E represents a covalent bond, whereby the integer Y is attached directly to the pyrazole ring.

In a second embodiment, E represents —O—, —S—, —S(O)—, —S(O)₂— or —N(R⁶)—. In a first aspect of that embodiment, E represents —O—. In a second aspect of that embodiment, E represents —S—. In a third aspect of that embodiment, E represents —S(O)—. In a fourth aspect of that embodiment, E represents —S(O)₂—. In a fifth aspect of that embodiment, E represents —N(R⁶)—.

In a third embodiment, E represents an optionally substituted straight or branched C₁₋₄ alkylene chain. In a first aspect of that embodiment, E represents an optionally substituted methylene (—CH₂—) linkage. In a second aspect of that embodiment, E represents an optionally substituted (methyl)methylene linkage. In a third aspect of that embodiment, E represents an optionally substituted (ethyl)methylene linkage.

Generally, E represents a covalent bond; or E represents —N(R⁶)—; or E represents an optionally substituted straight or branched C₁₋₄ alkylene chain.

Typically, E represents —N(R⁶)—; or E represents an optionally substituted straight or branched C₁₋₄ alkylene chain.

Suitably, E represents a covalent bond; or E represents —N(R⁶)—; or E represents methylene (—CH₂—), (methyl)methylene or (ethyl)methylene, any of which groups may be optionally substituted by one or more substituents.

Appositely, E represents —N(R⁶)—, or optionally substituted methylene.

Selected examples of typical substituents on the linkage represented by E include halogen, trifluoromethyl, hydroxy, C₁₋₆ alkoxy, carboxy(C₁₋₆)alkoxy, trifluoromethoxy, amino, C₁₋₆ alkylamino, di(C₁₋₆)alkylamino, C₂₋₆ alkylcarbonylamino, carboxy, benzyloxycarbonyl and tetrazolyl.

Specific examples of typical substituents on the linkage represented by E include fluoro, trifluoromethyl, hydroxy, methoxy, carboxymethoxy, trifluoromethoxy, amino, methylamino, dimethylamino, acetylamino, carboxy, benzyloxycarbonyl and tetrazolyl.

A particular example of a typical substituent on E is hydroxy.

Typical values of E include —N(R⁶)—, —CH₂—, —CH(OH)—, —CH(OCH₃)—, —CH(OCH₂CO₂H)—, —CH(NH₂)—, —CH(NHCOCH₃)—, —CH(CO₂H)—, —CH(CO₂benzyl)-, —CH(CH₃)—, —C(CH₃)(OH)— and —CH(CH₂CH₃)—; or E may represent a covalent bond.

Typical values of E include —N(R⁶)—, —CH₂— and —CH(OH)—.

Suitable values of E include —CH₂— and —CH(OH)—.

In one embodiment, E represents —N(R⁶)—.

In another embodiment, E represents —CH₂—.

In a further embodiment, E represents —CH(OH)—.

In another embodiment, E represents —CH(OCH₃)—.

In another embodiment, E represents —CH(NH₂)—.

In an additional embodiment, E represents —CH(CH₃)—. In a particular aspect of that embodiment, the —CH(CH₃)— linkage represented by E is in the (S) stereochemical configuration.

In a further embodiment, E represents —C(CH₃)(OH)—.

In a first embodiment, Y represents Y¹. In a second embodiment, Y represents Y².

Generally, Y¹ represents C₃₋₇ cycloalkyl, aryl or heteroaryl, any of which groups may be optionally substituted by one or more substituents.

Typically, Y¹ represents aryl or heteroaryl, either of which groups may be optionally substituted by one or more substituents.

In a first embodiment, Y¹ represents optionally substituted C₃₋₇ cycloalkyl. In one aspect of that embodiment, Y¹ represents unsubstituted C₃₋₇ cycloalkyl. In another aspect of that embodiment, Y¹ represents monosubstituted C₃₋₇ cycloalkyl. In a further aspect of that embodiment, Y¹ represents disubstituted C₃₋₇ cycloalkyl.

In a second embodiment, Y¹ represents optionally substituted aryl. In one aspect of that embodiment, Y¹ represents unsubstituted aryl. In another aspect of that embodiment, Y¹ represents monosubstituted aryl. In a further aspect of that embodiment, Y¹ represents disubstituted aryl.

In a third embodiment, Y¹ represents optionally substituted C₃₋₇ heterocycloalkyl. In one aspect of that embodiment, Y¹ represents unsubstituted C₃₋₇ heterocycloalkyl. In another aspect of that embodiment, Y¹ represents monosubstituted C₃₋₇ heterocycloalkyl. In a further aspect of that embodiment, Y¹ represents disubstituted C₃₋₇ heterocycloalkyl.

In a fourth embodiment, Y¹ represents optionally substituted heteroaryl. In one aspect of that embodiment, Y¹ represents unsubstituted heteroaryl. In another aspect of that embodiment, Y¹ represents monosubstituted heteroaryl. In a further aspect of that embodiment, Y¹ represents disubstituted heteroaryl.

Suitably, Y¹ represents benzocyclobutenyl, phenyl, thienyl, thiazolyl or pyridinyl, any of which groups may be optionally substituted by one or more substituents.

Appropriately, Y¹ represents phenyl, thienyl or thiazolyl, any of which groups may be optionally substituted by one or more substituents.

Appositely, Y¹ represents phenyl, which may be optionally substituted by one or more substituents.

Examples of optional substituents which may be present on the moiety Y¹ include one, two or three substituents independently selected from halogen, cyano, nitro, C₁₋₆ alkyl, trifluoromethyl, hydroxy, C₁₋₆ alkoxy, difluoromethoxy, trifluoromethoxy, C₁₋₆ alkylthio, C₁₋₆ alkylsulfinyl, C₁₋₆ alkylsulfonyl, (C₁₋₆)alkylsulfonyloxy, amino, C₁₋₆ alkyl-amino, di(C₁₋₆)alkylamino, arylamino, C₂₋₆ alkylcarbonylamino, C₁₋₆ alkylsulfonylamino, formyl, C₂₋₆ alkylcarbonyl, C₃₋₆ cycloalkylcarbonyl, C₃₋₆ heterocycloalkylcarbonyl, carboxy, C₂₋₆ alkoxycarbonyl, aminocarbonyl, C₁₋₆ alkylaminocarbonyl, di(C₁₋₆)alkyl-aminocarbonyl, aminosulfonyl, C₁₋₆ alkylaminosulfonyl and di(C₁₋₆)alkylaminosulfonyl.

Typical examples of optional substituents on the moiety Y¹ include halogen, cyano and difluoromethoxy.

Suitable examples of optional substituents on the moiety Y¹ include difluoro-methoxy.

Examples of particular substituents on the moiety Y¹ include fluoro, chloro, bromo, cyano, nitro, methyl, isopropyl, trifluoromethyl, hydroxy, methoxy, difluoromethoxy, trifluoromethoxy, methylthio, methylsulfinyl, methylsulfonyl, methylsulfonyloxy, amino, methylamino, tert-butylamino, dimethylamino, phenylamino, acetylamino, methyl-sulfonylamino, formyl, acetyl, cyclopropylcarbonyl, azetidinylcarbonyl, pyrrolidinyl-carbonyl, piperidinylcarbonyl, piperazinylcarbonyl, morpholinylcarbonyl, carboxy, methoxycarbonyl, aminocarbonyl, methylaminocarbonyl, dimethylaminocarbonyl, aminosulfonyl, methylaminosulfonyl and dimethylaminosulfonyl.

Typical examples of particular substituents on the moiety Y¹ include fluoro, chloro, cyano and difluoromethoxy.

Suitable examples of particular substituents on the moiety Y¹ include difluoro-methoxy.

Typical values of Y¹ include benzocyclobutenyl, phenyl, fluorophenyl (including 2-fluorophenyl, 3-fluorophenyl and 4-fluorophenyl), chlorophenyl (including 2-chloro-phenyl, 3-chlorophenyl and 4-chlorophenyl), difluorophenyl (including 2,6-difluoro-phenyl), (chloro)(fluoro)phenyl (including 5-chloro-2-fluorophenyl and 2-chloro-5-fluorophenyl), dichlorophenyl (including 2,5-dichlorophenyl and 2,6-dichlorophenyl), methylphenyl (including 4-methylphenyl), dimethylphenyl (including 2,5-dimethylphenyl and 2,6-dimethylphenyl), (trifluoromethyl)phenyl[including 2-(trifluoromethyl)phenyl], (chloro)(trifluoromethyl)phenyl[including 5-chloro-2-(trifluoromethyl)phenyl], (methyl)-(trifluoromethyl)phenyl[including 2-methyl-5-(trifluoromethyl)phenyl], bis(trifluoro-methyl)phenyl[including 2,5-bis(trifluoromethyl)phenyl], methoxyphenyl (including 2-methoxyphenyl), (difluoromethoxy)phenyl[including 2-(difluoromethoxy)phenyl and 3-(difluoromethoxy)phenyl], (difluoromethoxy)(fluoro)phenyl[including 2-(difluoro-methoxy)-5-fluorophenyl and 2-(difluoromethoxy)-6-fluorophenyl], (chloro)(difluoro-methoxy)phenyl[including 5-chloro-2-(difluoromethoxy)phenyl and 6-chloro-2-(difluoromethoxy)phenyl], (cyano)(difluoromethoxy)phenyl[including 6-cyano-2-(difluoromethoxy)phenyl], (trifluoromethoxy)phenyl[including 2-(trifluoromethoxy)-phenyl], methylsulfonyloxyphenyl, (amino)(chloro)phenyl (including 5-amino-2-chloro-phenyl), methylthienyl (including 3-methylthien-2-yl), methylthiazolyl (including 2-methyl-1,3-thiazol-4-yl), (chloro)(methyl)thiazolyl (including 5-chloro-2-methyl-1,3-thiazol-4-yl), dimethylthiazolyl (including 2,4-dimethyl-1,3-thiazol-5-yl) and pyridinyl (including pyridin-3-yl and pyridin-4-yl).

Selected values of Y¹ include dichlorophenyl, dimethylphenyl, (difluoromethoxy)-phenyl, (difluoromethoxy)(fluoro)phenyl, methylsulfonyloxyphenyl, methylthienyl and dimethylthiazolyl.

A specific value of Y¹ is (difluoromethoxy)phenyl.

In one embodiment, Y¹ represents 2,5-dichlorophenyl.

In another embodiment, Y¹ represents 2,5-dimethylphenyl.

In a particular embodiment, Y¹ represents 2-(difluoromethoxy)phenyl.

In another embodiment, Y¹ represents (difluoromethoxy)(fluoro)phenyl.

In another embodiment, Y¹ represents 3-methylthien-2-yl.

In another embodiment, Y¹ represents 2,4-dimethyl-1,3-thiazol-5-yl.

Typically, Q represents —O—, —S—, —S(O)— or —C(R^{7a})(R^{7b})—.

Suitably, Q represents —O— or —C(R^{7a})(R^{7b})—.

In a first embodiment, Q represents —O—. In a second embodiment, Q represents —S—. In a third embodiment, Q represents —S(O)—. In a fourth embodiment, Q represents —S(O)₂—. In a fifth embodiment, Q represents —S(O)(NR⁶)—. In a sixth embodiment, Q represents —N(R⁶)—. In a seventh embodiment, Q represents —C(O)—. In an eighth embodiment, Q represents —C(R^{7a})(R^{7b})—.

In the compounds of the invention, the moiety G is defined as representing the residue of an optionally substituted benzene ring, or an optionally substituted five-membered or six-membered heteroaromatic ring as specified above. From this it is to be understood that the variable G, when taken together with the two carbon atoms of the ring to which the G-containing ring is fused, represents an optionally substituted benzene ring, or an optionally substituted five-membered or six-membered heteroaromatic ring as specified above.

In a first embodiment, the moiety G in the compounds of the invention represents the residue of an optionally substituted benzene ring.

In a second embodiment, the moiety G in the compounds of the invention represents the residue of an optionally substituted five-membered heteroaromatic ring selected from furyl, thienyl, pyrrolyl, pyrazolyl, oxazolyl, isoxazolyl, thiazolyl, isothiazolyl, imidazolyl, oxadiazolyl, thiadiazolyl, triazolyl and tetrazolyl.

In a third embodiment, the moiety G in the compounds of the invention represents the residue of an optionally substituted six-membered heteroaromatic ring selected from pyridinyl, pyridazinyl, pyrimidinyl, pyrazinyl and triazinyl.

Generally, G represents the residue of an optionally substituted benzene ring, or an optionally substituted six-membered heteroaromatic ring as specified above.

Suitably, G represents the residue of an optionally substituted benzene ring; or an optionally substituted six-membered heteroaromatic ring selected from pyridinyl and pyrimidinyl.

The aromatic or heteroaromatic ring of which the moiety G is the residue may be unsubstituted, or may be substituted, where possible, by one or more substituents, generally by one, two or three substituents, typically by one or two substituents. In one embodiment, this ring is unsubstituted. In another embodiment, this ring is monosubstituted. In a further embodiment, this ring is disubstituted. In a still further embodiment, this ring is trisubstituted.

Typical examples of optional substituents on the aromatic or heteroaromatic ring of which the moiety G is the residue include halogen, cyano, C₁₋₆ alkyl, fluoromethyl, difluoromethyl, trifluoromethyl, hydroxy, hydroxy(C₁₋₆)alkyl, C₁₋₆ alkoxy, difluoro-methoxy, trifluoromethoxy, pentafluorothio, C₁₋₆ alkylthio, C₁₋₆ alkylsulphinyl, C₁₋₆ alkylsulphonyl, amino, amino(C₁₋₆)alkyl, C₁₋₆ alkylamino, di(C₁₋₆)alkylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulphonyl, C₁₋₆ alkylaminosulphonyl, di(C₁₋₆)alkyl-aminosulphonyl, (C₁₋₆)alkylsulphoximinyl and [(C₁₋₆)alkyl][N—(C₁₋₆)alkyl]sulphoximinyl, hydroxy(C₁₋₆)alkylaminocarbonyl, (C₁₋₆)alkoxy(C₁₋₆)alkylaminocarbonyl, (C₃₋₇)cycloalkyl-aminocarbonyl, heteroaryl(C₁₋₆)alkylaminocarbonyl, hydroxy(C₃₋₇)heterocycloalkyl, (C₁₋₆)alkoxy(C₃₋₇)heterocycloalkyl, (C₃₋₇)heterocycloalkylcarbonyl, hydroxy(C₃₋₇)-heterocycloalkylcarbonyl, oxo(C₃₋₇)heterocycloalkylcarbonyl, (C₁₋₆)alkylsulphonyl-(C₃₋₇)heterocycloalkylcarbonyl and (C₂₋₆)alkoxycarbonyl(C₃₋₇)heterocycloalkylcarbonyl.

Suitable examples of optional substituents on the aromatic or heteroaromatic ring of which the moiety G is the residue include halogen.

Typical examples of particular substituents on the aromatic or heteroaromatic ring of which the moiety G is the residue include fluoro, chloro, bromo, cyano, methyl, fluoromethyl, difluoromethyl, trifluoromethyl, hydroxy, hydroxymethyl, hydroxyethyl, hydroxyisopropyl, methoxy, difluoromethoxy, trifluoromethoxy, pentafluorothio, methylthio, methylsulphinyl, methylsulphonyl, amino, aminomethyl, methylamino, dimethylamino, formyl, acetyl, carboxy, methoxycarbonyl, ethoxycarbonyl, tert-butoxycarbonyl, aminocarbonyl, methylaminocarbonyl, dimethylaminocarbonyl, aminosulphonyl, methylaminosulphonyl, dimethylaminosulphonyl, methylsulphoximinyl and (methyl)(N-methyl)sulphoximinyl, ethylaminocarbonyl, isopropylaminocarbonyl, hydroxyethylaminocarbonyl, hydroxyisopropylaminocarbonyl, 1-hydroxy-2-methylprop-2-ylaminocarbonyl, methoxyethylaminocarbonyl, cyclopropylaminocarbonyl, oxazolylmethylaminocarbonyl, hydroxyoxetanyl, methoxyoxetanyl, piperazinylcarbonyl, hydroxypyrrolidinylcarbonyl, oxopiperazinylcarbonyl, methylsulphonylazetidinylcarbonyl and tert-butoxycarbonylpiperazinylcarbonyl.

Suitable examples of particular substituents on the aromatic or heteroaromatic ring of which the moiety G is the residue include fluoro.

Particular values of Y² include the groups of formula (Ya-1), (Ya-2), (Ya-3), (Yb-1), (Yb-2), (Yb-3), (Yb-4), (Yb-5), (Yb-6), (Yb-7), (Yc-1) and (Yd-1):

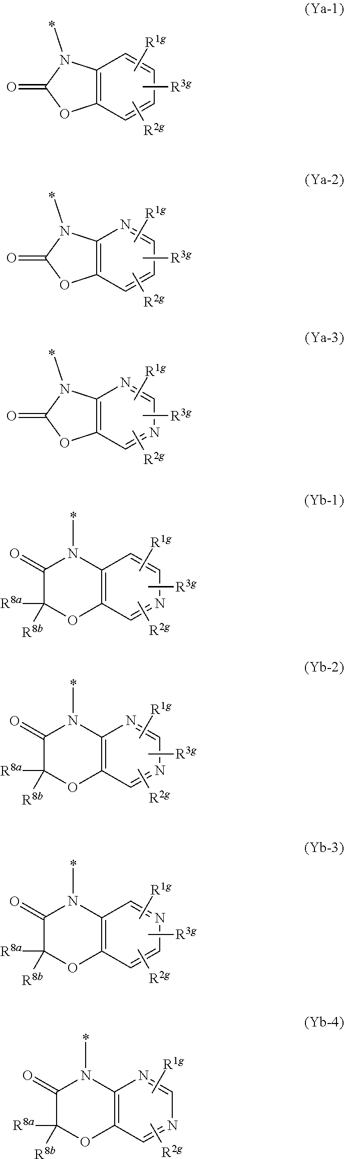

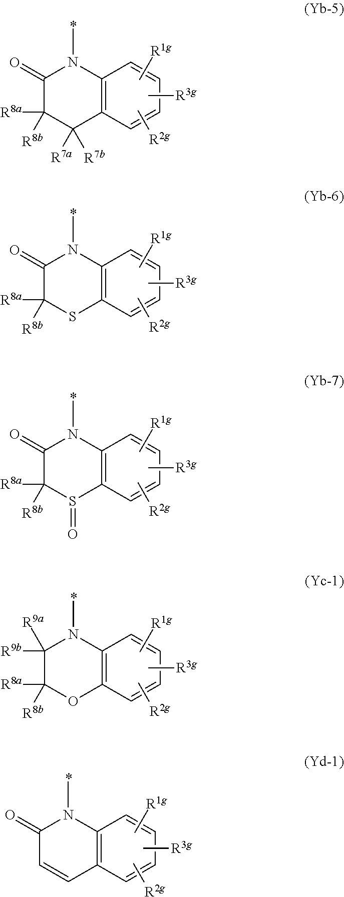

wherein

the asterisk (*) represents the point of attachment to the remainder of the molecule;

R^{1g} represents hydrogen, halogen, cyano, C₁₋₆ alkyl, fluoromethyl, difluoromethyl, trifluoromethyl, hydroxy, hydroxy(C₁₋₆)alkyl, C₁₋₆ alkoxy, difluoromethoxy, trifluoro-methoxy, pentafluorothio, C₁₋₆ alkylthio, C₁₋₆ alkylsulphinyl, C₁₋₆ alkylsulphonyl, amino, amino(C₁₋₆)alkyl, C₁₋₆ alkylamino, di(C₁₋₆)alkylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkyl-aminocarbonyl, hydroxy(C₁₋₆)alkylaminocarbonyl, (C₁₋₆)alkoxy(C₁₋₆)alkylaminocarbonyl, (C₃₋₇)cycloalkylaminocarbonyl, heteroaryl(C₁₋₆)alkylaminocarbonyl, aminosulphonyl, C₁₋₆ alkylaminosulphonyl, di(C₁₋₆)alkylaminosulphonyl, (C₁₋₆)alkylsulphoximinyl, [(C₁₋₆)alkyl][N—(C₁₋₆)alkyl]sulphoximinyl, hydroxy(C₃₋₇)heterocycloalkyl, (C₁₋₆)alkoxy-(C₃₋₇)heterocycloalkyl, (C₃₋₇)heterocycloalkylcarbonyl, hydroxy(C₃₋₇)heterocycloalkyl-carbonyl, oxo(C₃₋₇)heterocycloalkylcarbonyl, (C₁₋₆)alkylsulphonyl(C₃₋₇)heterocycloalkyl-carbonyl or (C₂₋₆)alkoxycarbonyl(C₃₋₇)heterocycloalkylcarbonyl;

R^{2g} and R^{3g} independently represent hydrogen or halogen; and

R^{7a}, R^{7b}, R^{8a}, R^{8b}, R^{9a} and R^{9b} are as defined above.

Suitable values of Y² include the groups of formula (Ya-1), (Ya-2), (Ya-3), (Yb-1), (Yb-2), (Yb-3), (Yb-4), (Yb-5), (Yc-1) and (Yd-1) as depicted above.

Appositely, Y² represents a group of formula (Yb-1) as depicted above.

Appositely, R^{1g} represents hydrogen, halogen, cyano, C₁₋₆ alkyl, fluoromethyl, difluoromethyl, trifluoromethyl, hydroxy, hydroxy(C₁₋₆)alkyl, C₁₋₆ alkoxy, difluoro-methoxy, trifluoromethoxy, pentafluorothio, C₁₋₆ alkylthio, C₁₋₆ alkylsulphinyl, C₁₋₆ alkylsulphonyl, amino, amino(C₁₋₆)alkyl, C₁₋₆ alkylamino, di(C₁₋₆)alkylamino, formyl, C₂₋₆ alkylcarbonyl, carboxy, C₂₋₆ alkoxycarbonyl, aminocarbonyl, C₁₋₆ alkylamino-carbonyl, di(C₁₋₆)alkylaminocarbonyl, aminosulphonyl, C₁₋₆ alkylaminosulphonyl, di(C₁₋₆)alkylaminosulphonyl, (C₁₋₆)alkylsulphoximinyl or [(C₁₋₆)alkyl][N—(C₁₋₆)alkyl]-sulphoximinyl.

Suitably, R^{1g} represents hydrogen or halogen.

Typical values of R^{1g} include hydrogen, fluoro, chloro, bromo, cyano, methyl, fluoromethyl, difluoromethyl, trifluoromethyl, hydroxy, hydroxymethyl, hydroxyethyl, hydroxyisopropyl, methoxy, difluoromethoxy, trifluoromethoxy, pentafluorothio, methylthio, methylsulphinyl, methylsulphonyl, amino, aminomethyl, methylamino, dimethylamino, formyl, acetyl, carboxy, methoxycarbonyl, ethoxycarbonyl, tert-butoxycarbonyl, aminocarbonyl, methylaminocarbonyl, dimethylaminocarbonyl, aminosulphonyl, methylaminosulphonyl, dimethylaminosulphonyl, methylsulphoximinyl and (methyl)(N-methyl)sulphoximinyl.

Illustrative values of R^{1g} include hydrogen and fluoro.

In a first embodiment, R^{2g} represents hydrogen. In a second embodiment, R^{2g} represents halogen. In one aspect of that embodiment, R^{2g} especially represents fluoro. In another aspect of that embodiment, R^{2g} represents chloro.

In a first embodiment, R^{3g} represents hydrogen. In a second embodiment, R^{3g} represents halogen, especially fluoro.

Suitably, R¹, R², R³ and R⁴ independently represent hydrogen, halogen, cyano, trifluoromethyl or —CO₂R^{d}; or C₁₋₆ alkyl, C₂₋₆ alkynyl, aryl, C₃₋₇ heterocycloalkyl, C₃₋₇ heterocycloalkenyl, heteroaryl, (C₃₋₇)heterocycloalkyl(C₁₋₆)alkyl-aryl-, heteroaryl-(C₃₋₇)heterocycloalkyl-, (C₃₋₇)cycloalkyl-heteroaryl-, (C₃₋₇)cycloalkyl(C₁₋₆)alkyl-heteroaryl-, (C₄₋₇)cycloalkenyl-heteroaryl-, (C₄₋₉)bicycloalkyl-heteroaryl-, (C₃₋₇)heterocycloalkyl-heteroaryl-, (C₃₋₇)heterocycloalkyl(C₁₋₆)alkyl-heteroaryl-, (C₃₋₇)heterocycloalkenyl-heteroaryl-, (C₄₋₉)heterobicycloalkyl-heteroaryl- or (C₄₋₉)spiroheterocycloalkyl-heteroaryl-, any of which groups may be optionally substituted by one or more substituents.

Examples of optional substituents which may be present on R¹, R², R³ or R⁴ include one, two or three substituents independently selected from halogen, halo-(C₁₋₆)alkyl, cyano, cyano(C₁₋₆)alkyl, nitro, nitro(C₁₋₆)alkyl, C₁₋₆ alkyl, difluoromethyl, trifluoromethyl, difluoroethyl, trifluoroethyl, C₂₋₆ alkenyl, hydroxy, hydroxy(C₁₋₆)alkyl, C₁₋₆ alkoxy, difluoromethoxy, trifluoromethoxy, trifluoroethoxy, carboxy(C₃₋₇)cycloalkyl-oxy, C₁₋₃ alkylenedioxy, C₁₋₆ alkoxy(C₁₋₆)alkyl, pentafluorothio, C₁₋₆ alkylthio, C₁₋₆ alkylsulphinyl, C₁₋₆ alkylsulphonyl, (C₁₋₆)alkylsulphonyl(C₁₋₆)alkyl, oxo, amino, amino-(C₁₋₆)alkyl, C₁₋₆ alkylamino, di(C₁₋₆)alkylamino, hydroxy(C₁₋₆)alkylamino, C₁₋₆ alkoxy-amino, (C₁₋₆)alkoxy(C₁₋₆)alkylamino, [(C₁₋₆)alkoxy](hydroxy)(C₁₋₆)alkylamino, [(C₁₋₆)alkylthio](hydroxy)(C₁₋₆)alkylamino, N—[(C₁₋₆)alkyl]-N-[hydroxy(C₁₋₆)alkyl]amino, di(C₁₋₆)alkylamino(C₁₋₆)alkylamino, N-[di(C₁₋₆)alkylamino(C₁₋₆)alkyl]-N-[hydroxy(C₁₋₆)-alkyl]amino, hydroxy(C₁₋₆)alkyl(C₃₋₇)cycloalkylamino, (hydroxy)[(C₃₋₇)cycloalkyl(C₁₋₆)-alkyl]amino, (C₃₋₇)heterocycloalkyl(C₁₋₆)alkylamino, oxo(C₃₋₇)heterocycloalkyl(C₁₋₆)alkyl-amino, (C₁₋₆)alkylheteroarylamino, heteroaryl(C₁₋₆)alkylamino, (C₁₋₆)alkylheteroaryl(C₁₋₆)-alkylamino, C₂₋₆ alkylcarbonylamino, N—[(C₁₋₆)alkyl]-N—[(C₂₋₆)alkylcarbonyl]amino, (C₂₋₆)-alkylcarbonylamino(C₁₋₆)alkyl, C₃₋₆ alkenylcarbonylamino, bis[(C₃₋₆)alkenylcarbonyl]-amino, N—[(C₁₋₆)alkyl]-N—[(C₃₋₇)cycloalkylcarbonyl]amino, C₂₋₆ alkoxycarbonylamino, C₂₋₆ alkoxycarbonyl(C₁₋₆)alkylamino, C₁₋₆ alkylaminocarbonylamino, C₁₋₆ alkylsulphonyl-amino, N—[(C₁₋₆)alkyl]-N—[(C₁₋₆)alkylsulphonyl]amino, bis[(C₁₋₆)alkylsulphonyl]amino, N—[(C₁₋₆)alkyl]-N-[carboxy(C₁₋₆)alkyl]amino, carboxy(C₃₋₇)cycloalkylamino, carboxy-(C₃₋₇)cycloalkyl(C₁₋₆)alkylamino, formyl, C₂₋₆ alkylcarbonyl, (C₃₋₇)cycloalkylcarbonyl, phenylcarbonyl, (C₂₋₆)alkylcarbonyloxy(C₁₋₆)alkyl, carboxy, carboxy(C₁₋₆)alkyl, C₂₋₆ alkoxycarbonyl, C₂₋₆ alkoxycarbonyl(C₁₋₆)alkyl, morpholinyl(C₁₋₆)alkoxycarbonyl, C₂₋₆ alkoxycarbonylmethylidenyl, a carboxylic acid isostere or prodrug moiety Ω, —(C₁₋₆)alkyl-Ω, aminocarbonyl, C₁₋₆ alkylaminocarbonyl, hydroxy(C₁₋₆)alkylamino-carbonyl, di(C₁₋₆)alkylaminocarbonyl, aminocarbonyl(C₁₋₆)alkyl, aminosulphonyl, di(C₁₋₆)alkylaminosulphonyl, (C₁₋₆)alkylsulphoximinyl, trifluoromethylsulphoximinyl, [(C₁₋₆)alkyl][N—(C₁₋₆)alkyl]sulphoximinyl, [(C₁₋₆)alkyl][N-carboxy(C₁₋₆)alkyl]-sulphoximinyl, [N—(C₂₋₆)alkoxycarbonyl(C₁₋₆)alkyl][(C₁₋₆)alkyl]sulphoximinyl, (C₃₋₇)cycloalkylsulphoximinyl and N-[di(C₁₋₆)alkylsulfoxo]iminyl.

By the expression “carboxylic acid isostere or prodrug moiety” is meant any functional group, structurally distinct from a carboxylic acid moiety, that will be recognised by a biological system as being similar to, and thus capable of mimicking, a carboxylic acid moiety, or will be readily convertible by a biological system in vivo into a carboxylic acid moiety. A synopsis of some common carboxylic acid isosteres is presented by N. A. Meanwell in J. Med. Chem., 2011, 54, 2529-2591 (cf. in particular FIGS. 25 and 26). An alternative carboxylic acid isostere is described by N Pemberton et al. in ACS Med. Chem. Lett., 2012, 3, 574-578. Typical examples of suitable carboxylic acid isostere or prodrug moieties represented by Ω include the functional groups of formula (i) to (xliii):

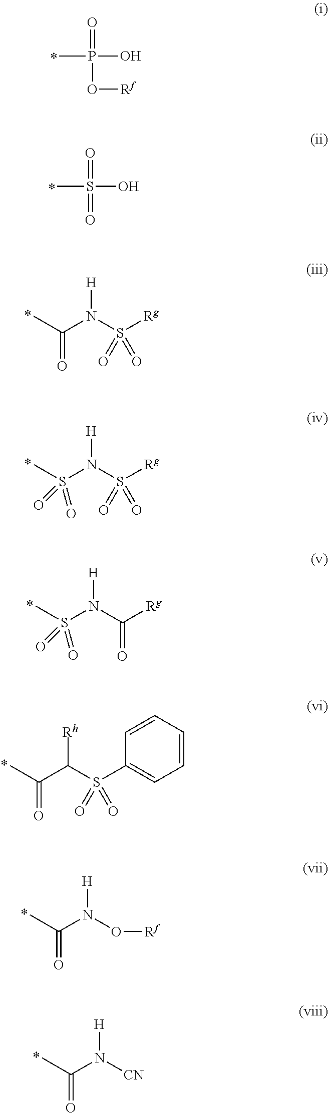

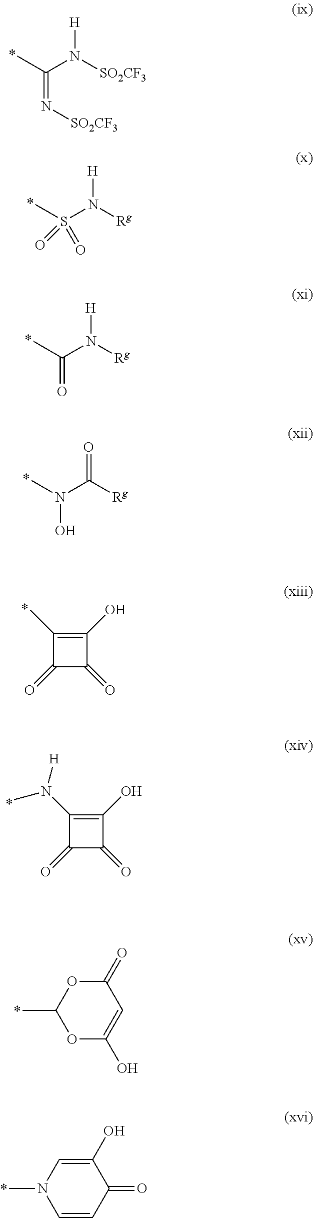

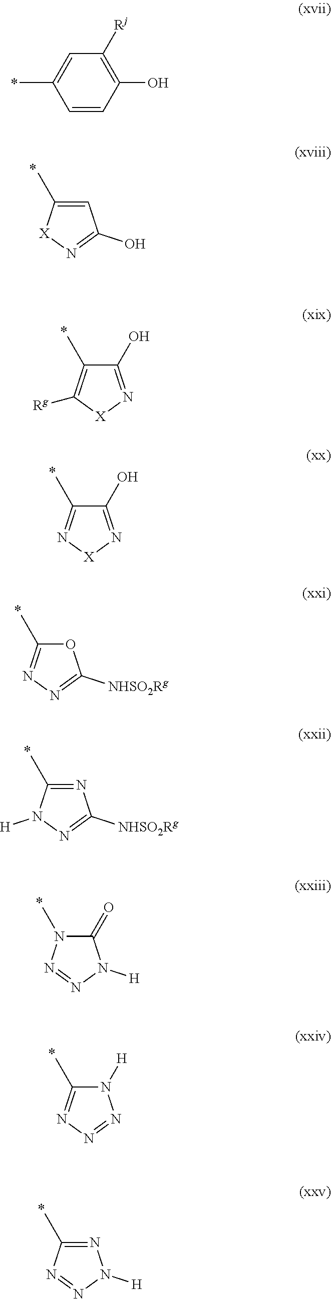

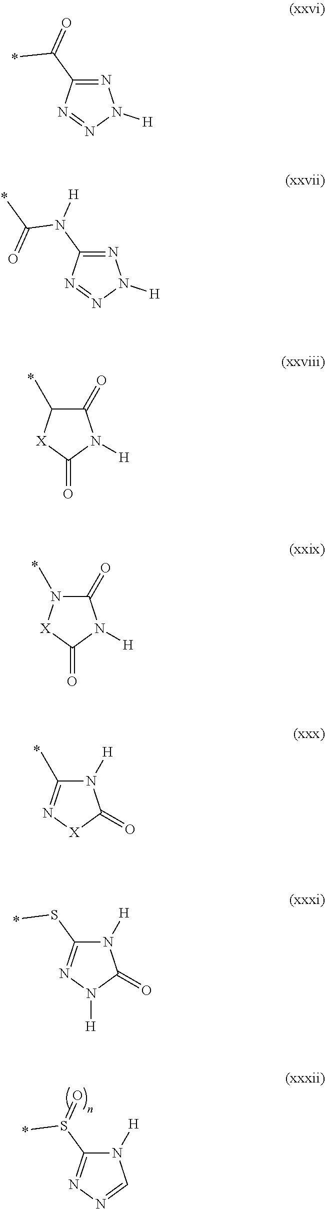

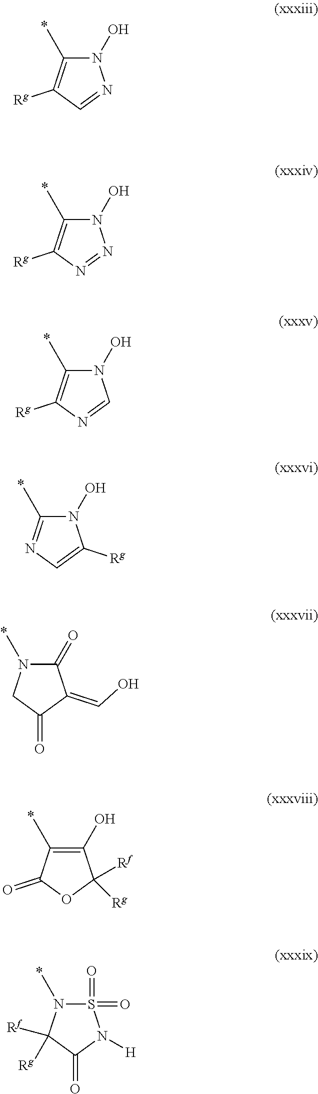

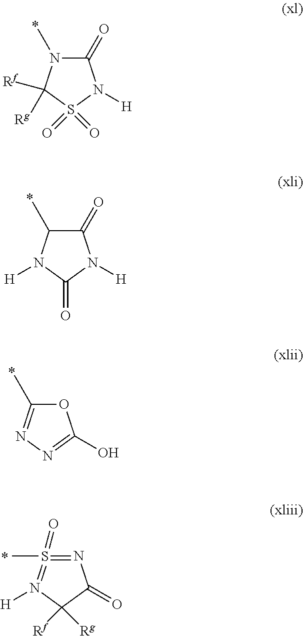

wherein

the asterisk (*) represents the site of attachment to the remainder of the molecule;

n is zero, 1 or 2;

X represents oxygen or sulphur;

R^{f} represents hydrogen, C₁₋₆ alkyl or —CH₂CH(OH)CH₂OH;

R^{g} represents C₁₋₆ alkyl, trifluoromethyl, —CH₂CH₂F, —CH₂CHF₂, —CH₂CF₃ or —CF₂CF₃;

R^{h} represents hydrogen, cyano or —CO₂R^{d}, in which R^{d} is as defined above; and

R^{j} represents hydrogen or halogen.

In one embodiment, n is zero. In another embodiment, n is 1. In a further embodiment, n is 2.

In one embodiment, X represents oxygen. In another embodiment, X represents sulphur.

In one embodiment, R^{f} represents hydrogen. In another embodiment, R^{f} represents C₁₋₆ alkyl, especially methyl. In a further embodiment, R^{f} is —CH₂CH(OH)CH₂OH.

In one embodiment, R^{g} represents C₁₋₆ alkyl, especially methyl. In another embodiment, R^{g} represents trifluoromethyl, —CH₂CH₂F, —CH₂CHF₂, —CH₂CF₃ or —CF₂CF₃.

In a first aspect of that embodiment, R^{g} represents trifluoromethyl. In a second aspect of that embodiment, R^{g} represents —CH₂CH₂F. In a third aspect of that embodiment, R^{g} represents —CH₂CHF₂. In a fourth aspect of that embodiment, R^{g} represents —CH₂CF₃. In a fifth aspect of that embodiment, R^{g} represents —CF₂CF₃.

In one embodiment, R^{h} is hydrogen. In another embodiment, R^{h} represents cyano. In a further embodiment, R^{h} represents —CO₂R^{d}, especially methoxycarbonyl.

In one embodiment, R^{j} represents hydrogen. In another embodiment, R^{j} represents halogen, especially chloro.

In a selected embodiment, Ω represents tetrazolyl, especially a C-linked tetrazolyl moiety of formula (xxiv) or (xxv) as depicted above, in particular a group of formula (xxiv) as depicted above.

In another embodiment, Ω represents C₁₋₆ alkylsulphonylaminocarbonyl, i.e. a moiety of formula (iii) as depicted above wherein R^{g} represents C₁₋₆ alkyl.

In another embodiment, Ω represents C₁₋₆ alkylaminosulphonyl, i.e. a moiety of formula (x) as depicted above wherein R^{g} represents C₁₋₆ alkyl.

In a further embodiment, Ω represents (C₁₋₆)alkylcarbonylaminosulphonyl, i.e. a moiety of formula (v) as depicted above wherein R^{g} represents C₁₋₆ alkyl.

Typical examples of optional substituents which may be present on R¹, R², R³ or R⁴ include one, two or three substituents independently selected from C₁₋₆ alkyl, trifluoromethyl, hydroxy, hydroxy(C₁₋₆)alkyl and (C₁₋₆)alkylsulphoximinyl.

Examples of particular substituents on R¹, R², R³ or R⁴ include fluoro, chloro, bromo, fluoromethyl, fluoroisopropyl, cyano, cyanoethyl, nitro, nitromethyl, methyl, ethyl, isopropyl, isobutyl, tert-butyl, difluoromethyl, trifluoromethyl, difluoroethyl, trifluoro-ethyl, ethenyl, hydroxy, hydroxymethyl, hydroxyisopropyl, methoxy, isopropoxy, difluoromethoxy, trifluoromethoxy, trifluoroethoxy, carboxycyclobutyloxy, methylene-dioxy, ethylenedioxy, methoxymethyl, methoxyethyl, pentafluorothio, methylthio, methylsulphinyl, methylsulphonyl, methylsulphonylethyl, oxo, amino, aminomethyl, aminoisopropyl, methylamino, ethylamino, dimethylamino, hydroxyethylamino, hydroxypropylamino, (hydroxy)(methyl)propylamino, methoxyamino, methoxyethyl-amino, (hydroxy)(methoxy)(methyl)propylamino, (hydroxy)(methylthio)butylamino, N-(hydroxyethyl)-N-(methyl)amino, dimethylaminoethylamino, (dimethylamino)(methyl)-propylamino, N-(dimethylaminoethyl)-N-(hydroxyethyl)amino, hydroxymethyl-cyclopentylamino, hydroxycyclobutylmethylamino, (cyclopropyl)(hydroxy)propylamino, morpholinylethylamino, oxopyrrolidinylmethylamino, ethyloxadiazolylamino, methyl-thiadiazolylamino, thiazolylmethylamino, thiazolylethylamino, pyrimidinylmethylamino, methylpyrazolylmethylamino, acetylamino, N-acetyl-N-methylamino, N-isopropyl-carbonyl-N-methylamino, acetylaminomethyl, ethenylcarbonylamino, bis(ethenyl-carbonyl)amino, N-cyclopropylcarbonyl-N-methylamino, methoxycarbonylamino, ethoxycarbonylamino, tert-butoxycarbonylamino, methoxycarbonylethylamino, ethylaminocarbonylamino, butylaminocarbonylamino, methylsulphonylamino, N-methyl-N-(methylsulphonyl)amino, bis(methylsulphonyl)amino, N-(carboxymethyl)-N-methyl-amino, N-(carboxyethyl)-N-methylamino, carboxycyclopentylamino, carboxycyclopropyl-methylamino, formyl, acetyl, isopropylcarbonyl, cyclobutylcarbonyl, phenylcarbonyl, acetoxyisopropyl, carboxy, carboxymethyl, carboxyethyl, methoxycarbonyl, ethoxycarbonyl, n-butoxycarbonyl, tert-butoxycarbonyl, methoxycarbonylmethyl, ethoxycarbonylmethyl, ethoxycarbonylethyl, morpholinylethoxycarbonyl, ethoxycarbonyl-methylidenyl, methylsulphonylaminocarbonyl, acetylaminosulphonyl, methoxyamino-carbonyl, tetrazolyl, tetrazolylmethyl, hydroxyoxadiazolyl, aminocarbonyl, methylamino-carbonyl, hydroxyethylaminocarbonyl, dimethylaminocarbonyl, aminocarbonylmethyl, aminosulphonyl, methylaminosulphonyl, dimethylaminosulphonyl, methylsulphoximinyl, ethylsulphoximinyl, trifluoromethylsulphoximinyl, (methyl)(N-methyl)sulphoximinyl, (N-carboxymethyl)(methyl)sulphoximinyl, (N-tert-butoxycarbonylmethyl)(methyl)-sulphoximinyl, cyclopropylsulphoximinyl and N-(dimethylsulfoxo)iminyl.

Typical examples of particular substituents on R¹, R², R³ or R⁴ include methyl, ethyl, trifluoromethyl, hydroxy, hydroxyisopropyl and methylsulphoximinyl.

Typically, R¹ represents hydrogen, halogen, cyano or —CO₂R^{d}; or C₁₋₆ alkyl, C₂₋₆ alkynyl, aryl, C₃₋₇ heterocycloalkyl, C₃₋₇ heterocycloalkenyl, heteroaryl, (C₃₋₇)heterocycloalkyl(C₁₋₆)alkyl-aryl-, heteroaryl(C₃₋₇)heterocycloalkyl-, (C₃₋₇)cycloalkyl-heteroaryl-, (C₃₋₇)cycloalkyl(C₁₋₆)alkyl-heteroaryl-, (C₄₋₇)cycloalkenyl-heteroaryl-, (C₄₋₉)bicycloalkyl-heteroaryl-, (C₃₋₇)heterocycloalkyl-heteroaryl-, (C₃₋₇)heterocycloalkyl(C₁₋₆)alkyl-heteroaryl-, (C₃₋₇)heterocycloalkenyl-heteroaryl-, (C₄₋₉)heterobicycloalkyl-heteroaryl- or (C₄₋₉)spiroheterocycloalkyl-heteroaryl-, any of which groups may be optionally substituted by one or more substituents.

Suitably, R¹ represents halogen, cyano or —CO₂R^{d}; or C₁₋₆ alkyl, C₂₋₆ alkynyl, aryl, C₃₋₇ heterocycloalkyl, C₃₋₇ heterocycloalkenyl, heteroaryl, (C₃₋₇)heterocycloalkyl-(C₁₋₆)alkyl-aryl-, heteroaryl(C₃₋₇)heterocycloalkyl-, (C₃₋₇)cycloalkyl-heteroaryl-, (C₃₋₇)cycloalkyl(C₁₋₆)alkyl-heteroaryl-, (C₄₋₇)cycloalkenyl-heteroaryl-, (C₄₋₉)bicycloalkyl-heteroaryl-, (C₃₋₇)heterocycloalkyl-heteroaryl-, (C₃₋₇)heterocycloalkyl(C₁₋₆)alkyl-heteroaryl-, (C₃₋₇)heterocycloalkenyl-heteroaryl-, (C₄₋₉)heterobicycloalkyl-heteroaryl- or (C₄₋₉)spiroheterocycloalkyl-heteroaryl-, any of which groups may be optionally substituted by one or more substituents.

Generally, R¹ represents halogen or cyano; or C₁₋₆ alkyl, C₂₋₆ alkynyl, aryl, C₃₋₇ heterocycloalkyl, C₃₋₇ heterocycloalkenyl, heteroaryl, (C₃₋₇)heterocycloalkyl(C₁₋₆)alkyl-aryl-, heteroaryl(C₃₋₇)heterocycloalkyl-, (C₃₋₇)cycloalkyl-heteroaryl-, (C₃₋₇)cycloalkyl-(C₁₋₆)alkyl-heteroaryl-, (C₄₋₇)cycloalkenyl-heteroaryl-, (C₄₋₉)bicycloalkyl-heteroaryl-, (C₃₋₇)heterocycloalkyl-heteroaryl-, (C₃₋₇)heterocycloalkyl(C₁₋₆)alkyl-heteroaryl-, (C₃₋₇)heterocycloalkenyl-heteroaryl-, (C₄₋₉)heterobicycloalkyl-heteroaryl- or (C₄₋₉)spiroheterocycloalkyl-heteroaryl-, any of which groups may be optionally substituted by one or more substituents.

More generally, R¹ represents halogen; or R¹ represents heteroaryl, (C₃₋₇)cycloalkyl-heteroaryl- or (C₃₋₇)heterocycloalkyl-heteroaryl-, any of which groups may be optionally substituted by one or more substituents.

In a first embodiment, R¹ represents hydrogen.

In a second embodiment, R¹ represents halogen. In one aspect of that embodiment, R¹ represents bromo.

In a third embodiment, R¹ represents cyano.

In a fourth embodiment, R¹ represents —CO₂R^{d}.

In a fifth embodiment, R¹ represents optionally substituted C₁₋₆ alkyl. In one aspect of that embodiment, R¹ represents optionally substituted ethyl.

In a sixth embodiment, R¹ represents optionally substituted C₂₋₆ alkynyl. In one aspect of that embodiment, R¹ represents optionally substituted butynyl.

In a seventh embodiment, R¹ represents optionally substituted aryl. In one aspect of that embodiment, R¹ represents optionally substituted phenyl.

In an eighth embodiment, R¹ represents optionally substituted C₃₋₇ heterocycloalkyl.

In a ninth embodiment, R¹ represents optionally substituted C₃₋₇ heterocycloalkenyl.

In a tenth embodiment, R¹ represents optionally substituted heteroaryl. In selected aspects of that embodiment, R¹ represents benzofuryl, thienyl, indolyl, pyrazolyl, indazolyl, isoxazolyl, thiazolyl, imidazolyl, pyridinyl, quinolinyl, pyridazinyl, pyrimidinyl or pyrazinyl, any of which groups may be optionally substituted by one or more substituents.

In an eleventh embodiment, R¹ represents optionally substituted (C₃₋₇)-heterocycloalkyl(C₁₋₆)alkyl-aryl-. In a first aspect of that embodiment, R¹ represents optionally substituted pyrrolidinylmethylphenyl-. In a second aspect of that embodiment, R¹ represents optionally substituted piperazinylmethylphenyl-.

In a twelfth embodiment, R¹ represents optionally substituted heteroaryl(C₃₋₇)-heterocycloalkyl-. In one aspect of that embodiment, R¹ represents optionally substituted pyridinylpiperazinyl-.

In a thirteenth embodiment, R¹ represents optionally substituted (C₃₋₇)cycloalkyl-heteroaryl-. In a first aspect of that embodiment, R¹ represents optionally substituted cyclohexylpyrazolyl-. In a second aspect of that embodiment, R¹ represents optionally substituted cyclobutylpyridinyl-. In a third aspect of that embodiment, R¹ represents optionally substituted cyclohexylpyridinyl-. In a fourth aspect of that embodiment, R¹ represents optionally substituted cyclopropylpyrimidinyl-. In a fifth aspect of that embodiment, R¹ represents optionally substituted cyclobutylpyrimidinyl-. In a sixth aspect of that embodiment, R¹ represents optionally substituted cyclopentylpyrimidinyl-. In a seventh aspect of that embodiment, R¹ represents optionally substituted cyclohexyl-pyrimidinyl-. In an eighth aspect of that embodiment, R¹ represents optionally substituted cyclohexylpyrazinyl-.

In a fourteenth embodiment, R¹ represents optionally substituted (C₄₋₇)-cycloalkenyl-heteroaryl-.

In a fifteenth embodiment, R¹ represents optionally substituted (C₃₋₇)-heterocycloalkyl-heteroaryl-. In a first aspect of that embodiment, R¹ represents optionally substituted pyrrolidinylpyridinyl-. In a second aspect of that embodiment, R¹ represents optionally substituted tetrahydropyranylpyridinyl-. In a third aspect of that embodiment, R¹ represents optionally substituted piperidinylpyridinyl-. In a fourth aspect of that embodiment, R¹ represents optionally substituted piperazinylpyridinyl-. In a fifth aspect of that embodiment, R¹ represents optionally substituted morpholinylpyridinyl-. In a sixth aspect of that embodiment, R¹ represents optionally substituted thiomorpholinyl-pyridinyl-. In a seventh aspect of that embodiment, R¹ represents optionally substituted diazepanylpyridinyl-. In an eighth aspect of that embodiment, R¹ represents optionally substituted oxetanylpyrimidinyl-. In a ninth aspect of that embodiment, R¹ represents optionally substituted azetidinylpyrimidinyl-. In a tenth aspect of that embodiment, R¹ represents optionally substituted tetrahydrofuranylpyrimidinyl-. In an eleventh aspect of that embodiment, R¹ represents optionally substituted pyrrolidinylpyrimidinyl-. In a twelfth aspect of that embodiment, R¹ represents optionally substituted tetrahydropyranyl-pyrimidinyl-. In a thirteenth aspect of that embodiment, R¹ represents optionally substituted piperidinylpyrimidinyl-. In a fourteenth aspect of that embodiment, R¹ represents optionally substituted piperazinylpyrimidinyl-. In a fifteenth aspect of that embodiment, R¹ represents optionally substituted morpholinylpyrimidinyl-. In a sixteenth aspect of that embodiment, R¹ represents optionally substituted thiomorpholinyl-pyrimidinyl-. In a seventeenth aspect of that embodiment, R¹ represents optionally substituted azepanylpyrimidinyl-. In an eighteenth aspect of that embodiment, R¹ represents optionally substituted oxazepanylpyrimidinyl-. In a nineteenth aspect of that embodiment, R¹ represents optionally substituted diazepanylpyrimidinyl-. In a twentieth aspect of that embodiment, R¹ represents optionally substituted thiadiazepanyl-pyrimidinyl-. In a twenty-first aspect of that embodiment, R¹ represents optionally substituted oxetanylpyrazinyl-. In a twenty-second aspect of that embodiment, R¹ represents optionally substituted piperidinylpyrazinyl-.

In a sixteenth embodiment, R¹ represents optionally substituted (C₃₋₇)-heterocycloalkyl(C₁₋₆)alkyl-heteroaryl-. In a first aspect of that embodiment, R¹ represents optionally substituted morpholinylmethylthienyl-. In a second aspect of that embodiment, R¹ represents optionally substituted morpholinylethylpyrazolyl-.

In a seventeenth embodiment, R¹ represents optionally substituted (C₃₋₇)-heterocycloalkenyl-heteroaryl-.

In an eighteenth embodiment, R¹ represents optionally substituted (C₄₋₉)-heterobicycloalkyl-heteroaryl-.

In a nineteenth embodiment, R¹ represents optionally substituted (C₄₋₉)-spiroheterocycloalkyl-heteroaryl-.

In a twentieth embodiment, R¹ represents optionally substituted (C₃₋₇)cycloalkyl-(C₁₋₆)alkyl-heteroaryl-. In one aspect of that embodiment, R¹ represents optionally substituted cyclohexylmethylpyrimidinyl-.

In a twenty-first embodiment, R¹ represents optionally substituted (C₄₋₉)-bicycloalkyl-heteroaryl-.

Appositely, R¹ represents hydrogen, bromo, iodo or —CO₂R^{d}; or ethyl, butynyl, phenyl, pyrrolidinyl, piperidinyl, piperazinyl, morpholinyl, 1,2,3,6-tetrahydropyridinyl, benzofuryl, thienyl, indolyl, pyrazolyl, indazolyl, isoxazolyl, thiazolyl, imidazolyl, pyridinyl, quinolinyl, pyridazinyl, pyrimidinyl, pyrazinyl, pyrrolidinylmethylphenyl, piperazinylmethylphenyl, pyridinylpiperazinyl, cyclohexylpyrazolyl, cyclobutylpyridinyl, cyclohexylpyridinyl, cyclopropylpyrimidinyl, cyclobutylpyrimidinyl, cyclopentyl-pyrimidinyl, cyclohexylpyrimidinyl, cyclohexylpyrazinyl, cyclohexylmethylpyrimidinyl, cyclohexenylpyridinyl, cyclohexenylpyrimidinyl, bicyclo[3.1.0]hexanylpyridinyl, bicyclo[3.1.0]hexanylpyrimidinyl, bicyclo[4.1.0]heptanylpyrimidinyl, bicyclo[2.2.2]-octanylpyrimidinyl, pyrrolidinylpyridinyl, tetrahydropyranylpyridinyl, piperidinyl-pyridinyl, piperazinylpyridinyl, morpholinylpyridinyl, thiomorpholinylpyridinyl, diazepanylpyridinyl, oxetanylpyrimidinyl, azetidinylpyrimidinyl, tetrahydrofuranyl-pyrimidinyl, pyrrolidinylpyrimidinyl, tetrahydropyranylpyrimidinyl, piperidinyl-pyrimidinyl, piperazinylpyrimidinyl, hexahydro-[1,2,5]thiadiazolo[2,3-a]pyrazinyl-pyrimidinyl, morpholinylpyrimidinyl, thiomorpholinylpyrimidinyl, azepanylpyrimidinyl, oxazepanylpyrimidinyl, diazepanylpyrimidinyl, thiadiazepanylpyrimidinyl, oxetanyl-pyrazinyl, piperidinylpyrazinyl, morpholinylmethylthienyl, morpholinylethylpyrazolyl, 3-azabicyclo[3.1.0]hexanylpyridinyl, 3-azabicyclo[3.1.0]hexanylpyridazinyl, 3-azabicyclo-[3.1.0]hexanylpyrimidinyl, 2-oxa-5-azabicyclo[2.2.1]heptanylpyrimidinyl, 3-azabicyclo-[3.1.1]heptanylpyrimidinyl, 6-oxa-3-azabicyclo[3.1.1]heptanylpyrimidinyl, 3-azabicyclo-[4.1.0]heptanylpyridinyl, 3-azabicyclo[4.1.0]heptanylpyrimidinyl, 2-oxabicyclo[2.2.2]-octanylpyrimidinyl, 3-azabicyclo[3.2.1]octanylpyrimidinyl, 8-azabicyclo[3.2.1]octanyl-pyrimidinyl, 3-oxa-8-azabicyclo[3.2.1]octanylpyrimidinyl, 3,6-diazabicyclo[3.2.2]-nonanylpyrimidinyl, 3-oxa-7-azabicyclo[3.3.1]nonanylpyrimidinyl, 3,7-dioxa-9-azabicyclo[3.3.1]nonanylpyrimidinyl, 5-azaspiro[2.3]hexanylpyrimidinyl, 5-azaspiro-[2.4]heptanylpyrimidinyl, 2-azaspiro[3.3]heptanylpyrimidinyl, 2-oxa-6-azaspiro[3.3]-heptanylpyrimidinyl, 3-oxa-6-azaspiro[3.3]heptanylpyrimidinyl, 6-thia-2-azaspiro[3.3]-heptanylpyrimidinyl, 2-oxa-6-azaspiro[3.4]octanylpyrimidinyl, 2-oxa-6-azaspiro[3.5]-nonanylpyrimidinyl, 2-oxa-7-azaspiro[3.5]nonanylpyrimidinyl or 2,4,8-triazaspiro[4.5]-decanylpyrimidinyl, any of which groups may be optionally substituted by one or more substituents.

Illustratively, R¹ represents bromo; or R¹ represents pyridinyl, pyrimidinyl, cyclobutylpyrimidinyl or azetidinylpyrimidinyl, any of which groups may be optionally substituted by one or more substituents.

Typical examples of optional substituents on R¹ include one, two or three substituents independently selected from halogen, halo(C₁₋₆)alkyl, cyano, cyano(C₁₋₆)alkyl, nitro(C₁₋₆)alkyl, C₁₋₆ alkyl, trifluoromethyl, difluoroethyl, trifluoroethyl, C₂₋₆ alkenyl, hydroxy, hydroxy(C₁₋₆)alkyl, C₁₋₆ alkoxy, trifluoroethoxy, carboxy(C₃₋₇)cycloalkyloxy, pentafluorothio, C₁₋₆ alkylthio, C₁₋₆ alkylsulphonyl, (C₁₋₆)alkylsulphonyl(C₁₋₆)alkyl, oxo, amino, amino(C₁₋₆)alkyl, C₁₋₆ alkylamino, di(C₁₋₆)alkylamino, (C₁₋₆)alkoxy(C₁₋₆)alkyl-amino, N—[(C₁₋₆)alkyl]-N-[hydroxy(C₁₋₆)alkyl]amino, (C₂₋₆)alkylcarbonylamino(C₁₋₆)alkyl, C₁₋₆ alkylsulphonylamino, N—[(C₁₋₆)alkyl]-N—[(C₁₋₆)alkylsulphonyl]amino, bis[(C₁₋₆)alkyl-sulphonyl]amino, N—[(C₁₋₆)alkyl]-N-[carboxy(C₁₋₆)alkyl]amino, carboxy(C₃₋₇)cycloalkyl-amino, carboxy(C₃₋₇)cycloalkyl(C₁₋₆)alkylamino, formyl, C₂₋₆ alkylcarbonyl, (C₂₋₆)alkyl-carbonyloxy(C₁₋₆)alkyl, carboxy, carboxy(C₁₋₆)alkyl, C₂₋₆ alkoxycarbonyl, C₂₋₆ alkoxycarbonyl(C₁₋₆)alkyl, morpholinyl(C₁₋₆)alkoxycarbonyl, C₂₋₆ alkoxycarbonyl-methylidenyl, a carboxylic acid isostere or prodrug moiety Ω as defined herein, —(C₁₋₆)alkyl-Ω, aminocarbonyl, aminosulphonyl, (C₁₋₆)alkylsulphoximinyl, trifluoromethylsulphoximinyl, [(C₁₋₆)alkyl][N—(C₁₋₆)alkyl]sulphoximinyl, [(C₁₋₆)alkyl][N-carboxy(C₁₋₆)alkyl]sulphoximinyl, [N—(C₂₋₆)alkoxycarbonyl(C₁₋₆)alkyl][(C₁₋₆)alkyl]-sulphoximinyl, (C₃₋₇)cycloalkylsulphoximinyl and N-[di(C₁₋₆)alkylsulfoxo]iminyl.

Suitable examples of optional substituents on R¹ include one, two or three substituents independently selected from C₁₋₆ alkyl, trifluoromethyl, hydroxy, hydroxy(C₁₋₆)alkyl and (C₁₋₆)alkylsulphoximinyl.

Typical examples of particular substituents on R¹ include one, two or three substituents independently selected from fluoro, chloro, fluoromethyl, fluoroisopropyl, cyano, cyanoethyl, nitromethyl, methyl, ethyl, isopropyl, trifluoromethyl, difluoroethyl, ethenyl, hydroxy, hydroxymethyl, hydroxyisopropyl, methoxy, isopropoxy, trifluoro-ethoxy, carboxycyclobutyloxy, pentafluorothio, methylthio, methylsulphonyl, methyl-sulphonylethyl, oxo, amino, aminomethyl, aminoisopropyl, methylamino, dimethylamino, methoxyethylamino, N-(hydroxyethyl)-N-(methyl)amino, acetylaminomethyl, methyl-sulphonylamino, N-methyl-N-(methylsulphonyl)amino, bis(methylsulphonyl)amino, N-(carboxyethyl)-N-(methyl)amino, carboxycyclopentylamino, carboxycyclopropylmethyl-amino, formyl, acetyl, acetoxyisopropyl, carboxy, carboxymethyl, carboxyethyl, methoxy-carbonyl, ethoxycarbonyl, n-butoxycarbonyl, tert-butoxycarbonyl, methoxycarbonyl-methyl, ethoxycarbonylmethyl, ethoxycarbonylethyl, morpholinylethoxycarbonyl, ethoxycarbonylmethylidenyl, methylsulphonylaminocarbonyl, acetylaminosulphonyl, methoxyaminocarbonyl, tetrazolyl, tetrazolylmethyl, hydroxyoxadiazolyl, aminocarbonyl, aminosulphonyl, methylsulphoximinyl, ethylsulphoximinyl, trifluoromethylsulphoximinyl, (methyl)(N-methyl)sulphoximinyl, (N-carboxymethyl)(methyl)sulphoximinyl, (N-tert-butoxycarbonylmethyl)(methyl)sulphoximinyl, cyclopropylsulphoximinyl and N-(dimethylsulfoxo)iminyl.

Suitable examples of particular substituents on R¹ include one, two or three substituents independently selected from methyl, ethyl, trifluoromethyl, hydroxy, hydroxyisopropyl and methylsulphoximinyl.

In a particular embodiment, R¹ is substituted by hydroxy(C₁₋₆)alkyl. In one aspect of that embodiment, R¹ is substituted by hydroxyisopropyl, especially 2-hydroxyprop-2-yl.

Selected values of R¹ include hydrogen, bromo, iodo, —CO₂R^{d}, methoxycarbonyl-ethyl, ethoxycarbonylethyl, hydroxybutynyl, chlorophenyl, hydroxyphenyl, pentafluoro-thiophenyl, methylsulphonylphenyl, aminomethylphenyl, aminoisopropylphenyl, acetyl-aminomethylphenyl, acetylphenyl, methoxycarbonylphenyl, aminocarbonylphenyl, aminosulphonylphenyl, acetylaminosulphonylphenyl, methylsulphoximinylphenyl, trifluoromethylsulphoximinylphenyl, (N-carboxymethyl)(methyl)sulphoximinylphenyl, (N-tert-butoxycarbonylmethyl)(methyl)sulphoximinylphenyl, (methoxycarbonyl)-(methyl)pyrrolidinyl, oxopiperidinyl, ethoxycarbonylpiperidinyl, methylsulphonyl-piperazinyl, morpholinyl, methylsulphonyl-1,2,3,6-tetrahydropyridinyl, acetyl-1,2,3,6-tetrahydropyridinyl, tert-butoxycarbonyl-1,2,3,6-tetrahydropyridinyl, methoxycarbonyl-methyl-1,2,3,6-tetrahydropyridinyl, benzofuryl, thienyl, indolyl, pyrazolyl, methyl-pyrazolyl, dimethylpyrazolyl, (methyl)[N-methyl-N-(methylsulfonyl)amino]pyrazolyl, methylindazolyl, dimethylisoxazolyl, hydroxyisopropylthiazolyl, methylimidazolyl, dimethylimidazolyl, pyridinyl, fluoropyridinyl, cyanopyridinyl, methylpyridinyl, (cyano)-(methyl)pyridinyl, dimethylpyridinyl, trifluoromethylpyridinyl, ethenylpyridinyl, hydroxyisopropylpyridinyl, methoxypyridinyl, (methoxy)(methyl)pyridinyl, isopropoxy-pyridinyl, trifluoroethoxypyridinyl, (methyl)(trifluoroethoxy)pyridinyl, methylsulphonyl-pyridinyl, oxopyridinyl, (methyl)(oxo)pyridinyl, (dimethyl)(oxo)pyridinyl, amino-pyridinyl, methylaminopyridinyl, dimethylaminopyridinyl, methoxyethylaminopyridinyl, N-(hydroxyethyl)-N-(methyl)aminopyridinyl, methylsulphonylaminopyridinyl, [bis(methylsulphonyl)amino]pyridinyl, carboxypyridinyl, methylsulphoximinylpyridinyl, ethylsulphoximinylpyridinyl, (methyl)(methylsulphoximinyl)pyridinyl, (methyl)(N-methyl)sulphoximinylpyridinyl, cyclopropylsulphoximinylpyridinyl, N-(dimethyl-sulfoxo)iminylpyridinyl, quinolinyl, hydroxypyridazinyl, pyrimidinyl, fluoroisopropyl-pyrimidinyl, difluoroethylpyrimidinyl, hydroxyisopropylpyrimidinyl, (hydroxyisopropyl)-(methyl)pyrimidinyl, methoxypyrimidinyl, carboxycyclobutyloxypyrimidinyl, methylthiopyrimidinyl, methylsulphonylpyrimidinyl, oxopyrimidinyl, aminopyrimidinyl, dimethylaminopyrimidinyl, methoxyethylaminopyrimidinyl, N-(carboxyethyl)-N-(methyl)aminopyrimidinyl, carboxycyclopentylaminopyrimidinyl, carboxycyclopropyl-methylaminopyrimidinyl, acetoxyisopropylpyrimidinyl, ethoxycarbonylethylpyrimidinyl, hydroxypyrazinyl, hydroxyisopropylpyrazinyl, pyrrolidinylmethylphenyl, piperazinyl-methylphenyl, pyridinylpiperazinyl, carboxycyclohexylpyrazolyl, (dihydroxy)(methyl)-cyclobutylpyridinyl, carboxycyclohexylpyridinyl, fluoromethylcyclopropylpyrimidinyl, hydroxycyclopropylpyrimidinyl, acetylaminomethylcyclopropylpyrimidinyl, hydroxycyclobutylpyrimidinyl, (difluoro)(hydroxy)cyclobutylpyrimidinyl, dihydroxycyclobutylpyrimidinyl, (dihydroxy)(methyl)cyclobutylpyrimidinyl, (dihydroxy)(ethyl)cyclobutylpyrimidinyl, (amino)(hydroxy)cyclobutylpyrimidinyl, (amino)(hydroxy)(methyl)cyclobutylpyrimidinyl, carboxycyclopentylpyrimidinyl, carboxycyclohexylpyrimidinyl, (carboxy)(methyl)cyclohexylpyrimidinyl, (carboxy)-(hydroxy)cyclohexylpyrimidinyl, carboxymethylcyclohexylpyrimidinyl, ethoxycarbonyl-cyclohexylpyrimidinyl, (methoxycarbonyl)(methyl)cyclohexylpyrimidinyl, (ethoxycarbonyl)(methyl)cyclohexylpyrimidinyl, carboxycyclohexylpyrazinyl, carboxycyclohexylmethylpyrimidinyl, carboxycyclohexenylpyridinyl, carboxy-cyclohexenylpyrimidinyl, ethoxycarbonylcyclohexenylpyrimidinyl, carboxybicyclo-[3.1.0]hexanylpyridinyl, carboxybicyclo[3.1.0]hexanylpyrimidinyl, ethoxycarbonyl-bicyclo[3.1.0]hexanylpyrimidinyl, carboxybicyclo[4.1.0]heptanylpyrimidinyl, carboxy-bicyclo[2.2.2]octanylpyrimidinyl, pyrrolidinylpyridinyl, hydroxypyrrolidinylpyridinyl, hydroxytetrahydropyranylpyridinyl, piperidinylpyridinyl, acetylpiperidinylpyridinyl, (carboxy)(methyl)piperidinylpyridinyl, [(carboxy)(methyl)piperidinyl](fluoro)pyridinyl, [(carboxy)(methyl)piperidinyl](chloro)pyridinyl, piperazinylpyridinyl, (methyl)-(piperazinyl)pyridinyl, cyanoethylpiperazinylpyridinyl, trifluoroethylpiperazinylpyridinyl, methylsulphonylpiperazinylpyridinyl, methylsulphonylethylpiperazinylpyridinyl, oxopiperazinylpyridinyl, acetylpiperazinylpyridinyl, (tert-butoxycarbonylpiperazinyl)-(methyl)pyridinyl, carboxymethylpiperazinylpyridinyl, carboxyethylpiperazinylpyridinyl, ethoxycarbonylmethylpiperazinylpyridinyl, ethoxycarbonylethylpiperazinylpyridinyl, morpholinylpyridinyl, thiomorpholinylpyridinyl, oxothiomorpholinylpyridinyl, dioxothiomorpholinylpyridinyl, oxodiazepanylpyridinyl, fluorooxetanylpyrimidinyl, hydroxyoxetanylpyrimidinyl, difluoroazetidinylpyrimidinyl, hydroxyazetidinyl-pyrimidinyl, (hydroxy)(methyl)azetidinylpyrimidinyl, (hydroxy)(trifluoromethyl)-azetidinylpyrimidinyl, carboxyazetidinylpyrimidinyl, (tert-butoxycarbonyl)(hydroxy)-azetidinylpyrimidinyl, tetrazolylazetidinylpyrimidinyl, hydroxytetrahydrofuranyl-pyrimidinyl, hydroxypyrrolidinylpyrimidinyl, carboxypyrrolidinylpyrimidinyl, (carboxy)-(methyl)pyrrolidinylpyrimidinyl, carboxymethylpyrrolidinylpyrimidinyl, ethoxycarbonyl-pyrrolidinylpyrimidinyl, fluorotetrahydropyranylpyrimidinyl, hydroxytetrahydropyranyl-pyrimidinyl, difluoropiperidinylpyrimidinyl, (cyano)(methyl)piperidinylpyrimidinyl, (hydroxy)(nitromethyl)piperidinylpyrimidinyl, (hydroxy)(methyl)piperidinylpyrimidinyl, (hydroxy)(trifluoromethyl)piperidinylpyrimidinyl, (hydroxymethyl)(methyl)piperidinyl-pyrimidinyl, methylsulphonylpiperidinylpyrimidinyl, oxopiperidinylpyrimidinyl, (formyl)(methyl)piperidinylpyrimidinyl, carboxypiperidinylpyrimidinyl, (carboxy)-(fluoro)piperidinylpyrimidinyl, (carboxy)(methyl)piperidinylpyrimidinyl, (carboxy)-(ethyl)piperidinylpyrimidinyl, (carboxy)(trifluoromethyl)piperidinylpyrimidinyl, (carboxy)(hydroxy)piperidinylpyrimidinyl, (carboxy)(hydroxymethyl)piperidinyl-pyrimidinyl, (carboxy)(methoxy)piperidinylpyrimidinyl, (amino)(carboxy)piperidinyl-pyrimidinyl, carboxymethylpiperidinylpyrimidinyl, methoxycarbonylpiperidinyl-pyrimidinyl, ethoxycarbonylpiperidinylpyrimidinyl, (ethoxycarbonyl)(fluoro)piperidinyl-pyrimidinyl, (methoxycarbonyl)(methyl)piperidinylpyrimidinyl, (ethyl)(methoxy-carbonyl)piperidinylpyrimidinyl, (isopropyl)(methoxycarbonyl)piperidinylpyrimidinyl, (ethoxycarbonyl)(methyl)piperidinylpyrimidinyl, (n-butoxycarbonyl)(methyl)piperidinyl-pyrimidinyl, (ethoxycarbonyl)(trifluoromethyl)piperidinylpyrimidinyl, (ethoxycarbonyl)-(hydroxymethyl)piperidinylpyrimidinyl, (methoxy)(methoxycarbonyl)piperidinyl-pyrimidinyl, (carboxy)(methoxycarbonyl)piperidinylpyrimidinyl, (methyl)-(morpholinylethoxycarbonyl)piperidinylpyrimidinyl, ethoxycarbonylmethylpiperidinyl-pyrimidinyl, methylsulphonylaminocarbonylpiperidinylpyrimidinyl, acetylamino-sulphonylpiperidinylpyrimidinyl, methoxyaminocarbonylpiperidinylpyrimidinyl, tetrazolylpiperidinylpyrimidinyl, hydroxyoxadiazolylpiperidinylpyrimidinyl, amino-sulphonylpiperidinylpyrimidinyl, piperazinylpyrimidinyl, methylsulphonylpiperazinyl-pyrimidinyl, oxopiperazinylpyrimidinyl, carboxypiperazinylpyrimidinyl, carboxyethyl-piperazinylpyrimidinyl, tert-butoxycarbonylpiperazinylpyrimidinyl, tetrazolylmethyl-piperazinylpyrimidinyl, trioxohexahydro-[1,2,5]thiadiazolo[2,3-a]pyrazinylpyrimidinyl, morpholinylpyrimidinyl, dimethylmorpholinylpyrimidinyl, hydroxymethylmorpholinyl-pyrimidinyl, carboxymorpholinylpyrimidinyl, (carboxy)(methyl)morpholinylpyrimidinyl, carboxymethylmorpholinylpyrimidinyl, thiomorpholinylpyrimidinyl, dioxo-thiomorpholinylpyrimidinyl, carboxyazepanylpyrimidinyl, carboxyoxazepanyl-pyrimidinyl, oxodiazepanylpyrimidinyl, (oxodiazepanyl)(trifluoromethyl)pyrimidinyl, (oxodiazepanyl)(methoxy)pyrimidinyl, (methyl)(oxo)diazepanylpyrimidinyl, dioxo-thiadiazepanylpyrimidinyl, hydroxyoxetanylpyrazinyl, (carboxy)(methyl)piperidinyl-pyrazinyl, (ethoxycarbonyl)(methyl)piperidinylpyrazinyl, morpholinylmethylthienyl, morpholinylethylpyrazolyl, carboxy-3-azabicyclo[3.1.0]hexanylpyridinyl, carboxy-3-azabicyclo[3.1.0]hexanylpyridazinyl, carboxy-3-azabicyclo[3.1. O]hexanylpyrimidinyl, (carboxy)(methyl)-3-azabicyclo[3.1.0]hexanylpyrimidinyl, methoxycarbonyl-3-azabicyclo[3.1.0]hexanylpyrimidinyl, ethoxycarbonyl-3-azabicyclo[3.1.0]hexanyl-pyrimidinyl, 2-oxa-5-azabicyclo[2.2.1]heptanylpyrimidinyl, carboxy-2-oxa-5-azabicyclo-[2.2.1]heptanylpyrimidinyl, carboxy-3-azabicyclo[3.1.1]heptanylpyrimidinyl, 6-oxa-3-azabicyclo[3.1.1]heptanylpyrimidinyl, carboxy-3-azabicyclo[4.1.0]heptanylpyridinyl, carboxy-3-azabicyclo[4.1.0]heptanylpyrimidinyl, methoxycarbonyl-3-azabicyclo[4.1.0]-heptanylpyrimidinyl, ethoxycarbonyl-3-azabicyclo[4.1.0]heptanylpyrimidinyl, (hydroxy)-(methyl)(oxo)-2-oxabicyclo[2.2.2]octanylpyrimidinyl, carboxy-3-azabicyclo[3.2.1]-octanylpyrimidinyl, methoxycarbonyl-3-azabicyclo[3.2.1]octanylpyrimidinyl, oxo-8-azabicyclo[3.2.1]octanylpyrimidinyl, ethoxycarbonylmethylidenyl-8-azabicyclo[3.2.1]-octanylpyrimidinyl, 3-oxa-8-azabicyclo[3.2.1]octanylpyrimidinyl, oxo-3,6-diazabicyclo-[3.2.2]nonanylpyrimidinyl, carboxy-3-oxa-7-azabicyclo[3.3.1]nonanylpyrimidinyl, 3,7-dioxa-9-azabicyclo[3.3.1]nonanylpyrimidinyl, carboxy-5-azaspiro[2.3]hexanyl-pyrimidinyl, (carboxy)(methyl)-5-azaspiro[2.3]hexanylpyrimidinyl, carboxy-5-azaspiro-[2.4]heptanylpyrimidinyl, carboxy-2-azaspiro[3.3]heptanylpyrimidinyl, 2-oxa-6-azaspiro-[3.3]heptanylpyrimidinyl, 3-oxa-6-azaspiro[3.3]heptanylpyrimidinyl, dioxo-6-thia-2-azaspiro[3.3]heptanylpyrimidinyl, 2-oxa-6-azaspiro[3.4]octanylpyrimidinyl, 2-oxa-6-azaspiro[3.5]nonanylpyrimidinyl, 2-oxa-7-azaspiro[3.5]nonanylpyrimidinyl and (dioxo)(methyl)-2,4,8-triazaspiro[4.5]decanylpyrimidinyl.

Illustrative values of R¹ include bromo, methylsulphoximinylpyridinyl, hydroxyisopropylpyrimidinyl, (dihydroxy)(methyl)cyclobutylpyrimidinyl, (dihydroxy)-(ethyl)cyclobutylpyrimidinyl and (hydroxy)(trifluoromethyl)azetidinylpyrimidinyl.

Typically, R² represents hydrogen, halogen, trifluoromethyl or —OR^{a}; or R² represents optionally substituted C₁₋₆ alkyl.

Suitably, R² represents hydrogen or halogen.

Typical examples of optional substituents on R² include C₂₋₆ alkoxycarbonyl.

Typical examples of particular substituents on R² include ethoxycarbonyl.

In a first embodiment, R² represents hydrogen. In a second embodiment, R² represents halogen. In one aspect of that embodiment, R² represents fluoro. In another aspect of that embodiment, R² represents chloro. In a third embodiment, R² represents trifluoromethyl. In a fourth embodiment, R² represents —OR^{a}. In a fifth embodiment, R² represents optionally substituted C₁₋₆ alkyl. In one aspect of that embodiment, R² represents unsubstituted methyl. In another aspect of that embodiment, R² represents unsubstituted ethyl. In a further aspect of that embodiment, R² represents monosubstituted methyl or monosubstituted ethyl.

Typical values of R² include hydrogen, fluoro, chloro, trifluoromethyl, —OR^{a}, methyl and ethoxycarbonylethyl.

Suitable values of R² include hydrogen and fluoro.

Typically, R³ represents hydrogen, halogen or C₁₋₆ alkyl.

In a first embodiment, R³ represents hydrogen. In a second embodiment, R³ represents halogen. In one aspect of that embodiment, R³ represents fluoro. In a third embodiment, R³ represents C₁₋₆ alkyl. In one aspect of that embodiment, R³ represents methyl. In another aspect of that embodiment, R³ represents ethyl.

In a particular embodiment, R⁴ represents hydrogen.

In a first embodiment, R⁵ represents unsubstituted C₁₋₆ alkyl. In one aspect of that embodiment, R⁵ represents unsubstituted methyl.

In a second embodiment, R⁵ represents C₁₋₆ alkyl substituted by fluoro. In one aspect of that embodiment, R⁵ represents C₂₋₆ alkyl substituted by fluoro, especially 2-fluoroethyl.

In a third embodiment, R⁵ represents C₁₋₆ alkyl substituted by hydroxy. In one aspect of that embodiment, R⁵ represents C₂₋₆ alkyl substituted by hydroxy, especially 2-hydroxyethyl.

In a fourth embodiment, R⁵ represents C₁₋₆ alkyl substituted by C₁₋₆ alkoxy. In one aspect of that embodiment, R⁵ represents C₁₋₆ alkyl substituted by methoxy. In another aspect of that embodiment, R⁵ represents methyl substituted by C₁₋₆ alkoxy. In a particular aspect of that embodiment, R⁵ represents methoxymethyl.

In a fifth embodiment, R⁵ represents C₁₋₆ alkyl substituted by amino. In one aspect of that embodiment, R⁵ represents C₂₋₆ alkyl substituted by amino, especially 2-aminoethyl.

In a sixth embodiment, R⁵ represents C₁₋₆ alkyl substituted by C₁₋₆ alkylamino. In one aspect of that embodiment, R⁵ represents C₁₋₆ alkyl substituted by methylamino. In another aspect of that embodiment, R⁵ represents methyl substituted by C₁₋₆ alkylamino. In a particular aspect of that embodiment, R⁵ represents methylaminomethyl.

In a seventh embodiment, R⁵ represents C₁₋₆ alkyl substituted by di(C₁₋₆)alkyl-amino. In one aspect of that embodiment, R⁵ represents C₁₋₆ alkyl substituted by dimethylamino. In another aspect of that embodiment, R⁵ represents methyl substituted by di(C₁₋₆)alkylamino. In a particular aspect of that embodiment, R⁵ represents dimethyl-aminomethyl.

Appositely, R⁵ represents methyl.

Suitably, R⁶ represents hydrogen or methyl.

In a first embodiment, R⁶ represents hydrogen. In a second embodiment, R⁶ represents C₁₋₆ alkyl, especially methyl.

Suitably, R^{7a} represents hydrogen or methyl.

In a first embodiment, R^{7a} represents hydrogen. In a second embodiment, R^{7a} represents C₁₋₆ alkyl, especially methyl.

Suitably, R^{7b} represents hydrogen or methyl.

In a first embodiment, R^{7b} represents hydrogen. In a second embodiment, R^{7b} represents C₁₋₆ alkyl, especially methyl.

Suitably, R^{8a} represents hydrogen, fluoro or methyl.

In a first embodiment, R^{8a} represents hydrogen. In a second embodiment, R^{8a} represents halogen. In one aspect of that embodiment, R^{8a} represents fluoro. In a third embodiment, R^{8a} represents C₁₋₆ alkyl. In one aspect of that embodiment, R^{8a} represents methyl.

Suitably, R^{8b} represents hydrogen, fluoro or methyl.

In a first embodiment, R^{8b} represents hydrogen. In a second embodiment, R^{8b} represents halogen. In one aspect of that embodiment, R^{8b} represents fluoro. In a third embodiment, R^{b} represents C₁₋₆ alkyl. In one aspect of that embodiment, R^{b} represents methyl.

Alternatively, R^{8a} and R^{8b} may together form an optionally substituted spiro linkage. Thus, R^{8a} and R^{8b}, when taken together with the carbon atom to which they are both attached, may represent C₃₋₇ cycloalkyl or C₃₋₇ heterocycloalkyl, either of which groups may be unsubstituted, or substituted by one or more substituents, typically by one or two substituents. In one embodiment, R^{8a} and R^{8b}, when taken together with the carbon atom to which they are both attached, may suitably represent an optionally substituted cyclopropyl ring. In another embodiment, R^{8a} and R^{8b}, when taken together with the carbon atom to which they are both attached, may suitably represent an optionally substituted oxetanyl ring.

Typical examples of optional substituents on the spirocycle formed by R^{8a} and R^{8b} include C₁₋₆ alkyl, halogen, cyano, trifluoromethyl, hydroxy, C₁₋₆ alkoxy, C₁₋₆ alkylthio, C₁₋₆ alkylsulphinyl, C₁₋₆ alkylsulphonyl, C₂₋₆ alkylcarbonyl, amino, C₁₋₆ alkylamino and di(C₁₋₆)alkylamino.

Typical examples of particular substituents on the spirocycle formed by R^{8a} and R^{8b} include methyl, fluoro, chloro, bromo, cyano, trifluoromethyl, hydroxy, methoxy, methylthio, methylsulphinyl, methylsulphonyl, acetyl, amino, methylamino and dimethylamino.

Alternatively, R^{7a} and R^{8a} may together form an optionally substituted fused bicyclic ring system. Thus, R^{7a} and R^{8a}, when taken together with the two intervening carbon atoms, may represent C₃₋₇ cycloalkyl or C₃₋₇ heterocycloalkyl, either of which groups may be unsubstituted, or substituted by one or more substituents, typically by one or two substituents. In one embodiment, R^{7a} and R^{8a}, when taken together with the two intervening carbon atoms, may suitably represent an optionally substituted cyclopropyl ring. In another embodiment, R^{7a} and R^{8a}, when taken together with the two intervening carbon atoms, may suitably represent an optionally substituted oxetanyl ring.

Typical examples of optional substituents on the fused bicyclic ring system formed by R^{7a} and R^{8a} include C₁₋₆ alkyl, halogen, cyano, trifluoromethyl, hydroxy, C₁₋₆ alkoxy, C₁₋₆ alkylthio, C₁₋₆ alkylsulphinyl, C₁₋₆ alkylsulphonyl, C₂₋₆ alkylcarbonyl, amino, C₁₋₆ alkylamino and di(C₁₋₆)alkylamino.

Typical examples of particular substituents on the fused bicyclic ring system formed by R⁷ and R^{8a} include methyl, fluoro, chloro, bromo, cyano, trifluoromethyl, hydroxy, methoxy, methylthio, methylsulphinyl, methylsulphonyl, acetyl, amino, methylamino and dimethylamino.

Suitably, R⁹ represents hydrogen or methyl.

In a first embodiment, R^{9a} represents hydrogen. In a second embodiment, R^{9a} represents C₁₋₆ alkyl, especially methyl.

Suitably, R^{9b} represents hydrogen or methyl.

In a first embodiment, R^{9b} represents hydrogen. In a second embodiment, R^{9b} represents C₁₋₆ alkyl, especially methyl.

Alternatively, R^{9a} and R^{9b} may together form an optionally substituted spiro linkage. Thus, R^{9a} and R^{9b}, when taken together with the carbon atom to which they are both attached, may represent C₃₋₇ cycloalkyl or C₃₋₇ heterocycloalkyl, either of which groups may be unsubstituted, or substituted by one or more substituents, typically by one or two substituents. In one embodiment, R^{9a} and R^{9b}, when taken together with the carbon atom to which they are both attached, may suitably represent an optionally substituted cyclopropyl ring. In another embodiment, R^{9a} and R^{9b}, when taken together with the carbon atom to which they are both attached, may suitably represent an optionally substituted oxetanyl ring.

Typical examples of optional substituents on the spirocycle formed by R^{9a} and R^{9b} include C₁₋₆ alkyl, halogen, cyano, trifluoromethyl, hydroxy, C₁₋₆ alkoxy, C₁₋₆ alkylthio, C₁₋₆ alkylsulphinyl, C₁₋₆ alkylsulphonyl, C₂₋₆ alkylcarbonyl, amino, C₁₋₆ alkylamino and di(C₁₋₆)alkylamino.

Typical examples of particular substituents on the spirocycle formed by R^{9a} and R^{9b} include methyl, fluoro, chloro, bromo, cyano, trifluoromethyl, hydroxy, methoxy, methylthio, methylsulphinyl, methylsulphonyl, acetyl, amino, methylamino and dimethylamino.

Typical examples of suitable substituents on R^{a}, R^{b}, R^{e}, R^{d} or R^{e}, or on the heterocyclic moiety —NR^{b}R^{c}, include halogen, C₁₋₆ alkyl, C₁₋₆ alkoxy, difluoromethoxy, trifluoromethoxy, C₁₋₆ alkoxy(C₁₋₆)alkyl, C₁₋₆ alkylthio, C₁₋₆ alkylsulphinyl, C₁₋₆ alkylsulphonyl, hydroxy, hydroxy(C₁₋₆)alkyl, amino(C₁₋₆)alkyl, cyano, trifluoromethyl, oxo, C₂₋₆ alkylcarbonyl, carboxy, C₂₋₆ alkoxycarbonyl, C₂₋₆ alkylcarbonyloxy, amino, C₁₋₆ alkylamino, di(C₁₋₆)alkylamino, phenylamino, pyridinylamino, C₂₋₆ alkylcarbonylamino, C₂₋₆ alkylcarbonylamino(C₁₋₆)alkyl, C₂₋₆ alkoxycarbonylamino, C₁₋₆ alkylsulphonylamino, aminocarbonyl, C₁₋₆ alkylaminocarbonyl and di(C₁₋₆)alkylaminocarbonyl.

Typical examples of specific substituents on R^{a}, R^{b}, R^{c}, R^{d} or R^{e}, or on the heterocyclic moiety —NR^{b}R^{c}, include fluoro, chloro, bromo, methyl, ethyl, isopropyl, methoxy, isopropoxy, difluoromethoxy, trifluoromethoxy, methoxymethyl, methylthio, ethylthio, methylsulphinyl, methylsulphonyl, hydroxy, hydroxymethyl, hydroxyethyl, aminomethyl, cyano, trifluoromethyl, oxo, acetyl, carboxy, methoxycarbonyl, ethoxycarbonyl, tert-butoxycarbonyl, acetoxy, amino, methylamino, ethylamino, dimethylamino, phenylamino, pyridinylamino, acetylamino, tert-butoxycarbonylamino, acetylaminomethyl, methylsulphonylamino, aminocarbonyl, methylaminocarbonyl and dimethylaminocarbonyl.

Suitably, R^{a} represents C₁₋₆ alkyl, aryl(C₁₋₆)alkyl or heteroaryl(C₁₋₆)alkyl, any of which groups may be optionally substituted by one or more substituents.

Selected values of R^{a} include methyl, ethyl, benzyl and isoindolylpropyl, any of which groups may be optionally substituted by one or more substituents.

Selected examples of suitable substituents on R^{a} include C₁₋₆ alkoxy and oxo.

Selected examples of specific substituents on R^{a} include methoxy and oxo.

In one embodiment, R^{a} represents optionally substituted C₁₋₆ alkyl. In one aspect of that embodiment, R^{a} ideally represents unsubstituted C₁₋₆ alkyl, especially methyl. In another aspect of that embodiment, R^{a} ideally represents substituted C₁₋₆ alkyl, e.g. methoxyethyl. In another embodiment, R^{a} represents optionally substituted aryl. In one aspect of that embodiment, R^{a} represents unsubstituted aryl, especially phenyl. In another aspect of that embodiment, R^{a} represents monosubstituted aryl, especially methylphenyl. In another embodiment, R^{a} represents optionally substituted aryl(C₁₋₆)alkyl, ideally unsubstituted aryl(C₁₋₆)alkyl, especially benzyl. In a further embodiment, R^{a} represents optionally substituted heteroaryl. In a further embodiment, R^{a} represents optionally substituted heteroaryl(C₁₋₆)alkyl, e.g. dioxoisoindolylpropyl.

Specific values of R^{a} include methyl, methoxyethyl, benzyl and dioxoisoindolyl-propyl.

In a particular aspect, R^{b} represents hydrogen or trifluoromethyl; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents.

Selected values of R^{b} include hydrogen; or C₁₋₆ alkyl, aryl(C₁₋₆)alkyl, C₃₋₇ heterocycloalkyl or C₃₋₇ heterocycloalkyl(C₁₋₆)alkyl, any of which groups may be optionally substituted by one or more substituents.

Typical values of R^{b} include hydrogen and C₁₋₆ alkyl.

Illustratively, R^{b} represents hydrogen or trifluoromethyl; or methyl, ethyl, n-propyl, isopropyl, n-butyl, 2-methylpropyl, tert-butyl, pentyl, hexyl, cyclopropyl, cyclobutyl, cyclopentyl, cyclohexyl, cyclopropylmethyl, cyclobutylmethyl, cyclopentylmethyl, cyclohexylmethyl, phenyl, benzyl, phenylethyl, azetidinyl, tetrahydrofuryl, tetrahydrothienyl, pyrrolidinyl, piperidinyl, homopiperidinyl, morpholinyl, azetidinylmethyl, tetrahydrofurylmethyl, pyrrolidinylmethyl, pyrrolidinylethyl, pyrrolidinylpropyl, thiazolidinylmethyl, imidazolidinylethyl, piperidinylmethyl, piperidinylethyl, tetrahydroquinolinylmethyl, piperazinylpropyl, morpholinylmethyl, morpholinylethyl, morpholinylpropyl, pyridinyl, indolylmethyl, pyrazolylmethyl, pyrazolylethyl, imidazolylmethyl, imidazolylethyl, benzimidazolylmethyl, triazolylmethyl, pyridinylmethyl or pyridinylethyl, any of which groups may be optionally substituted by one or more substituents.

Representative values of R^{b} include hydrogen; or methyl, ethyl, n-propyl, benzyl, pyrrolidinyl or morpholinylpropyl, any of which groups may be optionally substituted by one or more substituents.

Selected examples of suitable substituents on R^{b} include C₁₋₆ alkoxy, C₁₋₆ alkylthio, C₁₋₆ alkylsulphinyl, C₁₋₆ alkylsulphonyl, hydroxy, cyano, C₂₋₆ alkoxycarbonyl, di-(C₁₋₆)alkylamino and C₂₋₆ alkoxycarbonylamino.

Selected examples of specific substituents on R^{b} include methoxy, methylthio, methylsulphinyl, methylsulphonyl, hydroxy, cyano, tert-butoxycarbonyl, dimethylamino and tert-butoxycarbonylamino.

Specific values of R^{b} include hydrogen, methyl, methoxyethyl, methylthioethyl, methylsulphinylethyl, methylsulphonylethyl, hydroxyethyl, cyanoethyl, dimethylamino-ethyl, tert-butoxycarbonylaminoethyl, dihydroxypropyl, benzyl, pyrrolidinyl, tert-butoxycarbonylpyrrolidinyl and morpholinylpropyl.

In one embodiment, R^{b} represents hydrogen. In another embodiment, R^{b} represents C₁₋₆ alkyl, especially methyl.

Selected values of R^{c} include hydrogen; or C₁₋₆ alkyl, C₃₋₇ cycloalkyl or C₃₋₇ heterocycloalkyl, any of which groups may be optionally substituted by one or more substituents.

In a particular aspect, R^{c} represents hydrogen, C₁₋₆ alkyl or C₃₋₇ cycloalkyl.

Representative values of R^{c} include hydrogen; or methyl, cyclobutyl, cyclopentyl, cyclohexyl, tetrahydropyranyl and piperidinyl, any of which groups may be optionally substituted by one or more substituents.

Selected examples of suitable substituents on R^{c} include C₂₋₆ alkylcarbonyl and C₂₋₆ alkoxycarbonyl.

Selected examples of specific substituents on R^{c} include acetyl and tert-butoxycarbonyl.

Specific values of R¹ include hydrogen, methyl, cyclobutyl, cyclopentyl, cyclohexyl, tetrahydropyranyl, acetylpiperidinyl and tert-butoxycarbonylpiperidinyl,

Suitably, R^{c} represents hydrogen or C₁₋₆ alkyl. In one embodiment, R^{c} is hydrogen. In another embodiment, R^{c} represents C₁₋₆ alkyl, especially methyl or ethyl, particularly methyl. In a further embodiment, R^{c} represents C₃₋₇ cycloalkyl, e.g. cyclopropyl, cyclobutyl, cyclopentyl or cyclohexyl.

Alternatively, the moiety —NR^{b}R^{c} may suitably represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents.

Selected examples of suitable substituents on the heterocyclic moiety —NR^{b}R^{c} include C₁₋₆ alkyl, C₁₋₆ alkylsulphonyl, hydroxy, hydroxy(C₁₋₆)alkyl, amino(C₁₋₆)alkyl, cyano, oxo, C₂₋₆ alkylcarbonyl, carboxy, C₂₋₆ alkoxycarbonyl, amino, C₂₋₆ alkylcarbonyl-amino, C₂₋₆ alkylcarbonylamino(C₁₋₆)alkyl, C₂₋₆ alkoxycarbonylamino, C₁₋₆ alkyl-sulphonylamino and aminocarbonyl.

Selected examples of specific substituents on the heterocyclic moiety —NR^{b}R^{c} include methyl, methylsulphonyl, hydroxy, hydroxymethyl, aminomethyl, cyano, oxo, acetyl, carboxy, ethoxycarbonyl, amino, acetylamino, acetylaminomethyl, tert-butoxy-carbonylamino, methylsulphonylamino and aminocarbonyl.

Specific values of the moiety —NR^{b}R^{c} include azetidin-1-yl, hydroxyazetidin-1-yl, hydroxymethylazetidin-1-yl, (hydroxy)(hydroxymethyl)azetidin-1-yl, aminomethyl-azetidin-1-yl, cyanoazetidin-1-yl, carboxyazetidin-1-yl, amino azetidin-1-yl, aminocarbonylazetidin-1-yl, pyrrolidin-1-yl, aminomethylpyrrolidin-1-yl, oxopyrrolidin-1-yl, acetylaminomethylpyrrolidin-1-yl, tert-butoxycarbonylaminopyrrolidin-1-yl, oxo-oxazolidin-3-yl, hydroxyisoxazolidin-2-yl, thiazolidin-3-yl, oxothiazolidin-3-yl, dioxo-isothiazolidin-2-yl, piperidin-1-yl, hydroxypiperidin-1-yl, hydroxymethylpiperidin-1-yl, aminopiperidin-1-yl, acetylaminopiperidin-1-yl, tert-butoxycarbonylaminopiperidin-1-yl, methylsulphonylaminopiperidin-1-yl, morpholin-4-yl, piperazin-1-yl, methylpiperazin-1-yl, methylsulphonylpiperazin-1-yl, oxopiperazin-1-yl, acetylpiperazin-1-yl, ethoxycarbonylpiperazin-1-yl and oxohomopiperazin-1-yl.

Suitably, R^{d} represents hydrogen; or C₁₋₆ alkyl, aryl or heteroaryl, any of which groups may be optionally substituted by one or more substituents.

Selected examples of suitable values for R^{d} include hydrogen, methyl, ethyl, isopropyl, 2-methylpropyl, tert-butyl, cyclopropyl, cyclobutyl, phenyl, thiazolidinyl, thienyl, imidazolyl and thiazolyl, any of which groups may be optionally substituted by one or more substituents.

Selected examples of suitable substituents on R^{d} include halogen, C₁₋₆ alkyl, C₁₋₆ alkoxy, oxo, C₂₋₆ alkylcarbonyloxy and di(C₁₋₆)alkylamino.

Selected examples of particular substituents on R^{d} include fluoro, methyl, methoxy, oxo, acetoxy and dimethylamino.

In one embodiment, R^{d} represents hydrogen. In another embodiment, R^{d} represents optionally substituted C₁₋₆ alkyl. In one aspect of that embodiment, R^{d} ideally represents unsubstituted C₁₋₆ alkyl, e.g. methyl, ethyl, isopropyl, 2-methylpropyl or tert-butyl, especially methyl. In another aspect of that embodiment, R^{d} ideally represents substituted C₁₋₆ alkyl, e.g. substituted methyl or substituted ethyl, including acetoxymethyl, dimethylaminomethyl and trifluoroethyl. In another embodiment, R^{d} represents optionally substituted aryl. In one aspect of that embodiment, R^{d} represents unsubstituted aryl, especially phenyl. In another aspect of that embodiment, R^{d} represents monosubstituted aryl, especially methylphenyl. In a further aspect of that embodiment, R^{d} represents disubstituted aryl, e.g. dimethoxyphenyl. In a further embodiment, R^{d} represents optionally substituted heteroaryl, e.g. thienyl, chlorothienyl, methylthienyl, methylimidazolyl or thiazolyl. In another embodiment, R^{d} represents optionally substituted C₃₋₇ cycloalkyl, e.g. cyclopropyl or cyclobutyl. In a further embodiment, R^{d} represents optionally substituted C₃₋₇ heterocycloalkyl, e.g. thiazolidinyl or oxo-thiazolidinyl.

Selected examples of specific values for R^{d} include hydrogen, methyl, acetoxy-methyl, dimethylaminomethyl, ethyl, trifluoroethyl, isopropyl, 2-methylpropyl, tert-butyl, cyclopropyl, cyclobutyl, phenyl, dimethoxyphenyl, thiazolidinyl, oxothiazolidinyl, thienyl, chlorothienyl, methylthienyl, methylimidazolyl and thiazolyl.

Suitably, R^{e} represents C₁₋₆ alkyl or aryl, either of which groups may be optionally substituted by one or more substituents.

Selected examples of suitable substituents on R^{e} include C₁₋₆ alkyl, especially methyl.

In one embodiment, R^{e} represents optionally substituted C₁₋₆ alkyl, ideally unsubstituted C₁₋₆ alkyl, e.g. methyl or propyl, especially methyl. In another embodiment, R^{e} represents optionally substituted aryl. In one aspect of that embodiment, R^{e} represents unsubstituted aryl, especially phenyl. In another aspect of that embodiment, R^{e} represents monosubstituted aryl, especially methylphenyl. In a further embodiment, R^{e} represents optionally substituted heteroaryl.

Selected values of R^{e} include methyl, propyl and methylphenyl.

One sub-class of compounds according to the invention is represented by the compounds of formula (IIA-1) or (IIA-2) and N-oxides thereof, and pharmaceutically acceptable salts thereof:

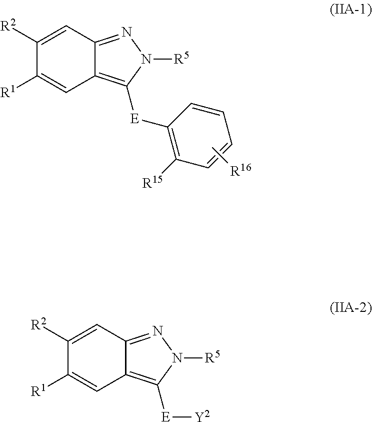

wherein

R¹⁵ and R¹⁶ independently represent hydrogen, halogen, cyano, nitro, C₁₋₆ alkyl, trifluoromethyl, hydroxy, C₁₋₆ alkoxy, difluoromethoxy, trifluoromethoxy, C₁₋₆ alkylthio, C₁₋₆ alkylsulfinyl, C₁₋₆ alkylsulfonyl, amino, C₁₋₆ alkylamino, di(C₁₋₆)alkylamino, arylamino, C₂₋₆ alkylcarbonylamino, C₁₋₆ alkylsulfonylamino, formyl, C₂₋₆ alkylcarbonyl, C₃₋₆ cycloalkylcarbonyl, C₃₋₆ heterocycloalkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl or di(C₁₋₆)alkylaminosulfonyl; and

E, Y², R¹, R² and R⁵ are as defined above.

Typically, R¹⁵ and R¹⁶ may independently represent hydrogen, fluoro, chloro, bromo, cyano, nitro, methyl, isopropyl, trifluoromethyl, hydroxy, methoxy, difluoro-methoxy, trifluoromethoxy, methylthio, methylsulfinyl, methylsulfonyl, amino, methyl-amino, tert-butylamino, dimethylamino, phenylamino, acetylamino, methylsulfonylamino, formyl, acetyl, cyclopropylcarbonyl, azetidinylcarbonyl, pyrrolidinylcarbonyl, piperidinyl-carbonyl, piperazinylcarbonyl, morpholinylcarbonyl, carboxy, methoxycarbonyl, amino-carbonyl, methylaminocarbonyl, dimethylaminocarbonyl, aminosulfonyl, methylamino-sulfonyl and dimethylaminosulfonyl.

Typical values of R¹⁵ include hydrogen, halogen, C₁₋₆ alkyl, trifluoromethyl, C₁₋₆ alkoxy, difluoromethoxy and trifluoromethoxy.

In a first embodiment, R¹⁵ represents hydrogen. In a second embodiment, R¹⁵ represents halogen. In a first aspect of that embodiment, R¹⁵ represents fluoro. In a second aspect of that embodiment, R¹⁵ represents chloro. In a third embodiment, R¹⁵ represents C₁₋₆ alkyl. In one aspect of that embodiment, R¹⁵ represents methyl. In a fourth embodiment, R¹⁵ represents trifluoromethyl. In a fifth embodiment, R¹⁵ represents C₁₋₆ alkoxy. In one aspect of that embodiment, R¹⁵ represents methoxy. In a sixth embodiment, R¹⁵ represents difluoromethoxy. In a seventh embodiment, R¹⁵ represents trifluoromethoxy.

Selected values of R¹⁵ include hydrogen, fluoro, chloro, methyl, trifluoromethyl, methoxy, difluoromethoxy and trifluoromethoxy.

Typical values of R¹⁶ include hydrogen, halogen, cyano, C₁₋₆ alkyl, trifluoro-methyl, difluoromethoxy and amino.

In a first embodiment, R¹⁶ represents hydrogen. In a second embodiment, R¹⁶ represents halogen. In a first aspect of that embodiment, R¹⁶ represents fluoro. In a second aspect of that embodiment, R¹⁶ represents chloro. In a third embodiment, R¹⁶ represents cyano. In a fourth embodiment, R¹⁶ represents C₁₋₆ alkyl. In one aspect of that embodiment, R¹⁶ represents methyl. In a fifth embodiment, R¹⁶ represents trifluoro-methyl. In a sixth embodiment, R¹⁶ represents difluoromethoxy. In a seventh embodiment, R¹⁶ represents amino.

Selected values of R¹⁶ include hydrogen, fluoro, chloro, cyano, methyl, trifluoro-methyl, difluoromethoxy and amino.

In a particular embodiment, R¹⁶ is attached at the para-position of the phenyl ring relative to the integer R¹⁵.

In another embodiment, R¹⁵ and R¹⁶ are attached to the phenyl ring at positions 2 and 6.

A particular sub-group of the compounds of formula (IIA-1) and (IIA-2) above is represented by the compounds of formula (IIB-1) or (IIB-2) and N-oxides thereof, and pharmaceutically acceptable salts thereof:

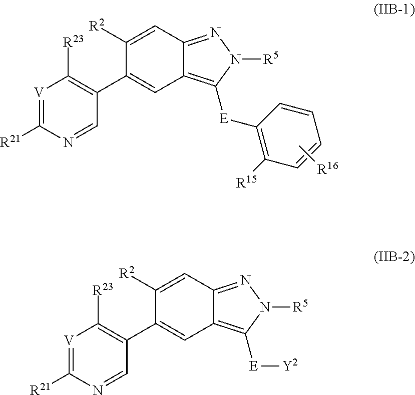

wherein

V represents C—R²² or N;

R²¹ represents hydrogen, halogen, halo(C₁₋₆)alkyl, cyano, C₁₋₆ alkyl, trifluoro-methyl, C₂₋₆ alkenyl, C₂₋₆ alkynyl, hydroxy, hydroxy(C₁₋₆)alkyl, C₁₋₆ alkoxy, (C₁₋₆)alkoxy-(C₁₋₆)alkyl, difluoromethoxy, trifluoromethoxy, trifluoroethoxy, carboxy(C₃₋₇)cycloalkyl-oxy, C₁₋₆ alkylthio, C₁₋₆ alkylsulphonyl, (C₁₋₆)alkylsulphonyl(C₁₋₆)alkyl, amino, amino-(C₁₋₆)alkyl, C₁₋₆ alkylamino, di(C₁₋₆)alkylamino, (C₁₋₆)alkoxy(C₁₋₆)alkylamino, N—[(C₁₋₆)-alkyl]-N-[hydroxy(C₁₋₆)alkyl]amino, C₂₋₆ alkylcarbonylamino, (C₂₋₆)alkylcarbonylamino-(C₁₋₆)alkyl, C₂₋₆ alkoxycarbonylamino, N—[(C₁₋₆)alkyl]-N-[carboxy(C₁₋₆)alkyl]amino, carboxy(C₃₋₇)cycloalkylamino, carboxy(C₃₋₇)cycloalkyl(C₁₋₆)alkylamino, C₁₋₆ alkyl-sulphonylamino, C₁₋₆ alkylsulphonylamino(C₁₋₆)alkyl, formyl, C₂₋₆ alkylcarbonyl, (C₂₋₆)alkylcarbonyloxy(C₁₋₆)alkyl, carboxy, carboxy(C₁₋₆)alkyl, C₂₋₆ alkoxycarbonyl, morpholinyl(C₁₋₆)alkoxycarbonyl, C₂₋₆ alkoxycarbonyl(C₁₋₆)alkyl, C₂₋₆ alkoxycarbonyl-methylidenyl, aminocarbonyl, C₁₋₆ alkylaminocarbonyl, di(C₁₋₆)alkylaminocarbonyl, aminosulphonyl, C₁₋₆ alkylaminosulphonyl, di(C₁₋₆)alkylaminosulphonyl, (C₁₋₆)alkyl-sulphoximinyl or [(C₁₋₆)alkyl][N—(C₁₋₆)alkyl]sulphoximinyl; or R²¹ represents (C₃₋₇)cycloalkyl, (C₃₋₇)cycloalkyl(C₁₋₆)alkyl, (C₄₋₇)cycloalkenyl, (C₄₋₉)bicycloalkyl, (C₃₋₇)heterocycloalkyl, (C₃₋₇)heterocycloalkenyl, (C₄₋₉)heterobicycloalkyl or (C₄₋₉)spiroheterocycloalkyl, any of which groups may be optionally substituted by one or more substituents;

R²² represents hydrogen, halogen or C₁₋₆ alkyl;

R²³ represents hydrogen, C₁₋₆ alkyl, trifluoromethyl or C₁₋₆ alkoxy; and

E, Y², R², R⁵, R¹⁵ and R¹⁶ are as defined above.

In one embodiment, V represents C—R²². In another embodiment, V represents N.

Typically, R²¹ represents hydrogen, halogen, halo(C₁₋₆)alkyl, cyano, C₁₋₆ alkyl, trifluoromethyl, C₂₋₆ alkenyl, hydroxy, hydroxy(C₁₋₆)alkyl, C₁₋₆ alkoxy, trifluoroethoxy, carboxy(C₃₋₇)cycloalkyloxy, C₁₋₆ alkylthio, C₁₋₆ alkylsulphonyl, amino, C₁₋₆ alkylamino, di(C₁₋₆)alkylamino, (C₁₋₆)alkoxy(C₁₋₆)alkylamino, N—[(C₁₋₆)alkyl]-N-[hydroxy(C₁₋₆)alkyl]-amino, N—[(C₁₋₆)alkyl]-N-[carboxy(C₁₋₆)alkyl]amino, carboxy(C₃₋₇)cycloalkylamino, carboxy(C₃₋₇)cycloalkyl(C₁₋₆)alkylamino, C₁₋₆ alkylsulphonylamino, (C₂₋₆)alkylcarbonyl-oxy(C₁₋₆)alkyl, carboxy, morpholinyl(C₁₋₆)alkoxycarbonyl, C₂₋₆ alkoxycarbonyl(C₁₋₆)alkyl, C₂₋₆ alkoxycarbonylmethylidenyl, (C₁₋₆)alkylsulphoximinyl or [(C₁₋₆)alkyl][N—(C₁₋₆)alkyl]-sulphoximinyl; or R²¹ represents (C₃₋₇)cycloalkyl, (C₃₋₇)cycloalkyl(C₁₋₆)alkyl, (C₄₋₇)cycloalkenyl, (C₄₋₉)bicycloalkyl, (C₃₋₇)heterocycloalkyl, (C₄₋₉)heterobicycloalkyl or (C₄₋₉)spiroheterocycloalkyl, any of which groups may be optionally substituted by one or more substituents.

Suitably, R²¹ represents hydroxy(C₁₋₆)alkyl; or R²¹ represents (C₃₋₇)heterocycloalkyl, which group may be optionally substituted by one or more substituents.

Where R²¹ represents an optionally substituted (C₃₋₇)cycloalkyl group, typical values include cyclopropyl, cyclobutyl, cyclopentyl, cyclohexyl and cycloheptyl, any of which groups may be optionally substituted by one or more substituents.

Where R²¹ represents an optionally substituted (C₃₋₇)cycloalkyl(C₁₋₆)alkyl group, a typical value is cyclohexylmethyl, which group may be optionally substituted by one or more substituents.

Where R²¹ represents an optionally substituted (C₄₋₇)cycloalkenyl group, typical values include cyclobutenyl, cyclopentenyl, cyclohexenyl and cycloheptenyl, any of which groups may be optionally substituted by one or more substituents.

Where R²¹ represents an optionally substituted (C₄₋₉)bicycloalkyl group, typical values include bicyclo[3.1.0]hexanyl, bicyclo[4.1.0]heptanyl and bicyclo[2.2.2]octanyl, any of which groups may be optionally substituted by one or more substituents.

Where R²¹ represents an optionally substituted (C₃₋₇)heterocycloalkyl group, typical values include oxetanyl, azetidinyl, tetrahydrofuranyl, pyrrolidinyl, tetrahydro-pyranyl, piperidinyl, piperazinyl, hexahydro-[1,2,5]thiadiazolo[2,3-a]pyrazinyl, morpholinyl, thiomorpholinyl, azepanyl, oxazepanyl, diazepanyl and thiadiazepanyl, any of which groups may be optionally substituted by one or more substituents.

Where R²¹ represents an optionally substituted (C₃₋₇)heterocycloalkenyl group, a typical value is optionally substituted 1,2,3,6-tetrahydropyridinyl.

Where R²¹ represents an optionally substituted (C₄₋₉)heterobicycloalkyl group, typical values include 3-azabicyclo[3.1.0]hexanyl, 2-oxa-5-azabicyclo[2.2.1]heptanyl, 3-azabicyclo[3.1.1]heptanyl, 6-oxa-3-azabicyclo[3.1.1]heptanyl, 3-azabicyclo[4.1.0]-heptanyl, 2-oxabicyclo[2.2.2]octanyl, quinuclidinyl, 2-oxa-5-azabicyclo[2.2.2]octanyl, 3-azabicyclo[3.2.1]octanyl, 8-azabicyclo[3.2.1]octanyl, 3-oxa-8-azabicyclo[3.2.1]octanyl, 3,8-diazabicyclo[3.2.1]octanyl, 3,6-diazabicyclo[3.2.2]nonanyl, 3-oxa-7-azabicyclo-[3.3.1]nonanyl, 3,7-dioxa-9-azabicyclo[3.3.1]nonanyl and 3,9-diazabicyclo[4.2.1]nonanyl, any of which groups may be optionally substituted by one or more substituents.

Where R²¹ represents an optionally substituted (C₄₋₉)spiroheterocycloalkyl group, typical values include 5-azaspiro[2.3]hexanyl, 5-azaspiro[2.4]heptanyl, 2-azaspiro[3.3]-heptanyl, 2-oxa-6-azaspiro[3.3]heptanyl, 3-oxa-6-azaspiro[3.3]heptanyl, 6-thia-2-azaspiro[3.3]heptanyl, 2-oxa-6-azaspiro[3.4]octanyl, 2-oxa-6-azaspiro[3.5]nonanyl, 2-oxa-7-azaspiro[3.5]nonanyl and 2,4,8-triazaspiro[4.5]decanyl, any of which groups may be optionally substituted by one or more substituents.

Illustratively, R²¹ represents hydroxy, hydroxy(C₁₋₆)alkyl, methoxy, carboxy-cyclobutyloxy, methylthio, methylsulphonyl, methylamino, N-[carboxyethyl]-N-methyl-amino, carboxycyclopentylamino, carboxycyclopropylmethylamino, ethoxycarbonylethyl, methylsulphoximinyl, ethylsulphoximinyl or (methyl)(N-methyl)sulphoximinyl; or R²¹ represents cyclopropyl, cyclobutyl, cyclopentyl, cyclohexyl, cyclohexylmethyl, cyclohexenyl, bicyclo[3.1.0]hexanyl, bicyclo[4.1.0]heptanyl, bicyclo[2.2.2]octanyl, oxetanyl, azetidinyl, tetrahydrofuranyl, pyrrolidinyl, tetrahydropyranyl, piperidinyl, piperazinyl, hexahydro-[1,2,5]thiadiazolo[2,3-a]pyrazinyl, morpholinyl, thiomorpholinyl, azepanyl, oxazepanyl, diazepanyl, thiadiazepanyl, 3-azabicyclo[3.1.0]hexanyl, 2-oxa-5-azabicyclo[2.2.1]heptanyl, 3-azabicyclo[3.1.1]heptanyl, 6-oxa-3-azabicyclo[3.1.1]-heptanyl, 3-azabicyclo[4.1.0]heptanyl, 2-oxabicyclo[2.2.2]octanyl, 3-azabicyclo[3.2.1]-octanyl, 8-azabicyclo[3.2.1]octanyl, 3-oxa-8-azabicyclo[3.2.1]octanyl, 3,6-diazabicyclo-[3.2.2]nonanyl, 3-oxa-7-azabicyclo[3.3.1]nonanyl, 3,7-dioxa-9-azabicyclo[3.3.1]nonanyl, 5-azaspiro[2.3]hexanyl, 5-azaspiro[2.4]heptanyl, 2-azaspiro[3.3]heptanyl, 3-oxa-6-azaspiro[3.3]heptanyl or 6-thia-2-azaspiro[3.3]heptanyl, any of which groups may be optionally substituted by one or more substituents.

Appositely, R²¹ represents hydroxy(C₁₋₆)alkyl; or R²¹ represents azetidinyl, which group may be optionally substituted by one or more substituents.

Examples of optional substituents which may be present on R²¹ include one, two or three substituents independently selected from halogen, halo(C₁₋₆)alkyl, cyano, cyano-(C₁₋₆)alkyl, nitro, nitro(C₁₋₆)alkyl, C₁₋₆ alkyl, trifluoromethyl, trifluoroethyl, C₂₋₆ alkenyl, hydroxy, hydroxy(C₁₋₆)alkyl, C₁₋₆ alkoxy, difluoromethoxy, trifluoromethoxy, trifluoro-ethoxy, C₁₋₆ alkylthio, C₁₋₆ alkylsulphonyl, (C₁₋₆)alkylsulphonyl(C₁₋₆)alkyl, oxo, amino, C₁₋₆ alkylamino, di(C₁₋₆)alkylamino, C₂₋₆ alkylcarbonylamino, (C₂₋₆)alkylcarbonylamino-(C₁₋₆)alkyl, C₂₋₆ alkoxycarbonylamino, C₁₋₆ alkylsulphonylamino, formyl, C₂₋₆ alkylcarbonyl, carboxy, carboxy(C₁₋₆)alkyl, C₂₋₆ alkoxycarbonyl, morpholinyl-(C₁₋₆)alkoxycarbonyl, C₂₋₆ alkoxycarbonyl(C₁₋₆)alkyl, C₂₋₆ alkoxycarbonylmethylidenyl, a carboxylic acid isostere or prodrug moiety Ω as defined herein, —(C₁₋₆)alkyl-Ω, amino-carbonyl, C₁₋₆ alkylaminocarbonyl, di(C₁₋₆)alkylaminocarbonyl, aminosulphonyl, di(C₁₋₆)alkylaminosulphonyl, (C₁₋₆)alkylsulphoximinyl and [(C₁₋₆)alkyl][N—(C₁₋₆)alkyl]-sulphoximinyl.

Typical examples of optional substituents on R²¹ include one, two or three substituents independently selected from trifluoromethyl and hydroxy.

Suitable examples of particular substituents on R²¹ include one, two or three substituents independently selected from fluoro, fluoromethyl, chloro, bromo, cyano, cyanomethyl, cyanoethyl, nitro, nitromethyl, methyl, ethyl, isopropyl, trifluoromethyl, trifluoroethyl, ethenyl, hydroxy, hydroxymethyl, methoxy, ethoxy, difluoromethoxy, trifluoromethoxy, trifluoroethoxy, methylthio, methylsulphonyl, methylsulphonylmethyl, methylsulphonylethyl, oxo, amino, methylamino, dimethylamino, acetylamino, acetyl-aminomethyl, methoxycarbonylamino, ethoxycarbonylamino, tert-butoxycarbonylamino, methylsulphonylamino, formyl, acetyl, carboxy, carboxymethyl, carboxyethyl, methoxycarbonyl, ethoxycarbonyl, n-butoxycarbonyl, tert-butoxycarbonyl, morpholinyl-ethoxycarbonyl, methoxycarbonylmethyl, ethoxycarbonylmethyl, ethoxycarbonylethyl, ethoxycarbonylmethylidenyl, acetylaminosulphonyl, methoxyaminocarbonyl, tetrazolyl, tetrazolylmethyl, hydroxyoxadiazolyl, aminocarbonyl, methylaminocarbonyl, dimethyl-aminocarbonyl, methylsulphonylaminocarbonyl, aminosulphonyl, methylaminosulphonyl, dimethylaminosulphonyl, methylsulphoximinyl and (methyl)(N-methyl)sulphoximinyl.

Typical examples of particular substituents on R²¹ include one, two or three substituents independently selected from trifluoromethyl and hydroxy.

Typically, R²¹ represents hydrogen, fluoro, fluoroisopropyl, cyano, methyl, trifluoromethyl, ethenyl, hydroxy, hydroxyisopropyl, methoxy, isopropoxy, trifluoro-ethoxy, carboxycyclobutyloxy, methylthio, methylsulphonyl, amino, methylamino, dimethylamino, methoxyethylamino, N-(hydroxyethyl)-N-(methyl)amino, N-[carboxy-ethyl]-N-methylamino, carboxycyclopentylamino, carboxycyclopropylmethylamino, methylsulphonylamino, acetoxyisopropyl, carboxy, ethoxycarbonylethyl, methyl-sulphoximinyl, ethylsulphoximinyl, (methyl)(N-methyl)sulphoximinyl, fluoromethyl-cyclopropyl, hydroxycyclopropyl, (difluoro)(hydroxy)cyclopropyl, acetylaminomethyl-cyclopropyl, hydroxycyclobutyl, (difluoro)(hydroxy)cyclobutyl, (dihydroxy)cyclobutyl, (dihydroxy)(methyl)cyclobutyl, (dihydroxy)(ethyl)cyclobutyl, (amino)(hydroxy)-cyclobutyl, (amino)(hydroxy)(methyl)cyclobutyl, carboxycyclopentyl, carboxycyclohexyl, (carboxy)(methyl)cyclohexyl, (carboxy)(hydroxy)cyclohexyl, carboxymethylcyclohexyl, ethoxycarbonylcyclohexyl, (methoxycarbonyl)(methyl)cyclohexyl, (ethoxycarbonyl)-(methyl)cyclohexyl, carboxycyclohexylmethyl, carboxycyclohexenyl, ethoxycarbonyl-cyclohexenyl, carboxybicyclo[3.1.0]hexanyl, ethoxycarbonylbicyclo[3.1.0]hexanyl, carboxybicyclo[4.1.0]heptanyl, carboxybicyclo[2.2.2]octanyl, fluorooxetanyl, hydroxyoxetanyl, difluoroazetidinyl, hydroxyazetidinyl, (hydroxy)(methyl)azetidinyl, (hydroxy)(trifluoromethyl)azetidinyl, carboxyazetidinyl, (tert-butoxycarbonyl)(hydroxy)-azetidinyl, tetrazolylazetidinyl, hydroxytetrahydrofuranyl, pyrrolidinyl, hydroxy-pyrrolidinyl, carboxypyrrolidinyl, (carboxy)(methyl)pyrrolidinyl, carboxymethyl-pyrrolidinyl, ethoxycarbonylpyrrolidinyl, fluorotetrahydropyranyl, hydroxytetrahydro-pyranyl, piperidinyl, difluoropiperidinyl, (cyano)(methyl)piperidinyl, (hydroxy)-(nitromethyl)piperidinyl, (hydroxy)(methyl)piperidinyl, (hydroxy)(trifluoromethyl)-piperidinyl, (hydroxymethyl)(methyl)piperidinyl, methylsulphonylpiperidinyl, oxopiperidinyl, (formyl)(methyl)piperidinyl, acetylpiperidinyl, carboxypiperidinyl, (carboxy)(fluoro)piperidinyl, (carboxy)(methyl)piperidinyl, (carboxy)(ethyl)piperidinyl, (carboxy)(trifluoromethyl)piperidinyl, (carboxy)(hydroxy)piperidinyl, (carboxy)-(hydroxymethyl)piperidinyl, (carboxy)(methoxy)piperidinyl, (amino)(carboxy)piperidinyl, carboxymethylpiperidinyl, methoxycarbonylpiperidinyl, (methoxycarbonyl)(methyl)-piperidinyl, (ethyl)(methoxycarbonyl)piperidinyl, (isopropyl)(methoxycarbonyl)-piperidinyl, (methoxy)(methoxycarbonyl)piperidinyl, (carboxy)(methoxycarbonyl)-piperidinyl, ethoxycarbonylpiperidinyl, (ethoxycarbonyl)(fluoro)piperidinyl, (ethoxycarbonyl)(methyl)piperidinyl, (ethoxycarbonyl)(trifluoromethyl)piperidinyl, (ethoxycarbonyl)(hydroxymethyl)piperidinyl, (n-butoxycarbonyl)(methyl)piperidinyl, (methyl)(morpholinylethoxycarbonyl)piperidinyl, ethoxycarbonylmethylpiperidinyl, methylsulphonylaminocarbonylpiperidinyl, acetylaminosulphonylpiperidinyl, methoxyaminocarbonylpiperidinyl, tetrazolylpiperidinyl, hydroxyoxadiazolylpiperidinyl, aminosulphonylpiperidinyl, piperazinyl, cyanoethylpiperazinyl, trifluoroethylpiperazinyl, methylsulphonylpiperazinyl, methylsulphonylethylpiperazinyl, oxopiperazinyl, acetyl-piperazinyl, carboxypiperazinyl, tert-butoxycarbonylpiperazinyl, carboxymethyl-piperazinyl, carboxyethylpiperazinyl, ethoxycarbonylmethylpiperazinyl, ethoxycarbonyl-ethylpiperazinyl, tetrazolylmethylpiperazinyl, trioxohexahydro-[1,2,5]thiadiazolo[2,3-a]-pyrazinyl, morpholinyl, dimethylmorpholinyl, hydroxymethylmorpholinyl, carboxy-morpholinyl, (carboxy)(methyl)morpholinyl, carboxymethylmorpholinyl, thiomorpholinyl, oxothiomorpholinyl, dioxothiomorpholinyl, carboxyazepanyl, carboxyoxazepanyl, oxodiazepanyl, (methyl)(oxo)diazepanyl, dioxothiadiazepanyl, carboxy-3-azabicyclo-[3.1.0]hexanyl, (carboxy)(methyl)-3-azabicyclo[3.1.0]hexanyl, methoxycarbonyl-3-azabicyclo[3.1.0]hexanyl, ethoxycarbonyl-3-azabicyclo[3.1.0]hexanyl, 2-oxa-5-azabicyclo[2.2.1]heptanyl, carboxy-2-oxa-5-azabicyclo[2.2.1]heptanyl, carboxy-3-azabicyclo[3.1.1]heptanyl, 6-oxa-3-azabicyclo[3.1.1]heptanyl, carboxy-3-azabicyclo-[4.1.0]heptanyl, methoxycarbonyl-3-azabicyclo[4.1.0]heptanyl, ethoxycarbonyl-3-azabicyclo[4.1.0]heptanyl, (hydroxy)(methyl)(oxo)-2-oxabicyclo[2.2.2]octanyl, carboxy-3-azabicyclo[3.2.1]octanyl, methoxycarbonyl-3-azabicyclo[3.2.1]octanyl, oxo-8-azabicyclo[3.2.1]octanyl, ethoxycarbonylmethylidenyl-8-azabicyclo[3.2.1]octanyl, 3-oxa-8-azabicyclo[3.2.1]octanyl, oxo-3,6-diazabicyclo[3.2.2]nonanyl, carboxy-3-oxa-7-azabicyclo[3.3.1]nonanyl, 3,7-dioxa-9-azabicyclo[3.3.1]nonanyl, carboxy-5-azaspiro-[2.3]hexanyl, (carboxy)(methyl)-5-azaspiro[2.3]hexanyl, carboxy-5-azaspiro[2.4]heptanyl, carboxy-2-azaspiro[3.3]heptanyl, 2-oxa-6-azaspiro[3.3]heptanyl, 3-oxa-6-azaspiro[3.3]-heptanyl, dioxo-6-thia-2-azaspiro[3.3]heptanyl, 2-oxa-6-azaspiro[3.4]octanyl, 2-oxa-6-azaspiro[3.5]nonanyl, 2-oxa-7-azaspiro[3.5]nonanyl or (dioxo)(methyl)-2,4,8-triazaspiro-[4.5]decanyl.

Illustrative values of R²¹ include hydroxyisopropyl and (hydroxy)(trifluoromethyl)-azetidinyl.

In a particular embodiment, R²¹ represents hydroxy(C₁₋₆)alkyl. In one aspect of that embodiment, R²¹ represents hydroxyisopropyl, especially 2-hydroxyprop-2-yl.

Generally, R²² represents hydrogen or C₁₋₆ alkyl.

Suitably, R²² represents hydrogen, chloro or methyl.

Typically, R²² represents hydrogen or methyl.

In one embodiment, R²² represents hydrogen. In another embodiment, R²² represents C₁₋₆ alkyl, especially methyl. In a further embodiment, R²² represents halogen. In one aspect of that embodiment, R²² represents fluoro. In another aspect of that embodiment, R²² represents chloro.

Generally, R²³ represents hydrogen or C₁₋₆ alkyl.

Suitably, R²³ represents hydrogen, methyl, trifluoromethyl or methoxy.

Typically, R²³ represents hydrogen or methyl.

In one embodiment, R²³ represents hydrogen. In another embodiment, R²³ represents C₁₋₆ alkyl, especially methyl. In a further embodiment, R²³ represents trifluoromethyl. In an additional embodiment, R²³ represents C₁₋₆ alkoxy, especially methoxy.

Particular sub-groups of the compounds of formula (IIB-1) above are represented by the compounds of formula (IIC-1) and (IID-1) and N-oxides thereof, and pharmaceutically acceptable salts thereof:

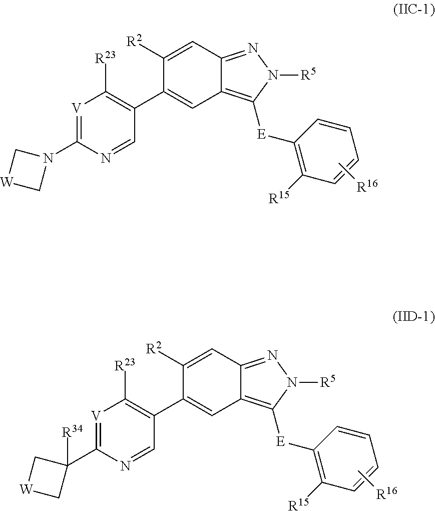

wherein

W represents O, S, S(O), S(O)₂, S(O)(NR⁶), N(R³¹) or C(R³²)(R³³);

R³¹ represents hydrogen, cyano(C₁₋₆)alkyl, C₁₋₆ alkyl, trifluoromethyl, trifluoro-ethyl, C₁₋₆ alkylsulphonyl, (C₁₋₆)alkylsulphonyl(C₁₋₆)alkyl, formyl, C₂₋₆ alkylcarbonyl, carboxy, carboxy(C₁₋₆)alkyl, C₂₋₆ alkoxycarbonyl, C₂₋₆ alkoxycarbonyl(C₁₋₆)alkyl, a carboxylic acid isostere or prodrug moiety Ω, —(C₁₋₆)alkyl-Ω, aminocarbonyl, C₁₋₆ alkylaminocarbonyl, di(C₁₋₆)alkylaminocarbonyl, aminosulphonyl or di(C₁₋₆)alkylamino-sulphonyl;

R³² represents hydrogen, halogen, cyano, hydroxy, hydroxy(C₁₋₆)alkyl, C₁₋₆ alkylsulphonyl, formyl, C₂₋₆ alkylcarbonyl, carboxy, carboxy(C₁₋₆)alkyl, C₂₋₆ alkoxycarbonyl, C₂₋₆ alkoxycarbonyl(C₁₋₆)alkyl, aminosulphonyl, (C₁₋₆)alkyl-sulphoximinyl, [(C₁₋₆)alkyl][N—(C₁₋₆)alkyl]sulphoximinyl, a carboxylic acid isostere or prodrug moiety Ω, or —(C₁₋₆)alkyl-Ω;

R³³ represents hydrogen, halogen, C₁₋₆ alkyl, trifluoromethyl, hydroxy, hydroxy-(C₁₋₆)alkyl, C₁₋₆ alkoxy, amino or carboxy;

R³⁴ represents hydrogen, halogen, halo(C₁₋₆)alkyl, hydroxy, C₁₋₆ alkoxy, C₁₋₆ alkylthio, C₁₋₆ alkylsulphinyl, C₁₋₆ alkylsulphonyl, amino, C₁₋₆ alkylamino, di(C₁₋₆)alkyl-amino, (C₂₋₆)alkylcarbonylamino, (C₂₋₆)alkylcarbonylamino(C₁₋₆)alkyl, (C₁₋₆)alkyl-sulphonylamino or (C₁₋₆)alkylsulphonylamino(C₁₋₆)alkyl; and

V, E, R², R⁵, R⁶, R¹⁵, R¹⁶, R²³ and Ω are as defined above.

Generally, W represents O, S(O)₂, N(R³¹) or C(R³²)(R³³).

Typically, W represents O, N(R³¹) or C(R³²)(R³³).

In a first embodiment, W represents O. In a second embodiment, W represents S. In a third embodiment, W represents S(O). In a fourth embodiment, W represents S(O)₂. In a fifth embodiment, W represents S(O)(NR⁶). In a sixth embodiment, W represents N(R³¹). In a seventh embodiment, W represents C(R³²)(R³³).

Typically, R³¹ represents hydrogen, cyano(C₁₋₆)alkyl, C₁₋₆ alkyl, trifluoromethyl, trifluoroethyl, C₁₋₆ alkylsulphonyl, (C₁₋₆)alkylsulphonyl(C₁₋₆)alkyl, formyl, C₂₋₆ alkylcarbonyl, carboxy, carboxy(C₁₋₆)alkyl, C₂₋₆ alkoxycarbonyl, C₂₋₆ alkoxycarbonyl-(C₁₋₆)alkyl, tetrazolyl(C₁₋₆)alkyl, aminocarbonyl, C₁₋₆ alkylaminocarbonyl, di(C₁₋₆)alkyl-aminocarbonyl, aminosulphonyl, C₁₋₆ alkylaminosulphonyl or di(C₁₋₆)alkylamino-sulphonyl.

Typical values of R³¹ include hydrogen, cyanoethyl, methyl, ethyl, isopropyl, trifluoromethyl, trifluoroethyl, methylsulphonyl, methylsulphonylethyl, formyl, acetyl, carboxy, carboxymethyl, carboxyethyl, methoxycarbonyl, ethoxycarbonyl, tert-butoxy-carbonyl, ethoxycarbonylmethyl, ethoxycarbonylethyl, tetrazolylmethyl, aminocarbonyl, methylamino-carbonyl, dimethylaminocarbonyl, aminosulphonyl, methylaminosulphonyl and dimethylamino sulphonyl.

Generally, R³² represents halogen, carboxy, carboxy(C₁₋₆)alkyl, C₂₋₆ alkoxycarbonyl, C₂₋₆ alkoxycarbonyl(C₁₋₆)alkyl, a carboxylic acid isostere or prodrug moiety Ω, or —(C₁₋₆)alkyl-Ω.

Typically, R³² represents hydrogen, halogen, cyano, hydroxy, hydroxy(C₁₋₆)alkyl, C₁₋₆ alkylsulphonyl, formyl, carboxy, carboxy(C₁₋₆)alkyl, C₂₋₆ alkoxycarbonyl, C₂₋₆ alkoxycarbonyl(C₁₋₆)alkyl, aminosulphonyl, (C₁₋₆)alkylsulphoximinyl, [(C₁₋₆)alkyl][N—(C₁₋₆)alkyl]sulphoximinyl, (C₁₋₆)alkylsulphonylaminocarbonyl, (C₂₋₆)alkylcarbonylamino-sulphonyl, (C₁₋₆)alkoxyaminocarbonyl, tetrazolyl or hydroxyoxadiazolyl.

Typical values of R³² include hydrogen, fluoro, cyano, hydroxy, hydroxymethyl, methylsulphonyl, formyl, carboxy, carboxymethyl, carboxyethyl, methoxycarbonyl, ethoxycarbonyl, tert-butoxycarbonyl, methoxycarbonylmethyl, methoxycarbonylethyl, ethoxycarbonylmethyl, ethoxycarbonylethyl, aminosulphonyl, methylsulphoximinyl, (methyl)(N-methyl)sulphoximinyl, methylsulphonylaminocarbonyl, acetylaminosulphonyl, methoxyaminocarbonyl, tetrazolyl and hydroxyoxadiazolyl.

Suitably, R³² represents hydroxy.

Generally, R³³ represents hydrogen, halogen, C₁₋₆ alkyl or trifluoromethyl.

Suitably, R³³ represents hydrogen, C₁₋₆ alkyl or trifluoromethyl.

Selected values of R³³ include hydrogen, fluoro, methyl, ethyl, isopropyl, trifluoromethyl, hydroxy, hydroxymethyl, methoxy, amino and carboxy.

Particular values of R³³ include hydrogen, methyl, ethyl and trifluoromethyl.

In a first embodiment, R³³ represents hydrogen. In a second embodiment, R³³ represents halogen. In one aspect of that embodiment, R³³ represents fluoro. In a third embodiment, R³³ represents C₁₋₆ alkyl. In a first aspect of that embodiment, R³³ represents methyl. In a second aspect of that embodiment, R³³ represents ethyl. In a third aspect of that embodiment, R³³ represents isopropyl. In a fourth embodiment, R³³ represents trifluoromethyl. In a fifth embodiment, R³³ represents hydroxy. In a sixth embodiment, R³³ represents hydroxy(C₁₋₆)alkyl. In one aspect of that embodiment, R³³ represents hydroxymethyl. In a seventh embodiment, R³³ represents C₁₋₆ alkoxy. In one aspect of that embodiment, R³³ represents methoxy. In an eighth embodiment, R³³ represents amino. In a ninth embodiment, R³³ represents carboxy.

In a first embodiment, R³⁴ represents hydrogen. In a second embodiment, R³⁴ represents halogen. In one aspect of that embodiment, R³⁴ represents fluoro. In a third embodiment, R³⁴ represents halo(C₁₋₆)alkyl. In one aspect of that embodiment, R³⁴ represents fluoromethyl. In a fourth embodiment, R³⁴ represents hydroxy. In a fifth embodiment, R³⁴ represents C₁₋₆ alkoxy, especially methoxy. In a sixth embodiment, R³⁴ represents C₁₋₆ alkylthio, especially methylthio. In a seventh embodiment, R³⁴ represents C₁₋₆ alkylsulphinyl, especially methylsulphinyl. In an eighth embodiment, R³⁴ represents C₁₋₆ alkylsulphonyl, especially methylsulphonyl. In a ninth embodiment, R³⁴ represents amino. In a tenth embodiment, R³⁴ represents C₁₋₆ alkylamino, especially methylamino. In an eleventh embodiment, R³⁴ represents di(C₁₋₆)alkylamino, especially dimethylamino. In a twelfth embodiment, R³⁴ represents (C₂₋₆)alkylcarbonylamino, especially acetylamino. In a thirteenth embodiment, R³⁴ represents (C₂₋₆)alkylcarbonylamino(C₁₋₆)alkyl, especially acetylaminomethyl. In a fourteenth embodiment, R³⁴ represents (C₁₋₆)alkylsulphonyl-amino, especially methylsulphonylamino. In a fifteenth embodiment, R³⁴ represents (C₁₋₆)alkylsulphonylamino(C₁₋₆)alkyl, especially methylsulphonylaminomethyl.

Typically, R³⁴ represents hydrogen, halogen, halo(C₁₋₆)alkyl, hydroxy or (C₂₋₆)alkylcarbonylamino(C₁₋₆)alkyl.

Appositely, R³⁴ represents hydrogen, halogen, hydroxy or amino.

Suitably, R³⁴ represents hydrogen, halogen or hydroxy.

Selected values of R³⁴ include hydrogen, fluoro, fluoromethyl, hydroxy, methoxy, methylthio, methylsulphinyl, methylsulphonyl, amino, methylamino, dimethylamino and acetylaminomethyl.

Particular values of R³⁴ include hydrogen, fluoro, fluoromethyl, hydroxy and acetylaminomethyl.

Specific values of R³⁴ include hydrogen, fluoro, hydroxy and amino.

Suitably, R³⁴ represents hydrogen, fluoro or hydroxy.

Particular sub-groups of the compounds of formula (IIB-2) above are represented by the compounds of formula (IIC-2) and (IID-2) and N-oxides thereof, and pharmaceutically acceptable salts thereof:

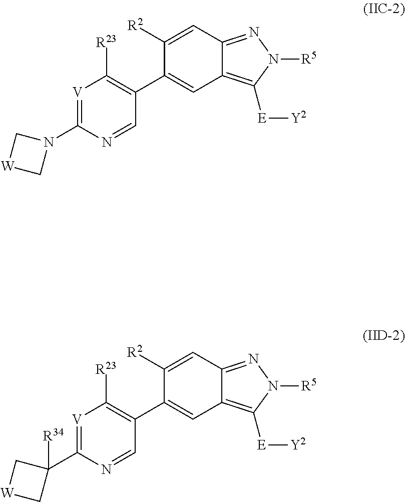

wherein

V, E, Y², W, R², R⁵, R²³ and R³⁴ are as defined above.

Specific novel compounds in accordance with the present invention include each of the compounds whose preparation is described in the accompanying Examples, and pharmaceutically acceptable salts thereof.

The compounds in accordance with the present invention are beneficial in the treatment and/or prevention of various human ailments. These include autoimmune and inflammatory disorders; neurological and neurodegenerative disorders; pain and nociceptive disorders; cardiovascular disorders; metabolic disorders; ocular disorders; and oncological disorders.

Inflammatory and autoimmune disorders include systemic autoimmune disorders, autoimmune endocrine disorders and organ-specific autoimmune disorders. Systemic autoimmune disorders include systemic lupus erythematosus (SLE), psoriasis, psoriatic arthropathy, vasculitis, polymyositis, scleroderma, multiple sclerosis, systemic sclerosis, ankylosing spondylitis, rheumatoid arthritis, non-specific inflammatory arthritis, juvenile inflammatory arthritis, juvenile idiopathic arthritis (including oligoarticular and polyarticular forms thereof), anaemia of chronic disease (ACD), Still's disease (juvenile and/or adult onset), Behçet's disease and Sjögren's syndrome. Autoimmune endocrine disorders include thyroiditis. Organ-specific autoimmune disorders include Addison's disease, haemolytic or pernicious anaemia, acute kidney injury (AKI; including cisplatin-induced AKI), diabetic nephropathy (DN), obstructive uropathy (including cisplatin-induced obstructive uropathy), glomerulonephritis (including Goodpasture's syndrome, immune complex-mediated glomerulonephritis and antineutrophil cytoplasmic antibodies (ANCA)-associated glomerulonephritis), lupus nephritis (LN), minimal change disease, Graves' disease, idiopathic thrombocytopenic purpura, inflammatory bowel disease (including Crohn's disease, ulcerative colitis, indeterminate colitis and pouchitis), pemphigus, atopic dermatitis, autoimmune hepatitis, primary biliary cirrhosis, autoimmune pneumonitis, autoimmune carditis, myasthenia gravis, spontaneous infertility, osteoporosis, osteopenia, erosive bone disease, chondritis, cartilage degeneration and/or destruction, fibrosing disorders (including various forms of hepatic and pulmonary fibrosis), asthma, rhinitis, chronic obstructive pulmonary disease (COPD), respiratory distress syndrome, sepsis, fever, muscular dystrophy (including Duchenne muscular dystrophy) and organ transplant rejection (including kidney allograft rejection).

Neurological and neurodegenerative disorders include Alzheimer's disease, Parkinson's disease, Huntington's disease, ischaemia, stroke, amyotrophic lateral sclerosis, spinal cord injury, head trauma, seizures and epilepsy.

Cardiovascular disorders include thrombosis, cardiac hypertrophy, hypertension, irregular contractility of the heart (e.g. during heart failure), and sexual disorders (including erectile dysfunction and female sexual dysfunction). Modulators of TNFα function may also be of use in the treatment and/or prevention of myocardial infarction (see J. J. Wu et al., JAMA, 2013, 309, 2043-2044).

Metabolic disorders include diabetes (including insulin-dependent diabetes mellitus and juvenile diabetes), dyslipidemia and metabolic syndrome.

Ocular disorders include retinopathy (including diabetic retinopathy, proliferative retinopathy, non-proliferative retinopathy and retinopathy of prematurity), macular oedema (including diabetic macular oedema), age-related macular degeneration (ARMD), vascularisation (including corneal vascularisation and neovascularisation), retinal vein occlusion, and various forms of uveitis and keratitis.

Oncological disorders, which may be acute or chronic, include proliferative disorders, especially cancer, and cancer-associated complications (including skeletal complications, cachexia and anaemia). Particular categories of cancer include haematological malignancy (including leukaemia and lymphoma) and non-haematological malignancy (including solid tumour cancer, sarcoma, meningioma, glioblastoma multiforme, neuroblastoma, melanoma, gastric carcinoma and renal cell carcinoma). Chronic leukaemia may be myeloid or lymphoid. Varieties of leukaemia include lymphoblastic T cell leukaemia, chronic myelogenous leukaemia (CML), chronic lymphocytic/lymphoid leukaemia (CLL), hairy-cell leukaemia, acute lymphoblastic leukaemia (ALL), acute myelogenous leukaemia (AML), myelodysplastic syndrome, chronic neutrophilic leukaemia, acute lymphoblastic T cell leukaemia, plasmacytoma, immunoblastic large cell leukaemia, mantle cell leukaemia, multiple myeloma, acute megakaryoblastic leukaemia, acute megakaryocytic leukaemia, promyelocytic leukaemia and erythroleukaemia. Varieties of lymphoma include malignant lymphoma, Hodgkin's lymphoma, non-Hodgkin's lymphoma, lymphoblastic T cell lymphoma, Burkitt's lymphoma, follicular lymphoma, MALT1 lymphoma and marginal zone lymphoma. Varieties of non-haematological malignancy include cancer of the prostate, lung, breast, rectum, colon, lymph node, bladder, kidney, pancreas, liver, ovary, uterus, cervix, brain, skin, bone, stomach and muscle. Modulators of TNFα function may also be used to increase the safety of the potent anticancer effect of TNF (see F. V. Hauwermeiren et al., J. Clin. Invest., 2013, 123, 2590-2603).

The present invention also provides a pharmaceutical composition which comprises a compound in accordance with the invention as described above, or a pharmaceutically acceptable salt or solvate thereof, in association with one or more pharmaceutically acceptable carriers.

Pharmaceutical compositions according to the invention may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

Preparations for oral administration may be suitably formulated to give controlled release of the active compound.

For buccal administration, the compositions may take the form of tablets or lozenges formulated in conventional manner.

The compounds of formula (I) may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

In addition to the formulations described above, the compounds of formula (I) may also be formulated as a depot preparation. Such long-acting formulations may be administered by implantation or by intramuscular injection.

For nasal administration or administration by inhalation, the compounds according to the present invention may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

For topical administration the compounds of use in the present invention may be conveniently formulated in a suitable ointment containing the active component suspended or dissolved in one or more pharmaceutically acceptable carriers. Particular carriers include, for example, mineral oil, liquid petroleum, propylene glycol, polyoxyethylene, polyoxypropylene, emulsifying wax and water. Alternatively, the compounds of use in the present invention may be formulated in a suitable lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the compounds of use in the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration compounds may be formulated in an ointment such as petrolatum.

For rectal administration the compounds of use in the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

The quantity of a compound of use in the invention required for the prophylaxis or treatment of a particular condition will vary depending on the compound chosen and the condition of the patient to be treated. In general, however, daily dosages may range from around 10 ng/kg to 1000 mg/kg, typically from 100 ng/kg to 100 mg/kg, e.g. around 0.01 mg/kg to 40 mg/kg body weight, for oral or buccal administration, from around 10 ng/kg to 50 mg/kg body weight for parenteral administration, and from around 0.05 mg to around 1000 mg, e.g. from around 0.5 mg to around 1000 mg, for nasal administration or administration by inhalation or insufflation.

If desired, a compound in accordance with the present invention may be co-administered with another pharmaceutically active agent, e.g. an anti-inflammatory molecule.

The compounds of formula (I) above wherein E represents —CH(OH)— and Y represents Y¹ may be prepared by a process which comprises reacting a compound of formula Y¹—CHO with a compound of formula (III):

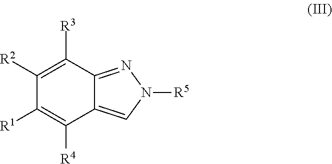

wherein Y¹, R¹, R², R³, R⁴ and R⁵ are as defined above.

The reaction will generally be accomplished in the presence of a base, typically a strong organic base such as lithium diisopropylamide. The reaction may conveniently be effected at ambient temperature in a suitable solvent, e.g. a cyclic ether such as tetrahydrofuran.

The compounds of formula (I) above wherein Y represents Y² may be prepared by a process which comprises reacting a compound of formula Y²—H with a compound of formula (IV):

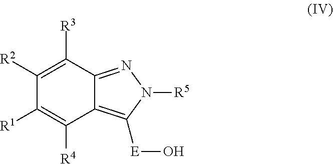

wherein E, Y², R¹, R², R³, R⁴ and R⁵ are as defined above.

The procedure is suitably effected in the presence of triphenylphosphine and a C₁₋₆ alkyl ester of azodicarboxylic acid, e.g. diisopropyl azodicarboxylate. Alternatively, the procedure may be effected in the presence of (cyanomethylene)tributylphosphorane or (tributyl-λ⁵-phosphanylidene)acetonitrile. The reaction is conveniently carried out in a suitable solvent, e.g. a cyclic ether such as tetrahydrofuran, or a chlorinated solvent such as dichloromethane, or an organic nitrile such as acetonitrile, or an aromatic hydrocarbon such as toluene.

Alternatively, the procedure may be effected in the presence of a sulphonic acid derivative, e.g. a C₁₋₆ alkylsulphonic acid such as methanesulphonic acid. The reaction is conveniently carried out at an elevated temperature in a suitable solvent, e.g. a cyclic ether such as 1,4-dioxane.

In an alternative procedure, the compounds of formula (I) above wherein Y represents Y² may be prepared by a process which comprises reacting a compound of formula Y²—H with a compound of formula (V):

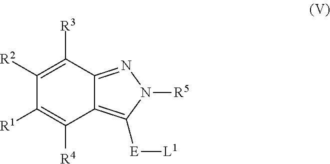

wherein E, Y², R¹, R², R³, R⁴ and R⁵ are as defined above, and L¹ represents a suitable leaving group.

The leaving group L¹ is suitably a halogen atom, e.g. chloro; or a sulphonate derivative, e.g. a C₁₋₆ alkylsulphonate such as methylsulphonate.

Where L¹ is halo, the procedure is suitably effected in the presence of a base, e.g. an alkali metal carbonate such as cesium carbonate or potassium carbonate. The reaction is conveniently carried out at ambient or elevated temperature in a suitable solvent, e.g. a dipolar aprotic solvent such as N,N-dimethylformamide or N,N-dimethylacetamide.

Where L¹ is a sulphonate derivative, e.g. methylsulphonate, the procedure is suitably effected in the presence of a base, e.g. an alkali metal hydride such as sodium hydride. The reaction is conveniently carried out at an elevated temperature in a suitable solvent, e.g. a dipolar aprotic solvent such as N,N-dimethylformamide.

The intermediates of formula (V) wherein L¹ is chloro may be prepared from the corresponding compound of formula (IV) by treatment with a chlorinating agent such as thionyl chloride. The reaction is conveniently carried out in a suitable solvent, e.g. a cyclic ether such as tetrahydrofuran, or a chlorinated solvent such as dichloromethane.

The intermediates of formula (V) wherein L¹ is methylsulphonate may be prepared from the corresponding compound of formula (IV) by treatment with methanesulphonic anhydride, typically in the presence of a base, e.g. an alkali metal hydride such as sodium hydride. The reaction is conveniently carried out at an elevated temperature in a suitable solvent, e.g. a dipolar aprotic solvent such as N,N-dimethylformamide.

The intermediates of formula (IV) above wherein E is methylene may be prepared by reducing a compound of formula (VI):

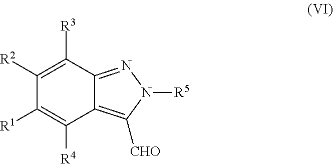

wherein R¹, R², R³, R⁴ and R⁵ are as defined above.

The procedure is suitably effected by contacting compound (VI) with a reducing agent, e.g. sodium borohydride. The reaction is conveniently carried out in a suitable solvent, e.g. a C₁₋₄ alkanol such as methanol.

Where they are not commercially available, the starting materials of formula (III) and (VI) may be prepared by methods analogous to those described in the accompanying Examples, or by standard methods well known from the art.

It will be understood that any compound of formula (I) initially obtained from any of the above processes may, where appropriate, subsequently be elaborated into a further compound of formula (I) by techniques known from the art. By way of example, a compound of formula (I) wherein E represents —CH(OH)— may be converted into the corresponding compound wherein E represents —CH₂— by heating with elemental iodine and phosphinic acid in acetic acid; or by treating with triethylsilane and an acid, e.g. an organic acid such as trifluoroacetic acid, or a Lewis acid such as boron trifluoride diethyl etherate; or by treating with chlorotrimethylsilane and sodium iodide; or by a two-step procedure which comprises: (i) treatment with thionyl bromide; and (ii) treatment of the product thereby obtained with a transition metal catalyst, e.g. (2,2′-bipyridine)dichloro-ruthenium(II) hydrate, in the presence of diethyl 1,4-dihydro-2,6-dimethyl-3,5-pyridine-dicarboxylate (Hantzsch ester) and a base, e.g. an organic base such as N,N-diisopropyl-ethylamine.

A compound of formula (I) wherein E represents —C(O)— may be converted into the corresponding compound wherein E represents —CH(OH)— by treatment with a reducing agent such as sodium borohydride.

A compound of formula (I) wherein E represents —CH₂— may be converted into the corresponding compound wherein E represents —CH(CH₃)— by treatment with a methyl halide, e.g. methyl iodide, in the presence of a base such as lithium hexamethyldisilazide.

A compound of formula (I) which contains a hydroxy group may be alkylated by treatment with the appropriate alkyl halide in the presence of a base, e.g. sodium hydride, or silver oxide. A compound of formula (I) which contains hydroxy may be converted into the corresponding fluoro-substituted compound by treatment with diethylaminosulfur trifluoride (DAST) or bis(2-methoxyethyl)aminosulfur trifluoride (BAST). A compound of formula (I) which contains hydroxy may be converted into the corresponding difluoro-substituted compound via a two-step procedure which comprises: (i) treatment with an oxidising agent, e.g. manganese dioxide; and (ii) treatment of the carbonyl-containing compound thereby obtained with DAST.

A compound of formula (I) which contains an N—H moiety may be alkylated by treatment with the appropriate alkyl halide, typically at an elevated temperature in an organic solvent such as acetonitrile; or at ambient temperature in the presence of a base, e.g. an alkali metal carbonate such as potassium carbonate or cesium carbonate, in a suitable solvent, e.g. a dipolar aprotic solvent such as N,N-dimethylformamide. Alternatively, a compound of formula (I) which contains an N—H moiety may be alkylated by treatment with the appropriate alkyl tosylate in the presence of a base, e.g. an inorganic base such as sodium hydride, or an organic base such as 1,8-diazabicyclo[5.4.0]undec-7-ene (DBU).

A compound of formula (I) which contains an N—H moiety may be methylated by treatment with formaldehyde in the presence of a reducing agent, e.g. sodium triacetoxyborohydride.

A compound of formula (I) which contains an N—H moiety may be acylated by treatment with the appropriate acid chloride, e.g. acetyl chloride, or with the appropriate carboxylic acid anhydride, e.g. acetic anhydride, typically at ambient temperature in the presence of a base, e.g. an organic base such as triethylamine.

A compound of formula (I) which contains an N—H moiety may be converted into the corresponding compound wherein the nitrogen atom is substituted by C₁₋₆ alkyl-sulphonyl, e.g. methylsulphonyl, by treatment with the appropriate C₁₋₆ alkylsulphonyl chloride, e.g. methanesulphonyl chloride, or with the appropriate C₁₋₆ alkylsulphonic acid anhydride, e.g. methanesulphonic anhydride, typically at ambient temperature in the presence of a base, e.g. an organic base such as triethylamine or N,N-diisopropylethyl-amine.

A compound of formula (I) substituted by amino (—NH₂) may be converted into the corresponding compound substituted by C₁₋₆ alkylsulphonylamino, e.g. methylsulphonyl-amino, or bis[(C₁₋₆)alkylsulphonyl]amino, e.g. bis(methylsulphonyl)amino, by treatment with the appropriate C₁₋₆ alkylsulphonyl halide, e.g. a C₁₋₆ alkylsulphonyl chloride such as methanesulphonyl chloride. Similarly, a compound of formula (I) substituted by hydroxy (—OH) may be converted into the corresponding compound substituted by C₁₋₆ alkyl-sulphonyloxy, e.g. methylsulphonyloxy, by treatment with the appropriate C₁₋₆ alkyl-sulphonyl halide, e.g. a C₁₋₆ alkylsulphonyl chloride such as methanesulphonyl chloride.

A compound of formula (I) containing the moiety —S— may be converted into the corresponding compound containing the moiety —S(O)— by treatment with 3-chloroperoxy-benzoic acid. Likewise, a compound of formula (I) containing the moiety —S(O)— may be converted into the corresponding compound containing the moiety —S(O)₂— by treatment with 3-chloroperoxybenzoic acid. Alternatively, a compound of formula (I) containing the moiety —S— may be converted into the corresponding compound containing the moiety —S(O)₂— by treatment with Oxone® (potassium peroxymonosulfate).

A compound of formula (I) containing an aromatic nitrogen atom may be converted into the corresponding N-oxide derivative by treatment with 3-chloroperoxy-benzoic acid.

A bromophenyl derivative of formula (I) may be converted into the corresponding optionally substituted 2-oxopyrrolidin-1-ylphenyl or 2-oxooxazolidin-3-ylphenyl derivative by treatment with pyrrolidin-2-one or oxazolidin-2-one, or an appropriately substituted analogue thereof. The reaction is conveniently effected at an elevated temperature in the presence of copper(I) iodide, trans-N,N′-dimethylcyclohexane-1,2-diamine and an inorganic base such as potassium carbonate.

A compound of formula (I) wherein R¹ represents halogen, e.g. bromo, may be converted into the corresponding compound wherein R¹ represents an optionally substituted aryl or heteroaryl moiety by treatment with the appropriately substituted aryl or heteroaryl boronic acid or a cyclic ester thereof formed with an organic diol, e.g. pinacol, 1,3-propanediol or neopentyl glycol. The reaction is typically effected in the presence of a transition metal catalyst, e.g. [1,1′-bis(diphenylphosphino)ferrocene]dichloropalladium(II), tetrakis(triphenylphosphine)palladium(0), or bis[3-(diphenylphosphanyl)cyclopenta-2,4-dien-1-yl]iron-dichloropalladium-dichloromethane complex, and a base, e.g. an inorganic base such as sodium carbonate or potassium carbonate, or potassium phosphate.

A compound of formula (I) wherein R¹ represents halogen, e.g. bromo, may be converted into the corresponding compound wherein R¹ represents an optionally substituted aryl, heteroaryl or heterocycloalkenyl moiety via a two-step procedure which comprises: (i) reaction with bis(pinacolato)diboron or bis(neopentyl glycolato)diboron; and (ii) reaction of the compound thereby obtained with an appropriately functionalised halo- or tosyloxy-substituted aryl, heteroaryl or heterocycloalkenyl derivative. Step (i) is conveniently effected in the presence of a transition metal catalyst such as [1,1′-bis-(diphenylphosphino)ferrocene]dichloropalladium(II), or bis[3-(diphenylphosphanyl)-cyclopenta-2,4-dien-1-yl]iron-dichloropalladium-dichloromethane complex. Step (ii) is conveniently effected in the presence of a transition metal catalyst such as tetrakis-(triphenylphosphine)palladium(0), or bis[3-(diphenylphosphanyl)cyclopenta-2,4-dien-1-yl]iron-dichloropalladium-dichloromethane complex, and a base, e.g. an inorganic base such as sodium carbonate or potassium carbonate.

A compound of formula (I) wherein R¹ represents halogen, e.g. bromo, may be converted into the corresponding compound wherein R¹ represents an optionally substituted C₂₋₆ alkynyl moiety by treatment with an appropriately substituted alkyne derivative, e.g. 2-hydroxybut-3-yne. The reaction is conveniently accomplished with the assistance of a transition metal catalyst, e.g. tetrakis(triphenylphosphine)palladium(0), typically in the presence of copper(I) iodide and a base, e.g. an organic base such as triethylamine.

A compound of formula (I) wherein R¹ represents halogen, e.g. bromo, may be converted into the corresponding compound wherein R¹ represents an optionally substituted imidazol-1-yl moiety by treatment with the appropriately substituted imidazole derivative, typically in the presence of copper(II) acetate and an organic base such as N,N,N′,N′-tetramethylethylenediamine (TMEDA).

A compound of formula (I) wherein R¹ represents halogen, e.g. bromo, may be converted into the corresponding compound wherein R¹ represents 2-(methoxycarbonyl)-ethyl via a two-step procedure which comprises: (i) reaction with methyl acrylate; and (ii) catalytic hydrogenation of the alkenyl derivative thereby obtained, typically by treatment with a hydrogenation catalyst, e.g. palladium on charcoal, under an atmosphere of hydrogen gas. Step (i) is typically effected in the presence of a transition metal catalyst, e.g. palladium(II) acetate or bis(dibenzylideneacetone)palladium(0), and a reagent such as tri(ortho-tolyl)phosphine.

In general, a compound of formula (I) containing a —C═C— functionality may be converted into the corresponding compound containing a —CH—CH— functionality by catalytic hydrogenation, typically by treatment with a hydrogenation catalyst, e.g. palladium on charcoal, under an atmosphere of hydrogen gas, optionally in the presence of a base, e.g. an alkali metal hydroxide such as sodium hydroxide.

A compound of formula (I) wherein R¹ represents 6-methoxypyridin-3-yl may be converted into the corresponding compound wherein R¹ represents 2-oxo-1,2-dihydro-pyridin-5-yl by treatment with pyridine hydrochloride; or by heating with a mineral acid such as hydrochloric acid. By utilising similar methodology, a compound of formula (I) wherein R¹ represents 6-methoxy-4-methylpyridin-3-yl may be converted into the corresponding compound wherein R¹ represents 4-methyl-2-oxo-1,2-dihydropyridin-5-yl; and a compound of formula (I) wherein R¹ represents 6-methoxy-5-methylpyridin-3-yl may be converted into the corresponding compound wherein R¹ represents 3-methyl-2-oxo-1,2-dihydropyridin-5-yl.

A compound of formula (I) wherein R¹ represents 2-oxo-1,2-dihydropyridin-5-yl may be converted into the corresponding compound wherein R¹ represents 2-oxopiperidin-5-yl by catalytic hydrogenation, typically by treatment with gaseous hydrogen in the presence of a hydrogenation catalyst such as platinum(IV) oxide.

A compound of formula (I) containing an ester moiety, e.g. a C₂₋₆ alkoxycarbonyl group such as methoxycarbonyl or ethoxycarbonyl, may be converted into the corresponding compound containing a carboxy (—CO₂H) moiety by treatment with an acid, e.g. a mineral acid such as hydrochloric acid.

A compound of formula (I) containing an N-(tert-butoxycarbonyl) moiety may be converted into the corresponding compound containing an N—H moiety by treatment with an acid, e.g. a mineral acid such as hydrochloric acid, or an organic acid such as trifluoroacetic acid.

A compound of formula (I) containing an ester moiety, e.g. a C₂₋₆ alkoxycarbonyl group such as methoxycarbonyl or ethoxycarbonyl, may alternatively be converted into the corresponding compound containing a carboxy (—CO₂H) moiety by treatment with a base, e.g. an alkali metal hydroxide selected from lithium hydroxide, sodium hydroxide and potassium hydroxide; or an organic base such as sodium methoxide or sodium ethoxide.

A compound of formula (I) containing a carboxy (—CO₂H) moiety may be converted into the corresponding compound containing an amide moiety by treatment with the appropriate amine in the presence of a condensing agent such as 1-ethyl-3-(3-dimethyl-aminopropyl)carbodiimide.

A compound of formula (I) containing a carbonyl (C═O) moiety may be converted into the corresponding compound containing a —C(CH₃)(OH)— moiety by treatment with methylmagnesium bromide. Similarly, a compound of formula (I) containing a carbonyl (C═O) moiety may be converted into the corresponding compound containing a —C(CF₃)(OH)— moiety by treatment with (trifluoromethyl)trimethylsilane and cesium fluoride. A compound of formula (I) containing a carbonyl (C═O) moiety may be converted into the corresponding compound containing a —C(CH₂NO₂)(OH)— moiety by treatment with nitromethane.

A compound of formula (I) containing a hydroxymethyl moiety may be converted into the corresponding compound containing a formyl (—CHO) moiety by treatment with an oxidising agent such as Dess-Martin periodinane. A compound of formula (I) containing a hydroxymethyl moiety may be converted into the corresponding compound containing a carboxy moiety by treatment with an oxidising agent such as tetrapropylammonium perruthenate.

A compound of formula (I) wherein R¹ represents a substituent containing at least one nitrogen atom, which substituent is linked to the remainder of the molecule via a nitrogen atom, may be prepared by reacting a compound of formula (I) wherein R¹ represents halogen, e.g. bromo, with the appropriate compound of formula R¹—H [e.g. 1-(pyridin-3-yl)piperazine or morpholine]. The reaction is conveniently effected with the assistance of a transition metal catalyst, e.g. tris(dibenzylideneacetone)dipalladium(0), in the presence of an amination ligand such as 2-dicyclohexylphosphino-2′,4′,6′-triisopropyl-biphenyl (XPhos) or 2,2′-bis(diphenylphosphino)-1,1′-binaphthalene (BINAP) and a base, e.g. an inorganic base such as sodium tert-butoxide. Alternatively, the reaction may be effected using palladium diacetate, in the presence of a reagent such as [2′,6′-bis(propan-2-yloxy)biphenyl-2-yl](dicyclohexyl)phosphane and a base, e.g. an inorganic base such as cesium carbonate.

A compound of formula (I) containing an oxo moiety can be converted into the corresponding compound containing an ethoxycarbonylmethylidene moiety by treatment with triethyl phosphonoacetate in the presence of a base such as sodium hydride.

A compound of formula (IIB) wherein R²¹ represents ethenyl may be prepared by reacting a compound of formula (IIB) wherein R²¹ represents halogen, e.g. chloro, with potassium vinyl trifluoroborate. The reaction is typically effected in the presence of a transition metal catalyst, e.g. [1,1′-bis(diphenylphosphino)ferrocene]dichloropalladium(II), and a base, e.g. an organic base such as triethylamine.

A compound of formula (IIB) wherein R²¹ represents halogen, e.g. chloro, may be converted into the corresponding compound wherein R²¹ represents an optionally substituted C₄₋₇ cycloalkenyl moiety by treatment with the appropriately substituted cycloalkenyl boronic acid or a cyclic ester thereof formed with an organic diol, e.g. pinacol, 1,3-propanediol or neopentyl glycol. The reaction is typically effected in the presence of a transition metal catalyst, e.g. bis[3-(diphenylphosphanyl)cyclopenta-2,4-dien-1-yl]iron-dichloropalladium-dichloromethane complex, and a base, e.g. an inorganic base such as potassium carbonate.

A compound of formula (IIB) wherein R²¹ represents a substituent containing at least one nitrogen atom, which substituent is linked to the remainder of the molecule via a nitrogen atom, may be prepared by reacting a compound of formula (IIB) wherein R²¹ represents halogen, e.g. chloro, with the appropriate compound of formula R²¹—H [e.g. 2-methoxyethylamine, N-methyl-L-alanine, 2-aminocyclopentanecarboxylic acid, 3-aminocyclopentanecarboxylic acid, 1-(aminomethyl)cyclopropanecarboxylic acid, methyl azetidine-3-carboxylate, pyrrolidin-3-ol, pyrrolidine-3-carboxylic acid, piperidine-2-carboxylic acid, piperidine-3-carboxylic acid, 4-(1H-tetrazol-5-yl)piperidine, piperazine, 1-(methylsulfonyl)piperazine, piperazin-2-one, 2-(piperazin-1-yl)propanoic acid, morpholine, morpholine-2-carboxylic acid, thiomorpholine, thiomorpholine 1,1-dioxide, 1,4-diazepan-5-one, 2-oxa-5-azabicyclo[2.2.1]heptane or an appropriately substituted azaspiroalkane], optionally in the presence of a base, e.g. an organic base such as triethylamine or N,N-diisopropylethylamine and/or 1-methyl-2-pyrrolidinone, or pyridine, or an inorganic base such as potassium carbonate.

Where a mixture of products is obtained from any of the processes described above for the preparation of compounds according to the invention, the desired product can be separated therefrom at an appropriate stage by conventional methods such as preparative HPLC; or column chromatography utilising, for example, silica and/or alumina in conjunction with an appropriate solvent system.

Where the above-described processes for the preparation of the compounds according to the invention give rise to mixtures of stereoisomers, these isomers may be separated by conventional techniques. In particular, where it is desired to obtain a particular enantiomer of a compound of formula (I) this may be produced from a corresponding mixture of enantiomers using any suitable conventional procedure for resolving enantiomers. Thus, for example, diastereomeric derivatives, e.g. salts, may be produced by reaction of a mixture of enantiomers of formula (I), e.g. a racemate, and an appropriate chiral compound, e.g. a chiral base. The diastereomers may then be separated by any convenient means, for example by crystallisation, and the desired enantiomer recovered, e.g. by treatment with an acid in the instance where the diastereomer is a salt. In another resolution process a racemate of formula (I) may be separated using chiral HPLC. Moreover, if desired, a particular enantiomer may be obtained by using an appropriate chiral intermediate in one of the processes described above. Alternatively, a particular enantiomer may be obtained by performing an enantiomer-specific enzymatic biotransformation, e.g. an ester hydrolysis using an esterase, and then purifying only the enantiomerically pure hydrolysed acid from the unreacted ester antipode. Chromatography, recrystallisation and other conventional separation procedures may also be used with intermediates or final products where it is desired to obtain a particular geometric isomer of the invention.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3^{rd} edition, 1999. The protecting groups may be removed at any convenient subsequent stage utilising methods known from the art.

The following Examples illustrate the preparation of compounds according to the invention.

The compounds in accordance with the present invention potently inhibit the binding of a fluorescence conjugate to TNFα when tested in the fluorescence polarisation assay described herein. Indeed, when tested in that assay, the compounds of the present invention exhibit an IC₅₀ value of 50 μM or less, generally of 20 μM or less, usually of 5 μM or less, typically of 1 μM or less, suitably of 500 nM or less, ideally of 100 nM or less, and preferably of 20 nM or less (the skilled person will appreciate that a lower IC₅₀ figure denotes a more active compound).

Certain compounds in accordance with the present invention potently neutralise the activity of TNFα in a commercially available HEK-293 derived reporter cell line known as HEK-Blue™ CD40L. This is a stable HEK-293 transfected cell line expressing SEAP (secreted embryonic alkaline phosphatase) under the control of the IFNβ minimal promoter fused to five NF-κB binding sites. Secretion of SEAP by these cells is stimulated in a concentration-dependent manner by TNFα. When tested in the HEK-293 bioassay, also referred to herein as the reporter gene assay, certain compounds of the present invention exhibit an IC₅₀ value of 50 μM or less, generally of 20 μM or less, usually of 5 μM or less, typically of 1 μM or less, suitably of 500 nM or less, ideally of 100 nM or less, and preferably of 20 nM or less (as before, the skilled person will appreciate that a lower IC₅₀ figure denotes a more active compound).

### Fluorescence Polarisation Assay

### Preparation of Compound (A)

1-(2,5-Dimethylbenzyl)-6-[4-(piperazin-1-ylmethyl)phenyl]-2-(pyridin-4-yl-methyl)-1H-benzimidazole—hereinafter referred to as “Compound (A)”—can be prepared by the procedure described in Example 499 of WO 2013/186229; or by a procedure analogous thereto.

### Preparation of Fluorescence Conjugate

Compound (A) (27.02 mg, 0.0538 mmol) was dissolved in DMSO (2 mL). 5 (−6) Carboxy-fluorescein succinimyl ester (24.16 mg, 0.0510 mmol) (Invitrogen catalogue number: C1311) was dissolved in DMSO (1 mL) to give a bright yellow solution. The two solutions were mixed at room temperature, the mixture turning red in colour. The mixture was stirred at room temperature. Shortly after mixing a 20 μL aliquot was removed and diluted in a 80:20 mixture of AcOH:H₂O for LC-MS analysis on the 1200RR-6140 LC-MS system. The chromatogram showed two closely eluting peaks at retention times of 1.42 and 1.50 minutes, both with mass (M+H)⁺=860.8 amu, corresponding to the two products formed with the 5- and 6-substituted carboxyfluorescein group. A further peak at retention time 2.21 minutes had a mass of (M+H)⁺=502.8 amu, corresponding to Compound (A). No peak was observed for unreacted 5(−6) carboxyfluorescein succinimyl ester. The peak areas were 22.0%, 39.6% and 31.4% for the three signals, indicating a 61.6% conversion to the two isomers of the desired fluorescence conjugate at that time-point. Further 20 μL aliquots were extracted after several hours and then after overnight stirring, diluted as before and subjected to LC-MS analysis. The percentage conversion was determined as 79.8% and 88.6% respectively at these time-points. The mixture was purified on a UV-directed preparative HPLC system. The pooled purified fractions were freeze-dried to remove excess solvent. After freeze-drying, an orange solid (23.3 mg) was recovered, equivalent to 0.027 mmol of fluorescence conjugate, corresponding to an overall yield of 53% for the reaction and preparative HPLC purification.

### Inhibition of Binding of Fluorescence Conjugate to TNFα

Compounds were tested at 10 concentrations starting from 25 μM in a final assay concentration of 5% DMSO, by pre-incubation with TNFα for 60 minutes at ambient temperature in 20 mM Tris, 150 mM NaCl, 0.05% Tween 20, before addition of the fluorescence conjugate and a further incubation for 20 hours at ambient temperature. The final concentrations of TNFα and the fluorescence conjugate were 10 nM and 10 nM respectively in a total assay volume of 25 μL. Plates were read on a plate reader capable of detecting fluorescence polarisation (e.g. an Analyst HT plate reader; or an Envision plate reader). An IC₅₀ value was calculated using XLfit™ (4 parameter logistic model) in ActivityBase.

When tested in the fluorescence polarisation assay, the compounds of the accompanying Examples were all found to exhibit IC₅₀ values of 50 μM or better.

Thus, when tested in the fluorescence polarisation assay, compounds of the accompanying Examples exhibit IC₅₀ values generally in the range of about 0.01 nM to about 50 μM, usually in the range of about 0.01 nM to about 20 μM, typically in the range of about 0.01 nM to about 5 μM, suitably in the range of about 0.01 nM to about 1 μM, appositely in the range of about 0.01 nM to about 500 nM, ideally in the range of about 0.01 nM to about 100 nM, and preferably in the range of about 0.01 nM to about 25 nM.

### Reporter Gene Assay

### Inhibition of TNFα-Induced NF-κB Activation

Stimulation of HEK-293 cells by TNFα leads to activation of the NF-κB pathway. The reporter cell line used to determine TNFα activity was purchased from InvivoGen. HEK-Blue™ CD40L is a stable HEK-293 transfected cell line expressing SEAP (secreted embryonic alkaline phosphatase) under the control of the IFNβ minimal promoter fused to five NF-κB binding sites. Secretion of SEAP by these cells is stimulated in a dose-dependent manner by TNFα, with an EC50 of 0.5 ng/mL for human TNFα. Compounds were diluted from 10 mM DMSO stocks (final assay concentration 0.3% DMSO) to generate a 10-point 3-fold serial dilution curve (e.g. 30,000 nM to 2 nM final concentration). Diluted compound was preincubated with TNFα for 60 minutes prior to addition to a 384-well microtitre plate and incubated for 18 h. The final TNFα concentration in the assay plate was 0.5 ng/mL. SEAP activity was determined in the supernatant using a colorimetric substrate, e.g. QUANTI-Blue™ or HEK-Blue™ Detection media (InvivoGen). Percentage inhibitions for compound dilutions were calculated between a DMSO control and maximum inhibition (by excess control compound) and an IC₅₀ value calculated using XLfit™ (4 parameter logistic model) in ActivityBase.

When tested in the reporter gene assay, certain compounds of the accompanying Examples were found to exhibit IC₅₀ values of 50 μM or better.

Thus, when tested in the reporter gene assay, compounds of the accompanying Examples exhibit IC₅₀ values generally in the range of about 0.01 nM to about 50 μM, usually in the range of about 0.01 nM to about 20 μM, typically in the range of about 0.01 nM to about 5 μM, suitably in the range of about 0.01 nM to about 1 μM, appositely in the range of about 0.01 nM to about 500 nM, ideally in the range of about 0.01 nM to about 100 nM, and preferably in the range of about 0.01 nM to about 25 nM.

### EXAMPLES

### Abbreviations

DCM: dichloromethane EtOAc: ethyl acetate
DMSO: dimethylsulfoxide THF: tetrahydrofuran
MeOH: methanol DMF: N,N-dimethylformamide
TBAF: tetrabutylammonium fluoride
Dess-Martin periodinane: 1,1,1-tris(acetyloxy)-1,1-dihydro-1,2-benziodoxol-3-(1H)-one
Pd(dppf)Cl₂: [1,1′-bis(diphenylphosphino)ferrocene]dichloropalladium(II)
h: hour M: mass

### LCMS: Liquid Chromatography Mass Spectrometry

RT: retention time

### Nomenclature

Compounds were named with the aid of ACD/Name Batch (Network) version 11.01, and/or Accelrys Draw 4.0.

### Analytical Conditions

### HPLC

Method D (uPLC)
Column: Phenomenex Kinetex-XB C18 (2.1×100 mm, 1.7 μm column)
Flow rate: 0.6 mL/min
Solvent A: 0.1% formic acid/water
Solvent B: 0.1% formic acid/acetonitrile
Injection volume: 3 μL
Column temperature: 40° C.
UV detection wavelength: 215 nm
Eluent: 0 to 5.3 minutes, constant gradient from 95% solvent A+5% solvent B to 100% solvent B; 5.3 to 5.8 minutes, 100% solvent B; 5.80 to 5.82 minutes, constant gradient from 100% solvent B to 95% solvent A+5% solvent B.
MS detection using Waters LCT or LCT Premier or ZQ or ZMD.
UV detection using Waters 2996 photodiode array or Waters 2787 UV or Waters 2788 UV.

### Intermediate 1

### (5-Bromo-2-methyl-2H-indazol-3-yl)methanol

A solution of 5-bromo-2-methyl-2H-indazole-3-carbaldehyde (1.5 g, 6.27 mmol) in methanol (35 mL) was cooled to 0° C. (ice/methanol bath) with stirring under nitrogen. Sodium borohydride (265 mg, 7 mmol) was added portionwise over 10 minutes. The resulting mixture was stirred at 0° C. for 30 minutes, then allowed to warm to ambient temperature and stirred for 1 h. The reaction mixture was quenched by the addition of excess crushed ice. The crude mixture was concentrated in vacuo and the residue was partitioned between ethyl acetate (50 mL) and water (50 mL). The aqueous layer was separated and washed with further ethyl acetate (30 mL). The combined organic layers were dried over MgSO₄ and concentrated in vacuo to afford the title compound (1.36 g, 90%) as a yellow solid. δ_{H} (500 MHz, CDCl₃) 7.77 (dd, J 1.8, 0.6 Hz, 1H), 7.53 (dd, J 9.1, 0.6 Hz, 1H), 7.32 (dd, J 9.1, 1.8 Hz, 1H), 5.00 (d, J 5.1 Hz, 2H), 4.20 (s, 3H), 1.97 (t, J 5.6 Hz, 1H). HPLC-MS: MH⁺ m/z 243.

### Intermediate 2

### Ethyl (2R)-2-(2-fluoro-6-nitrophenoxy)propanoate

2-Fluoro-6-nitrophenol (2.5 g, 15.91 mmol), ethyl (2S)-2-hydroxypropanoate (1.82 mL, 15.91 mmol) and triphenylphosphine (3.83 mL, 17.5 mmol) were dissolved in DCM (10 mL). The reaction mixture was cooled to 0° C. and diisopropyl azodicarboxylate (3.67 mL, 17.5 mmol) was added slowly dropwise. The reaction mixture was allowed to warm to ambient temperature and stirred for 1.5 h. The reaction mixture was diluted with water (20 mL) and DCM (75 mL). The aqueous layer was further extracted with DCM (50 mL), then the combined organic layers were washed sequentially with saturated aqueous sodium hydrogen carbonate solution (40 mL) and brine (40 mL). The organic layer was dried over Na₂SO₄ and the solvent was removed in vacuo. The residue was purified on silica, eluting with 0-100% ethyl acetate in heptane, to afford the title compound (2.8 g, 83%) as a yellow oil. δ_{H} (500 MHz, CDCl₃) 7.62 (dt, J 8.2, 1.5 Hz, 1H), 7.34 (ddd, J 11.0, 8.4, 1.5 Hz, 1H), 7.16 (td, J 8.3, 4.9 Hz, 1H), 4.93 (q, J 6.8 Hz, 1H), 4.25-4.17 (m, 2H), 1.70 (d, J 6.8 Hz, 3H), 1.26 (t, J 7.1 Hz, 3H).

### Intermediate 3

### (2R)-8-Fluoro-2-methyl-3,4-dihydro-2H-1,4-benzoxazin-3-one

Intermediate 2 (3.4 g, 13.22 mmol) was dissolved in ethanol (40 mL) and water (10 mL). Iron powder (2.21 g, 39.66 mmol) and 11M aqueous hydrogen chloride solution (0.12 mL) were added and the reaction mixture was stirred at 80° C. for 2 h. The reaction mixture was allowed to cool to ambient temperature and adjusted to pH 8 with 5M aqueous NaOH solution, then diluted with dichloromethane (50 mL) and methanol (50 mL). The reaction mixture was filtered through a pad of Celite and the filtrate was concentrated in vacuo. The residue was dissolved in DCM (50 mL), then washed sequentially with saturated aqueous sodium hydrogen carbonate solution (30 mL) and brine (30 mL). The organic layer was dried over Na₂SO₄ and concentrated in vacuo to afford the title compound (2.05 g, 86%) as a beige solid. δ_{H} (500 MHz, CDCl₃) 8.14 (s, 1H), 6.92 (td, J 8.2, 5.2 Hz, 1H), 6.84 (ddd, J 9.9, 8.4, 1.3 Hz, 1H), 6.63-6.59 (m, 1H), 4.75 (q, J 6.8 Hz, 1H), 1.66 (d, J 6.8 Hz, 3H).

### Intermediate 4

### (2R)-4-[(5-Bromo-2-methyl-2H-indazol-3-yl)methyl]-8-fluoro-2-methyl-3,4-dihydro-2H-1,4-benzoxazin-3-one

A mixture of Intermediate 3 (350 mg, 1.93 mmol), Intermediate 1 (510 mg, 2.12 mmol) and triphenylphosphine (610 mg, 2.33 mmol) in anhydrous tetrahydrofuran (15 mL) was cooled to −25° C. with stirring under nitrogen. Diisopropyl azodicarboxylate (0.46 mL, 2.34 mmol) was added dropwise. The reaction mixture was allowed to warm to ambient temperature and stirred for 18 h. The reaction mixture was diluted with ethyl acetate (30 mL), then washed sequentially with saturated aqueous sodium bicarbonate solution (30 mL) and brine (30 mL). The organic layer was separated and concentrated in vacuo. The resulting brown oil was purified by chromatography on silica gel, eluting with a gradient of 0-40% ethyl acetate in heptane. The title compound (357 mg, 35%) was isolated as a pale yellow solid. δ_{H} (500 MHz, CDCl₃) 7.69-7.66 (m, 1H), 7.53 (d, J 9.1 Hz, 1H), 7.31 (dd, J 9.1, 1.8 Hz, 1H), 6.88-6.81 (m, 2H), 6.74-6.70 (m, 1H), 5.58 (d, J 16.4 Hz, 1H), 5.43 (d, J 16.4 Hz, 1H), 4.79 (q, J 6.8 Hz, 1H), 4.15 (s, 3H), 1.67 (d, J 6.8 Hz, 3H). HPLC-MS: MH⁺ m/z 404.

### Intermediate 5

### 5-Bromo-2-(methanesulfinyl)pyridine

NaIO₄ (9.56 g, 44.69 mmol) was added as a slurry in water (10 mL) to a stirred solution of 5-bromo-2-(methylsulfanyl)pyridine (2.4 g, 11.76 mmol) in acetic acid (40 mL) at room temperature. The mixture was stirred at room temperature for 2 h. After this time, a colourless precipitate had formed. The mixture was treated with water (50 mL), upon which the precipitate dissolved. The aqueous acidic mixture was basified through addition of saturated aqueous potassium carbonate solution and the resulting material was extracted with EtOAc (3×50 mL). The combined organic phase was washed with 10% aqueous sodium thiosulfate solution (50 mL), then dried (Na₂SO₄) and reduced in vacuo. The resulting crude amber glass (2.52 g) solidified on standing. Purification by chromatography on silica gel, eluting with 0-100% EtOAc in heptanes, afforded the title compound (2.04 g, 79%) as a pale yellow oil which solidified on standing. δ_{H} (500 MHz, CDCl₃) 8.68 (d, J 2.0 Hz, 1H), 8.08 (dd, J 8.3, 2.2 Hz, 1H), 7.93 (d, J 8.3 Hz, 1H), 2.84 (s, 3H).

### Intermediate 6

### N-[(5-Bromopyridin-2-yl)(methyl)oxo-λ⁶-sulfanylidene]-2,2,2-trifluoroacetamide

Prepared from Intermediate 5 by a method analogous to that reported by Bolm et al., Organic Letters, 2004, 6(8), 1305-1307, using trifluoroacetamide and (diacetoxy-iodo)benzene in the presence of dirhodium tetraacetate. δ_{H} (500 MHz, CDCl₃) 8.79 (d, J 1.4 Hz, 1H), 8.22-8.19 (m, 1H), 8.18 (dd, J 8.4, 2.0 Hz, 1H), 3.56 (s, 3H).

### Intermediates 7 & 8

N-[(5-Bromopyridin-2-yl)(methyl)oxo-λ⁶-sulfanylidene]-2,2,2-trifluoroacetamide (Isomer A and Isomer B)

The title compounds may be prepared by chiral HPLC separation of Intermediate 6 to give Intermediate 7 (Isomer A) and Intermediate 8 (Isomer B).

### Intermediate 9

### 1-[5-(4,4,5,5-Tetramethyl-1,3,2-dioxaborolan-2-yl)pyrimidin-2-yl]-3-(trifluoromethyl)-azetidin-3-ol

2-Chloro-5-(4,4,5,5-tetramethyl-1,3,2-dioxaborolan-2-yl)pyrimidine (1 equiv.), 3-(trifluoromethyl)azetidin-3-ol (4 equiv.) and triethylamine (1 equiv.) were stirred in ethanol at 20° C. for 1 h. Water was slowly added to the reaction mixture. The resulting precipitate was filtered and washed with water to afford the title compound. δ_{H} (400 MHz, DMSO-d₆) 8.53 (s, 2H), 7.46 (s, 1H), 4.32 (d, J 10.8 Hz, 2H), 4.10 (d, J 10.8 Hz, 2H), 1.29 (s, 12H).

### Intermediate 10

### 2,2-Dichloro-3-oxocyclobutyl 2,2-dimethylpropanoate

To a stirred mixture of vinyl pivalate (30 g, 234 mmol) and zinc (31 g, 474 mmol) in diethyl ether (250 mL) was added a solution of 2,2,2-trichloroacetyl chloride (34 mL, 304 mmol) in diethyl ether (250 mL) dropwise over 2.5 h in a water bath whilst maintaining the reaction temperature at 15-30° C. The reaction mixture was filtered through Celite and washed through with ethyl acetate (200 mL). The filtrate was washed with water (200 mL) and brine (200 mL), then dried over sodium sulfate and concentrated under vacuum, to afford the title compound (68 g, 97% at 80% purity) as an orange liquid. δ_{H} (500 MHz, CDCl₃) 5.40 (dd, J 8.4, 6.2 Hz, 1H), 3.70 (dd, J 18.9, 8.4 Hz, 1H), 3.39 (dd, J 18.9, 6.2 Hz, 1H), 1.28 (s, 9H).

### Intermediate 11

### 3-Oxocyclobutyl 2,2-dimethylpropanoate

Zinc (74 g, 1.1 mol) was added to acetic acid (200 mL) with stirring and the suspension was cooled in an ice bath. Intermediate 10 (80%, 68 g, 228 mmol) in acetic acid (300 mL) was added dropwise over 2 h. The reaction mixture was warmed to room temperature and stirred for 1.5 h, then filtered and washed with DCM (100 mL). The filtrate was diluted with ethyl acetate (800 mL), then washed sequentially with water (3×250 mL), saturated aqueous NaHCO₃ solution (3×250 mL) and brine (50 mL). The organic phase was dried over sodium sulfate and concentrated under vacuum. The resulting brown oil (30 g) was purified by dry flash chromatography on silica gel, eluting with 0-10% ethyl acetate in heptanes, to afford the title compound (11 g, 28%) as a clear colourless oil. δ_{H} (500 MHz, CDCl₃) 5.26-5.19 (m, 1H), 3.51-3.40 (m, 2H), 3.19-3.07 (m, 2H), 1.22 (s, 9H).

### Intermediate 12

### 3-(5-Bromopyrimidin-2-yl)-3-hydroxycyclobutyl 2,2-dimethylpropanoate

5-Bromo-2-iodopyrimidine (16.7 g, 58.8 mmol) was dissolved in DCM (200 mL) with stirring and cooled to −78° C. under N₂. n-Butyllithium in hexane (2.5M, 23.5 mL) was added dropwise and the mixture was stirred for 20 minutes at −78° C. Intermediate 11 (10 g, 58.8 mmol) in DCM (50 mL) was cooled in a dry-ice bath and added in one portion. The reaction mixture was stirred at −78° C. for 10 minutes, then quenched by addition of saturated aqueous NH₄Cl solution (20 mL). The mixture was allowed to warm to room temperature, then saturated aqueous NH₄Cl solution (50 mL) was added and the mixture was extracted with DCM (2×100 mL). The combined organic extracts were dried over sodium sulfate and concentrated under vacuum. The crude residue was purified by column chromatography, using 0-30% ethyl acetate in heptane, to afford the title compound (7.6 g, 35%) as a yellow solid. δ_{H} (500 MHz, CDCl₃) 8.78 (s, 2H), 5.22-5.14 (m, 1H), 3.03-2.93 (m, 2H), 2.67-2.58 (m, 2H), 1.22 (s, 9H).

### Intermediate 13

### 1-(5-Bromopyrimidin-2-yl)cyclobutane-1,3-diol

Intermediate 12 (90%, 6 g, 16.4 mmol) was dissolved in MeOH (120 mL) and K₂CO₃ (11.3 g, 82 mmol) was added. The reaction mixture was stirred for 18 h at room temperature, then diluted with DCM (400 mL) and washed with water (150 mL). The aqueous phase was extracted with DCM (200 mL). The combined organic extracts were dried over sodium sulfate and concentrated under vacuum to afford the title compound (2.94 g, 73%) as an off-white solid. δ_{H} (500 MHz, DMSO-d₆) 8.98 (s, 2H), 5.63 (s, 1H), 5.08 (d, J 6.2 Hz, 1H), 4.09-3.92 (m, 1H), 2.87-2.79 (m, 2H), 2.28-2.14 (m, 2H).

### Intermediate 14

### 3-(5-Bromopyrimidin-2-yl)-3-hydroxycyclobutan-1-one

To a stirred solution of Intermediate 13 (2 g, 8.1 mmol) in DCM (200 mL) was added Dess-Martin periodinane (4.1 g, 9.8 mmol). The reaction mixture was stirred for 18 h, then the resulting suspension was diluted with DCM (100 mL) and washed with saturated aqueous NaHCO₃ solution (100 mL). The aqueous layer was re-extracted with DCM (100 mL), then the combined organic extracts were dried over sodium sulfate and concentrated. The crude residue was purified by chromatography on silica gel, eluting with 0-30% ethyl acetate in heptanes, to afford the title compound (1.37 g, 69%) as an off-white solid. δ_{H} (500 MHz, DMSO-d₆) 9.04 (s, 2H), 6.41 (s, 1H), 3.69-3.55 (m, 2H), 3.37-3.21 (m, 2H).

### Intermediate 15

### 3-(5-Bromopyrimidin-2-yl)-3-[(tert-butyldimethylsilyl)oxy]cyclobutan-1-one

Intermediate 14 (1.37 g, 5.64 mmol) was dissolved in dry DMF (20 mL) with stirring under N₂ and cooled to 0° C. 1H-Imidazole (1.9 g, 28.18 mmol) was added, followed by tert-butyl(chloro)dimethylsilane (2.0 g, 13.5 mmol). The reaction mixture was stirred at room temperature for 20 h, then diluted with DCM (150 mL) and washed with water (3×50 mL). The aqueous phase was re-extracted with DCM (50 mL). The combined organic extracts were dried over sodium sulfate and concentrated. The crude residue was purified by chromatography on silica gel, eluting with 0-20% ethyl acetate in heptanes, to afford the title compound (1.6 g, 79%) as a pale orange oil. δ_{H} (500 MHz, DMSO-d₆) 9.06 (s, 2H), 3.78-3.66 (m, 2H), 3.44-3.34 (m, 2H), 0.88 (s, 9H), 0.00 (s, 6H).

### Intermediate 16

### 3-(5-Bromopyrimidin-2-yl)-3-[(tert-butyldimethylsilyl)oxy]-1-methylcyclobutan-1-ol

Intermediate 15 (1.35 g, 3.78 mmol) was dissolved in dry diethyl ether (40 mL) under N₂ with stirring, then cooled to 0° C. using an ice bath. Methylmagnesium bromide in diethyl ether (3M, 2.52 mL) was added dropwise. The reaction mixture was stirred for 30 minutes at 0° C., then quenched with saturated aqueous NH₄Cl solution (20 mL) and water (20 mL). The mixture was extracted with ethyl acetate (2×50 mL), then dried over sodium sulfate and concentrated. The resulting yellow oil was purified by chromatography on silica gel, eluting with 0-100% DCM in heptane followed by 0-20% ethyl acetate in DCM, to afford the title compound (1.19 g, 84%), mixture of cis and trans isomers, as a clear oil.

Major isomer, approximately 70% abundance: δ_{H} (500 MHz, CDCl₃) 8.79 (s, 2H), 3.10-3.03 (m, 2H), 2.59-2.51 (m, 2H), 1.18 (s, 3H), 0.87 (s, 9H), −0.14 (s, 6H).

Minor isomer, approximately 30% abundance: δ_{H} (500 MHz, CDCl₃) 8.79 (s, 2H), 2.78-2.63 (m, 4H), 1.49 (s, 3H), 0.95 (s, 9H), 0.04 (s, 6H).

### Intermediate 17

### 3-[(tert-Butyldimethylsilyl)oxy]-1-methyl-3-[5-(4,4,5,5-tetramethyl-1,3,2-dioxaborolan-2-yl)pyrimidin-2-yl]cyclobutan-1-ol

The title compound may be prepared from Intermediate 16 and bis(pinacolato)-diboron in the presence of potassium acetate and a palladium catalyst by a method analogous to that reported by Ishiyama et al., Journal of Organic Chemistry, 1995, 60(23), 7508-7510. δ_{H} (500 MHz, CDCl₃) 9.02 (s, 2H), 3.15-3.08 (m, 2H), 2.58-2.50 (m, 2H), 1.37 (s, 12H), 1.27 (s, 3H), 0.87 (s, 9H), −0.16 (s, 6H).

### Intermediate 18

### (1s,3r)-3-(5-Bromopyrimidin-2-yl)-3-[(tert-butyldimethylsilyl)oxy]-1-ethylcyclobutan-1-ol

Prepared from Intermediate 15 and ethylmagnesium bromide by a method analogous to that used to prepare Intermediate 16. δ_{H} (500 MHz, CDCl₃) 8.78 (s, 2H), 3.08-3.02 (m, 2H), 2.48-2.43 (m, 2H), 1.38 (q, J 7.4 Hz, 2H), 0.87 (s, 9H), 0.84 (t, J 7.4 Hz, 3H), −0.14 (s, 6H).

### Intermediate 19

### (1s,3r)-3-[(tert-Butyldimethylsilyl)oxy]-1-ethyl-3-[5-(4,4,5,5-tetramethyl-1,3,2-dioxa-borolan-2-yl)pyrimidin-2-yl]cyclobutan-1-ol

Prepared from Intermediate 18 by a method analogous to that used to prepare Intermediate 17. δ_{H} (500 MHz, CDCl₃) 9.01 (s, 2H), 3.13-3.07 (m, 2H), 2.48-2.43 (m, 2H), 1.37 (s, 14H), 0.88 (s, 9H), 0.83 (t, J 7.4 Hz, 3H), −0.16 (s, 6H).

### Example 1

### (5-Bromo-2-methylindazol-3-yl)[2-(difluoromethoxy)phenyl]methanol

5-Bromo-2-methyl-2H-indazole (500 mg, 2.30 mmol) was dissolved in THF (15 mL) and cooled to 0° C. Lithium diisopropylamide solution (2.0M, 1.3 mL, 2.6 mmol) was added and the mixture was stirred for 30 minutes. 2-(Difluoromethoxy)benzaldehyde (444 mg, 2.53 mmol) was added and the mixture was stirred with warming to room temperature overnight. The reaction mixture was quenched with water (30 mL) and saturated aqueous sodium carbonate solution (30 mL), then extracted with ethyl acetate (100 mL). The organic layer was dried (sodium sulfate) and concentrated in vacuo. Purification by chromatography (silica, 25 g, 25-50% gradient of ethyl acetate in isohexanes) gave the title compound (280 mg, 32%) as a pale yellow solid. δ_{H} (DMSO-d₆) 7.88 (m, 1H), 7.52-7.36 (m, 3H), 7.24-7.14 (m, 3H), 7.09 (t, 1H, J 73.9 Hz, OCHF₂), 6.45 (d, 1H, J 4.7 Hz), 6.41 (d, 1H, J 4.7 Hz), 4.16 (s, 3H). LCMS (pH 10) MH⁺ 383.6/385.6, RT 2.22 minutes.

### Example 2

### 2-[5-(3-{[2-(Difluoromethoxy)phenyl](hydroxy)methyl}-2-methylindazol-5-yl)-pyrimidin-2-yl]propan-2-ol

Example 1 (260 mg, 0.68 mmol) and 2-(1-hydroxy-1-methylethyl)pyrimidine-5-boronic acid pinacol ester (233 mg, 0.88 mmol) were dissolved in 1,4-dioxane (10 mL). [1,1′-Bis(diphenylphosphino)ferrocene]dichloropalladium(II), complex with dichloro-methane (22.6 mg, 0.027 mmol) and 2M aqueous sodium carbonate solution (2 mL) were added. The mixture was degassed, refilled with nitrogen and heated at 100° C. for 6 h. The mixture was partitioned between ethyl acetate (50 mL) and water (50 mL), then the organic layer was dried (sodium sulfate) and concentrated in vacuo. The residue was purified by chromatography (silica, 10 g, 70-100% gradient of ethyl acetate in isohexanes) and concentrated in vacuo to give the title compound (180 mg, 60.2%) as a pale yellow solid. δ_{H} (DMSO-d₆) 9.23 (s, 2H), 8.01 (m, 1H), 7.68 (dd, 1H, J 9.0, 0.7 Hz), 7.57 (dd, 1H, J 9.0, 1.7 Hz), 7.46-7.42 (m, 2H), 7.33 (m, 1H), 7.18 (m, 1H), 7.09 (t, 1H, J 73.8 Hz, OCHF₂), 6.50 (m, 2H), 5.08 (s, 1H), 4.21 (s, 3H), 1.53 (s, 6H). LCMS (pH 10) MH⁺441.6, RT 1.82 minutes.

### Example 3

### 5-Bromo-3-{[2-(difluoromethoxy)phenyl]methyl}-2-methylindazole

Example 1 (1.00 g, 2.61 mmol) was dissolved in acetonitrile (30 mL). Sodium iodide (2.35 g, 15.7 mmol) was added and the mixture was stirred and heated to 60° C. Chlorotrimethylsilane (1.72 g, 15.7 mmol) was added. The mixture was stirred at 60° C. for 6 h, then quenched with saturated aqueous sodium carbonate solution (75 mL) and extracted with ethyl acetate (100 mL). The organic layer was washed twice with saturated aqueous sodium sulfite solution (75 mL then 25 mL), then dried (Na₂SO₄) and concentrated in vacuo. Purification by chromatography (silica, 25 g, 25-50% gradient of EtOAc in isohexanes) gave the title compound (550 mg, 57%) as a white solid. δ_{H} (DMSO-d₆) 7.69 (dd, 1H, J 1.8, 0.6 Hz), 7.52 (dd, 1H, J 9.1, 0.6 Hz), 7.37 (m, 1H), 7.35-7.13 (m, 4H), 7.26 (t, 1H, J_{H-F} 74.0 Hz), 4.46 (s, 2H), 4.06 (s, 3H). LCMS (pH 10) MH⁺ 367.6/369.6, RT 2.58 minutes.

### Example 4

### 2-[5-(3-{[2-(Difluoromethoxy)phenyl]methyl}-2-methylindazol-5-yl)pyrimidin-2-yl]-propan-2-ol

To a mixture of Example 3 (150 mg, 0.408 mmol), 2-(1-hydroxy-1-methylethyl)-pyrimidine-5-boronic acid pinacol ester (140 mg, 0.53 mmol) and [1,1′-bis(diphenyl-phosphino)ferrocene]dichloropalladium(II), complex with dichloromethane (13.6 mg, 0.0163 mmol) were added 1,4-dioxane (10 mL) and 2M aqueous sodium carbonate solution (2 mL). The mixture was degassed, then refilled with nitrogen and heated at 100° C. for 18 h. The mixture was cooled, diluted with ethyl acetate (100 mL) and washed with water (50 mL), then dried (Na₂SO₄) and concentrated in vacuo. The residue was purified by chromatography (silica, 10 g, 70-100% gradient of EtOAc in isohexanes) to give the title compound (162 mg, 93%) as a white solid. δ_{H} (DMSO-d₆) 9.05 (s, 2H), 7.92 (s, 1H), 7.69 (dd, 1H, J 9.0, 0.7 Hz), 7.63 (dd, 1H, J 9.0, 1.6 Hz), 7.33 (m, 1H), 7.30 (t, 1H, J_{H-F} 74.0 Hz), 7.24-7.19 (m, 3H), 5.09 (s, 1H), 4.55 (s, 2H), 4.09 (s, 3H), 1.54 (s, 6H). LCMS (pH 10) MH⁺ 425.8, RT 2.12 minutes.

### Example 5

### 1-[5-(3-{[2-(Difluoromethoxy)phenyl]methyl}-2-methylindazol-5-yl)pyrimidin-2-yl]-3-(trifluoromethyl)azetidin-3-ol

To a mixture of Example 3 (150 mg, 0.408 mmol), Intermediate 9 (197 mg, 0.57 mmol) and [1,1′-bis(diphenylphosphino)ferrocene]dichloropalladium(II), complex with dichloromethane (13.6 mg, 0.0163 mmol) were added 1,4-dioxane (10 mL) and 2M aqueous sodium carbonate solution (2 mL). The mixture was degassed, then refilled with nitrogen and heated at 100° C. for 18 h. The mixture was cooled, diluted with ethyl acetate (100 mL) and washed with water (50 mL), then dried (Na₂SO₄) and concentrated in vacuo. The residue was purified by chromatography (silica, 10 g, 70-100% gradient of EtOAc in isohexanes) to give the title compound (140 mg, 68%) as a white solid. δ_{H} (DMSO-d₆) 8.67 (s, 2H), 7.68 (dd, 1H, J 9.0, 0.6 Hz), 7.50 (dd, 1H, J 9.0, 1.6 Hz), 7.47-7.33 (m, 2H), 7.29 (t, 1H, J_{H-F} 74.0 Hz), 7.24-7.18 (m, 3H), 4.51 (s, 2H), 4.32 (m, 2H), 4.10 (m, 2H), 4.07 (s, 3H). LCMS (pH 10) MH⁺ 506.6, RT 2.30 minutes.

### Example 6

### 1-[5-(3-{[2-(Difluoromethoxy)phenyl](hydroxy)methyl}-2-methylindazol-5-yl)-pyrimidin-2-yl]-3-(trifluoromethyl)azetidin-3-ol

To a mixture of Example 1 (500 mg, 1.30 mmol), Intermediate 9 (630 mg, 1.83 mmol) and [1,1′-bis(diphenylphosphino)ferrocene]dichloropalladium(II), complex with dichloromethane (43.5 mg, 0.0522 mmol) were added 1,4-dioxane (20 mL) and 2M aqueous sodium carbonate solution (4 mL). The mixture was degassed, then refilled with nitrogen and heated at 100° C. overnight. The mixture was cooled, diluted with ethyl acetate (150 mL) and washed with water (100 mL), then dried (Na₂SO₄) and concentrated in vacuo. The residue was crystallised from DCM/isohexanes to give the title compound (540 mg, 79.4%) as an off-white solid. δ_{H} (DMSO-d₆) 8.50 (s, 2H), 7.99 (dd, 1H, J 7.0, 2.4 Hz), 7.61 (dd, 1H, J 9.0, 0.6 Hz), 7.47-7.41 (m, 4H), 7.16 (m, 1H), 7.09 (t, 1H, J_{H-F} 73.8 Hz), 7.07 (m, 1H), 6.48-6.42 (m, 2H), 4.33-4.30 (m, 2H), 4.21 (s, 3H), 4.14-4.11 (m, 2H). LCMS (pH 10) MH⁺ 522.6, RT 2.06 minutes.

### Example 7

### (2R)-8-Fluoro-4-({5-[2-(2-hydroxypropan-2-yl)pyrimidin-5-yl]-2-methyl-2H-indazol-3-yl}methyl)-2-methyl-3,4-dihydro-2H-1,4-benzoxazin-3-one

2-[5-(4,4,5,5-Tetramethyl-1,3,2-dioxaborolan-2-yl)pyrimidin-2-yl]propan-2-ol (110 mg, 0.42 mmol) and Intermediate 4 (165 mg, 0.41 mmol) were dissolved in 1,4-dioxane (6 mL) and 2M aqueous potassium carbonate solution (0.6 mL) was added. The mixture was degassed with nitrogen for 5 minutes, then Pd(dppf)Cl₂ complex with DCM (17 mg, 0.021 mmol) was added. The mixture was stirred at 100° C. under nitrogen for 2 h. The dark reaction mixture was allowed to cool, then diluted with ethyl acetate (10 mL). The resulting mixture was filtered through celite and washed through with ethyl acetate (20 mL). The filtrate was dried over MgSO₄ and concentrated in vacuo. The resulting dark oil was purified by chromatography on silica gel, eluting with a gradient of 0-100% ethyl acetate in heptane. The purified residue was dissolved in 1:1 acetonitrile/water and freeze-dried to give the title compound (94 mg, 48.1%) as a pale beige-coloured solid. δ_{H} (500 MHz, CDCl₃) 8.87 (s, 2H), 7.79 (dd, J 8.9, 0.7 Hz, 1H), 7.68-7.65 (m, 1H), 7.46 (dd, J 9.0, 1.6 Hz, 1H), 6.90-6.84 (m, 2H), 6.84-6.80 (m, 1H), 5.65 (d, J 16.4 Hz, 1H), 5.55 (d, J 16.4 Hz, 1H), 4.79 (q, J 6.8 Hz, 1H), 4.68 (s, 1H), 4.24 (s, 3H), 1.67 (d, J 6.8 Hz, 3H), 1.66 (s, 6H). Method D HPLC-MS: MH⁺ m/z 462, RT 3.04 minutes.

### Example 8

### (2R)-8-Fluoro-4-[(5-{6-[imino(methyl)oxo-λ⁶-sulfanyl]pyridin-3-yl}-2-methyl-2H-indazol-3-yl)methyl]-2-methyl-3,4-dihydro-2H-1,4-benzoxazin-3-one

A mixture of Intermediate 6 (80 mg, 0.24 mmol), bis(pinacolato)diboron (65 mg, 0.26 mmol) and potassium acetate (65 mg, 0.66 mmol) in anhydrous 1,4-dioxane (3 mL) was degassed with nitrogen for 5 minutes. Pd(dppf)Cl₂ complex with DCM (10 mg, 0.01 mmol) was added. The mixture was stirred at 80° C. under nitrogen for 2 h in a sealed tube, then allowed to cool to room temperature. Intermediate 4 (80%, 110 mg, 0.22 mmol) was added, followed by 2M aqueous potassium carbonate solution (0.32 mL) and additional Pd(dppf)Cl₂ complex with DCM (10 mg, 0.01 mmol). The mixture was stirred at 100° C. for 3 h, then allowed to cool to ambient temperature and diluted with ethyl acetate (10 mL). The mixture was filtered through a pad of Celite, washing through with ethyl acetate (10 mL). The filtrate was concentrated under vacuum. The residue was purified by chromatography on silica gel, eluting with a gradient of 0-100% ethyl acetate in heptane followed by a gradient of 0-15% methanol in ethyl acetate. Further purification by preparative HPLC, followed by freeze-drying from a mixture of acetonitrile and water, afforded the title compound (57 mg, 53.5%) as an off-white solid. δ_{H} (500 MHz, CDCl₃) 8.90-8.84 (m, 1H), 8.18 (d, J 8.1 Hz, 1H), 8.01 (dd, J 8.1, 2.2 Hz, 1H), 7.78 (d, J 9.0 Hz, 1H), 7.68 (s, 1H), 7.49 (dd, J 9.0, 1.7 Hz, 1H), 6.91-6.84 (m, 2H), 6.84-6.80 (m, 1H), 5.66 (dd, J 16.4, 1.6 Hz, 1H), 5.55 (d, J 16.4 Hz, 1H), 4.78 (q, J 6.8 Hz, 1H), 4.26 (s, 3H), 3.31 (s, 3H), 1.66 (d, J 6.8 Hz, 3H). Method D HPLC-MS: MH⁺ m/z 480.1, RT 2.40 minutes.

### Example 9

### (2R)-8-Fluoro-4-[(5-{6-[imino(methyl)oxo-λ⁶-sulfanyl]pyridin-3-yl}-2-methyl-2H-indazol-3-yl)methyl]-2-methyl-3,4-dihydro-2H-1,4-benzoxazin-3-one (Isomer A)

Prepared from Intermediate 4 and Intermediate 7 by a method analogous to that described for Example 8. δ_{H} (500 MHz, CD₃OD) 8.91-8.83 (m, 1H), 8.22-8.14 (m, 2H), 7.82 (s, 1H), 7.69 (d, J 9.0 Hz, 1H), 7.62 (dd, J 9.0, 1.5 Hz, 1H), 7.14 (d, J 8.3 Hz, 1H), 6.98 (td, J 8.3, 5.5 Hz, 1H), 6.91 (t, J 8.7 Hz, 1H), 5.83 (d, J 16.7 Hz, 1H), 5.68 (d, J 16.7 Hz, 1H), 4.85-4.81 (m, 1H), 4.27 (s, 3H), 3.30 (s, 3H), 1.60 (d, J 6.7 Hz, 3H). Method D HPLC-MS: MH⁺ m/z 480, RT 2.41 minutes.

### Example 10

### (2R)-8-Fluoro-4-[(5-{6-[imino(methyl)oxo-λ⁶-sulfanyl]pyridin-3-yl}-2-methyl-2H-indazol-3-yl)methyl]-2-methyl-3,4-dihydro-2H-1,4-benzoxazin-3-one (Isomer B)

Prepared from Intermediate 4 and Intermediate 8 by a method analogous to that described for Example 8. δ_{H} (500 MHz, CD₃OD) 8.87 (dd, J 2.0, 0.9 Hz, 1H), 8.23-8.11 (m, 2H), 7.82 (dd, J 1.6, 0.9 Hz, 1H), 7.69 (dd, J 9.0, 0.8 Hz, 1H), 7.63 (dd, J 9.0, 1.7 Hz, 1H), 7.14 (d, J 8.3 Hz, 1H), 6.99 (td, J 8.4, 5.5 Hz, 1H), 6.91 (ddd, J 9.8, 8.5, 1.3 Hz, 1H), 5.83 (d, J 16.7 Hz, 1H), 5.69 (d, J 16.7 Hz, 1H), 4.62-4.47 (m, 1H), 4.27 (s, 3H), 3.30 (s, 3H), 1.60 (d, J 6.8 Hz, 3H). Method D HPLC-MS: MH⁺ m/z 480, RT 2.41 minutes.

### Example 11

### (2R)-8-Fluoro-4-[(5-{2-[3-hydroxy-3-(trifluoromethyl)azetidin-1-yl]pyrimidin-5-yl}-2-methyl-2H-indazol-3-yl)methyl]-2-methyl-3,4-dihydro-2H-1,4-benzoxazin-3-one

Prepared from Intermediate 4 and Intermediate 9 by a method analogous to that described for Example 7. δ_{H} (500 MHz, DMSO-d₆) 8.61 (s, 2H), 7.67 (s, 1H), 7.62 (d, J 9.0 Hz, 1H), 7.48 (dd, J 9.0, 1.6 Hz, 1H), 7.43 (s, 1H), 7.22-7.16 (m, 1H), 7.02-6.95 (m, 2H), 5.79 (d, J 16.7 Hz, 1H), 5.65 (d, J 16.7 Hz, 1H), 5.01 (q, J 6.7 Hz, 1H), 4.33 (d, J 10.5 Hz, 2H), 4.17 (s, 3H), 4.11 (d, J 10.0 Hz, 2H), 1.54 (d, J 6.7 Hz, 3H). Method D HPLC-MS: MH⁺ m/z 543, RT 3.12 minutes.

### Example 12

### (2R)-8-Fluoro-2-methyl-4-[(2-methyl-5-{2-[(1r,3s)-1,3-dihydroxy-3-methylcyclobutyl]-pyrimidin-5-yl}-2H-indazol-3-yl)methyl]-3,4-dihydro-2H-1,4-benzoxazin-3-one

Prepared from Intermediate 4 and Intermediate 17 by a method analogous to that described for Example 7, followed by treatment with triethylamine trihydrofluoride in 2-methyltetrahydrofuran at 60° C. δ_{H} (500 MHz, CDCl₃) 8.87 (s, 2H), 7.79 (dd, J 9.0, 0.7 Hz, 1H), 7.65 (s, 1H), 7.46 (dd, J 8.9, 1.6 Hz, 1H), 6.90-6.83 (m, 2H), 6.83-6.79 (m, 1H), 5.66 (d, J 16.4 Hz, 1H), 5.55 (d, J 16.4 Hz, 1H), 4.94 (s, 1H), 4.78 (d, J 6.8 Hz, 1H), 4.25 (s, 3H), 3.02-2.96 (m, 2H), 2.60 (s, 1H), 2.53-2.45 (m, 2H), 1.67 (d, J 6.8 Hz, 3H), 1.59 (s, 3H). Method D HPLC-MS: MH⁺ m/z 504, RT 2.49 minutes.

### Example 13

### (2R)-8-Fluoro-2-methyl-4-[(2-methyl-5-{2-[(1r,3s)-3-ethyl-1,3-dihydroxycyclobutyl]-pyrimidin-5-yl}-2H-indazol-3-yl)methyl]-3,4-dihydro-2H-1,4-benzoxazin-3-one

Prepared from Intermediate 4 and Intermediate 19 by a method analogous to that described for Example 7, followed by treatment with TBAF. δ_{H} (500 MHz, CDCl₃) 8.87 (s, 2H), 7.79 (d, J 9.0 Hz, 1H), 7.65 (s, 1H), 7.45 (dd, J 9.0, 1.5 Hz, 1H), 6.90-6.84 (m, 2H), 6.81 (dd, J 5.8, 2.8 Hz, 1H), 5.66 (d, J 16.4 Hz, 1H), 5.55 (d, J 16.4 Hz, 1H), 4.96 (s, 1H), 4.78 (q, J 6.8 Hz, 1H), 4.25 (s, 3H), 2.96 (d, J 14.0 Hz, 2H), 2.40 (d, J 14.0 Hz, 2H), 1.87 (q, J 7.4 Hz, 2H), 1.66 (d, J 6.8 Hz, 3H), 1.00 (t, J 7.4 Hz, 3H). Method D HPLC-MS: MH⁺ m/z 518, RT 2.73 minutes.

1. A compound of formula (I) or an N-oxide thereof, or a pharmaceutically acceptable salt thereof: 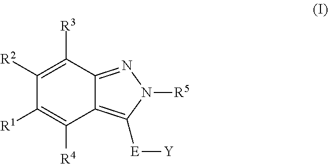
wherein
E represents a covalent bond; or E represents —O—, —S—, —S(O)—, —S(O)₂— or —N(R⁶)—; or E represents an optionally substituted straight or branched C₁₋₄ alkylene chain;
Y represents Y¹ or Y²;
Y¹ represents C₃₋₇ cycloalkyl, aryl, C₃₋₇ heterocycloalkyl or heteroaryl, any of which groups may be optionally substituted by one or more substituents;
Y² represents a group of formula (Ya), (Yb), (Yc), (Yd), (Ye) or (Yf): 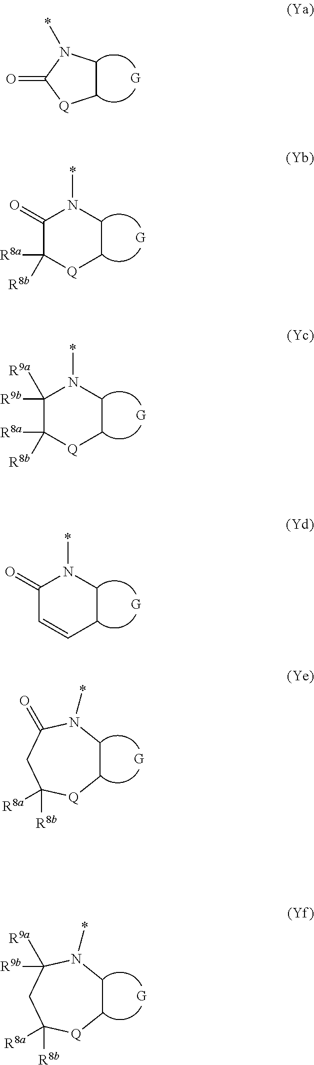
the asterisk (*) represents the point of attachment to the remainder of the molecule;
Q represents —O—, —S—, —S(O)—, —S(O)₂—, —S(O)(NR⁶)—, —N(R⁶)—, —C(O)— or —C(R^{7a})(R^{7b})—;
G represents the residue of an optionally substituted benzene ring; or an optionally substituted five-membered heteroaromatic ring selected from furyl, thienyl, pyrrolyl, pyrazolyl, oxazolyl, isoxazolyl, thiazolyl, isothiazolyl, imidazolyl, oxadiazolyl, thiadiazolyl and triazolyl; or an optionally substituted six-membered heteroaromatic ring selected from pyridinyl, pyridazinyl, pyrimidinyl, pyrazinyl and triazinyl;
R¹, R², R³ and R⁴ independently represent hydrogen, halogen, cyano, nitro, hydroxy, trifluoromethyl, trifluoromethoxy, —OR^{a}, —SR^{a}, —SOR^{a}, —SO₂R^{a}, —SF₅, —NR^{b}R^{c}, —NR^{c}COR^{d}, —NR^{c}CO₂R^{d}, —NHCONR^{b}R^{c}, —NR^{c}SO₂R^{e}, —N(SO₂R^{e})₂, —NHSO₂NR^{b}R^{c}, —COR^{d}, —CO₂R^{d}, —CONR^{b}R^{c}, —CON(OR^{a})R^{b}, —SO₂NR^{b}R^{c} or —SO(NR^{b})R^{d}; or C₁₋₆ alkyl, C₂₋₆ alkenyl, C₂₋₆ alkynyl, C₃₋₇ cycloalkyl, C₄₋₇ cycloalkenyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, C₃₋₇ heterocycloalkenyl, C₄₋₉ heterobicycloalkyl, heteroaryl, heteroaryl(C₁₋₆)alkyl, (C₃₋₇)heterocycloalkyl(C₁₋₆)alkyl-aryl-, heteroaryl(C₃₋₇)heterocycloalkyl-, (C₃₋₇)cycloalkyl-heteroaryl-, (C₃₋₇)cycloalkyl(C₁₋₆)alkyl-heteroaryl-, (C₄₋₇)cycloalkenyl-heteroaryl-, (C₄₋₉)bicycloalkyl-heteroaryl-, (C₃₋₇)heterocycloalkyl-heteroaryl-, (C₃₋₇)heterocycloalkyl(C₁₋₆)alkyl-heteroaryl-, (C₃₋₇)heterocycloalkenyl-heteroaryl-, (C₄₋₉)heterobicycloalkyl-heteroaryl- or (C₄₋₉)spiroheterocycloalkyl-heteroaryl-, any of which groups may be optionally substituted by one or more substituents;
R⁵ represents C₁₋₆ alkyl, optionally substituted by fluoro, hydroxy, C₁₋₆ alkoxy, amino, C₁₋₆ alkylamino or di(C₁₋₆)alkylamino;
R⁶ represents hydrogen or C₁₋₆ alkyl;
R^{7a} and R^{7b} independently represent hydrogen or C₁₋₆ alkyl;
R^{8a} and R^{8b} independently represent hydrogen, halogen or C₁₋₆ alkyl; or
R^{8a} and R^{8b}, when taken together with the carbon atom to which they are both attached, represent C₃₋₇ cycloalkyl or C₃₋₇ heterocycloalkyl, either of which groups may be optionally substituted by one or more substituents; or
R^{7a} and R^{8a}, when taken together with the two intervening carbon atoms, represent C₃₋₇ cycloalkyl or C₃₋₇ heterocycloalkyl, either of which groups may be optionally substituted by one or more substituents;
R^{9a} and R^{9b} independently represent hydrogen or C₁₋₆ alkyl; or
R^{9a} and R^{9b}, when taken together with the carbon atom to which they are both attached, represent C₃₋₇ cycloalkyl or C₃₋₇ heterocycloalkyl, either of which groups may be optionally substituted by one or more substituents;
R^{a} represents C₁₋₆ alkyl, aryl, aryl(C₁₋₆)alkyl, heteroaryl or heteroaryl(C₁₋₆)alkyl, any of which groups may be optionally substituted by one or more substituents;
R^{b} and R^{c} independently represent hydrogen or trifluoromethyl; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents; or
R^{b} and R^{c}, when taken together with the nitrogen atom to which they are both attached, represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents;
R^{d} represents hydrogen; or C₁₋₆ alkyl, C₃₋₇ cycloalkyl, aryl, C₃₋₇ heterocycloalkyl or heteroaryl, any of which groups may be optionally substituted by one or more substituents; and
R^{e} represents C₁₋₆ alkyl, aryl or heteroaryl, any of which groups may be optionally substituted by one or more substituents.

2. The compound as claimed in claim 1 represented by formula (IIA-1) or (IIA-2) or an N-oxide thereof, or a pharmaceutically acceptable salt thereof: 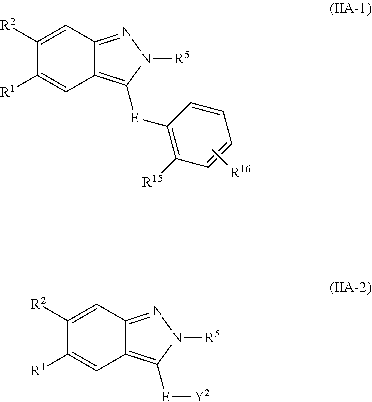
wherein
R¹⁵ and R¹⁶ independently represent hydrogen, halogen, cyano, nitro, C₁₋₆ alkyl, trifluoromethyl, hydroxy, C₁₋₆ alkoxy, difluoromethoxy, trifluoromethoxy, C₁₋₆ alkylthio, C₁₋₆ alkylsulfinyl, C₁₋₆ alkylsulfonyl, amino, C₁₋₆ alkylamino, di(C₁₋₆)alkylamino, arylamino, C₂₋₆ alkylcarbonylamino, C₁₋₆ alkylsulfonylamino, formyl, C₂₋₆ alkylcarbonyl, C₃₋₆ cycloalkylcarbonyl, C₃₋₆ heterocycloalkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl or di(C₁₋₆)alkylaminosulfonyl.

3. The compound as claimed in claim 2 represented by formula (IIB-1) or an N-oxide thereof, or a pharmaceutically acceptable salt thereof: 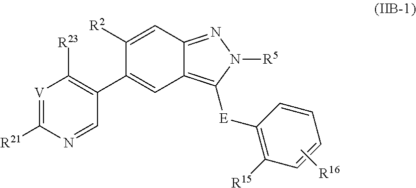
wherein
V represents C—R²² or N;
E represents a covalent bond; or E represents —O—, —S—, —S(O)—, —S(O)₂— or —N(R⁶)—; or E represents an optionally substituted straight or branched C₁₋₄ alkylene chain;
R² represents hydrogen, halogen, cyano, nitro, hydroxy, trifluoromethyl, trifluoromethoxy, —OR^{a}, —SR^{a}, —SOR^{a}, —SO₂R^{a}, —SF₅, —NR^{b}R^{c}, —NR^{c}COR^{d}, —NR^{c}CO₂R^{d}, —NHCONR^{b}R^{c}, —NR^{c}SO₂R^{e}, —N(SO₂R^{e})₂, —NHSO₂NR^{b}R^{c}, —COR^{d}, —CO₂R^{d}, —CONR^{b}R^{c}, —CON(OR^{a})R^{b}, —SO₂NR^{b}R^{c} or —SO(NR^{b})R^{d}; or C₁₋₆ alkyl, C₂₋₆ alkenyl, C₂₋₆ alkynyl, C₃₋₇ cycloalkyl, C₄₋₇ cycloalkenyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, C₃₋₇ heterocycloalkenyl, C₄₋₉ heterobicycloalkyl, heteroaryl, heteroaryl(C₁₋₆)alkyl, (C₃₋₇)heterocycloalkyl(C₁₋₆)alkyl-aryl-, heteroaryl(C₃₋₇)heterocycloalkyl-(C₃₋₇)cycloalkyl-heteroaryl-, (C₃₋₇)cycloalkyl(C₁₋₆)alkyl-heteroaryl-, (C₄₋₇)cycloalkenyl-heteroaryl-, (C₄₋₉)bicycloalkyl-heteroaryl-, (C₃₋₇)heterocycloalkyl-heteroaryl-, (C₃₋₇)heterocycloalkyl(C₁₋₆)alkyl-heteroaryl-, (C₃₋₇)heterocycloalkenyl-heteroaryl-, (C₄₋₉)heterobicycloalkyl-heteroaryl- or (C₄₋₉)spiroheterocycloalkyl-heteroaryl-, any of which groups may be optionally substituted by one or more substituents;
R⁵ represents C₁₋₆ alkyl, optionally substituted by fluoro, hydroxy, C₁₋₆ alkoxy, amino, C₁₋₆ alkylamino or di(C₁₋₆)alkylamino;
R²¹ represents hydrogen, halogen, halo(C₁₋₆)alkyl, cyano, C₁₋₆ alkyl, trifluoro-methyl, C₂₋₆ alkenyl, C₂₋₆ alkynyl, hydroxy, hydroxy(C₁₋₆)alkyl, C₁₋₆ alkoxy, (C₁₋₆)alkoxy-(C₁₋₆)alkyl, difluoromethoxy, trifluoromethoxy, trifluoroethoxy, carboxy(C₃₋₇)cycloalkyl-oxy, C₁₋₆ alkylthio, C₁₋₆ alkylsulphonyl, (C₁₋₆)alkylsulphonyl(C₁₋₆)alkyl, amino, amino-(C₁₋₆)alkyl, C₁₋₆ alkylamino, di(C₁₋₆)alkylamino, (C₁₋₆)alkoxy(C₁₋₆)alkylamino, N—[(C₁₋₆)-alkyl]-N-[hydroxy(C₁₋₆)alkyl]amino, C₂₋₆ alkylcarbonylamino, (C₂₋₆)alkylcarbonylamino-(C₁₋₆)alkyl, C₂₋₆ alkoxycarbonylamino, N—[(C₁₋₆)alkyl]-N-[carboxy(C₁₋₆)alkyl]amino, carboxy(C₃₋₇)cycloalkylamino, carboxy(C₃₋₇)cycloalkyl(C₁₋₆)alkylamino, C₁₋₆ alkyl-sulphonylamino, C₁₋₆ alkylsulphonylamino(C₁₋₆)alkyl, formyl, C₂₋₆ alkylcarbonyl, (C₂₋₆)alkylcarbonyloxy(C₁₋₆)alkyl, carboxy, carboxy(C₁₋₆)alkyl, C₂₋₆ alkoxycarbonyl, morpholinyl(C₁₋₆)alkoxycarbonyl, C₂₋₆ alkoxycarbonyl(C₁₋₆)alkyl, C₂₋₆ alkoxycarbonyl-methylidenyl, aminocarbonyl, C₁₋₆ alkylaminocarbonyl, di(C₁₋₆)alkylaminocarbonyl, aminosulphonyl, C₁₋₆ alkylaminosulphonyl, di(C₁₋₆)alkylaminosulphonyl, (C₁₋₆)alkyl-sulphoximinyl or [(C₁₋₆)alkyl][N—(C₁₋₆)alkyl]sulphoximinyl; or R²¹ represents (C₃₋₇)cycloalkyl, (C₃₋₇)cycloalkyl(C₁₋₆)alkyl, (C₄₋₇)cycloalkenyl, (C₄₋₉)bicycloalkyl, (C₃₋₇)heterocycloalkyl, (C₃₋₇)heterocycloalkenyl, (C₄₋₉)heterobicycloalkyl or (C₄₋₉)spiroheterocycloalkyl, any of which groups may be optionally substituted by one or more substituents;
R²² represents hydrogen, halogen or C₁₋₆ alkyl;
R²³ represents hydrogen, C₁₋₆ alkyl, trifluoromethyl or C₁₋₆ alkoxy.

4. The compound as claimed in claim 2 represented by formula (IIB-2) or an N-oxide thereof, or a pharmaceutically acceptable salt thereof: 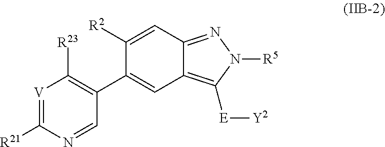
wherein
V represents C—R²² or N;
E represents a covalent bond: or E represents —O—, —S—, —S(O)—, —S(O)₂— or —N(R⁶)—; or E represents an optionally substituted straight or branched C₁₋₄ alkylene chain;
Y² represents a group of formula (Ya), (Yb), (Yc), (Yd), (Ye) or (Yf): 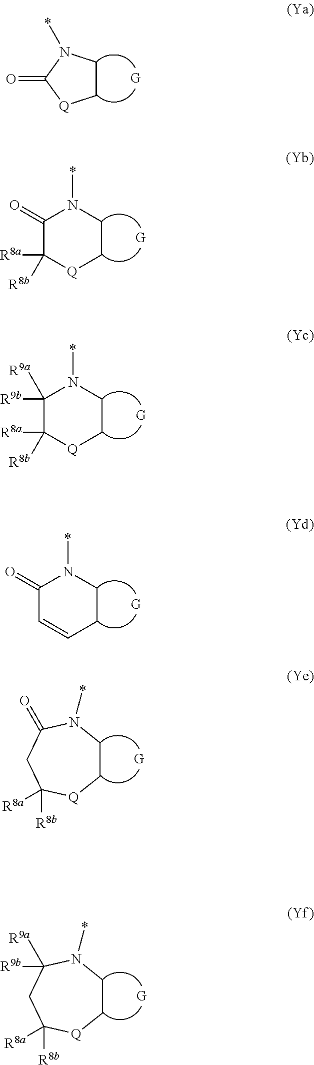
the asterisk (*) represents the point of attachment to the remainder of the molecule;
R² represents hydrogen, halogen, cyano, nitro, hydroxy, trifluoromethyl, trifluoromethoxy, —OR^{a}, —SR^{a}, —SOR^{a}, —SO₂R^{a}, —SF₅, —NR^{b}R^{c}, —NR^{c}COR^{d}, —NR^{c}CO₂R^{d}, —NHCONR^{b}R^{c}, —NR^{c}SO₂R^{e}, —N(SO₂R^{e})₂, —NHSO₂NR^{b}R^{c}, —COR^{d}, —CO₂R^{d}, —CONR^{b}R^{c}, —CON(OR^{a})R^{b}, —SO₂NR^{b}R^{c} or —SO(NR^{b})R^{d}; or C₁₋₆ alkyl, C₂₋₆ alkenyl, C₂₋₆ alkynyl, C₃₋₇ cycloalkyl, C₄₋₇ cycloalkenyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, C₃₋₇ heterocycloalkenyl, C₄₋₉ heterobicycloalkyl, heteroaryl, heteroaryl(C₁₋₆)alkyl, (C₃₋₇)heterocycloalkyl(C₁₋₆)alkyl-aryl-, heteroaryl(C₃₋₇)heterocycloalkyl, (C₃₋₇)cycloalkyl-heteroaryl-, (C₃₋₇)cycloalkyl(C₁₋₆)alkyl-heteroaryl-, (C₄₋₇)cycloalkenyl-heteroaryl-, (C₄₋₉)bicycloalkyl-heteroaryl-, (C₃₋₇)heterocycloalkyl-heteroaryl-, (C₃₋₇)heterocycloalkyl(C₁₋₆)alkyl-heteroaryl-, (C₃₋₇)heterocycloalkenyl-heteroaryl-, (C₄₋₉)heterobicycloalkyl-heteroaryl- or (C₄₋₉)spiroheterocycloalkyl-heteroaryl-any of which groups may be optionally substituted by one or more substituents;
R⁵ represents C₁₋₆ alkyl, optionally substituted by fluoro, hydroxy, C₁₋₆ alkoxy, amino, C₁₋₆ alkylamino or di(C₁₋₆)alkylamino;
R²¹ represents hydrogen, halogen, halo(C₁₋₆)alkyl, cyano, C₁₋₆ alkyl, trifluoro-methyl, C₂₋₆ alkenyl, C₂₋₆ alkynyl, hydroxy, hydroxy(C₁₋₆)alkyl, C₁₋₆ alkoxy, (C₁₋₆)alkoxy-(C₁₋₆)alkyl, difluoromethoxy, trifluoromethoxy, trifluoroethoxy, carboxy(C₃₋₇)cycloalkyl-oxy, C₁₋₆ alkylthio, C₁₋₆ alkylsulphonyl, (C₁₋₆)alkylsulphonyl(C₁₋₆)alkyl, amino, amino-(C₁₋₆)alkyl, C₁₋₆ alkylamino, di(C₁₋₆)alkylamino, (C₁₋₆)alkoxy(C₁₋₆)alkylamino, N—[(C₁₋₆)-alkyl]-N-[hydroxy(C₁₋₆)alkyl]amino, C₂₋₆ alkylcarbonylamino, (C₂₋₆)alkylcarbonylamino-(C₁₋₆)alkyl, C₂₋₆ alkoxycarbonylamino, N—[(C₁₋₆)alkyl]-N-[carboxy(C₁₋₆)alkyl]amino, carboxy(C₃₋₇)cycloalkylamino, carboxy(C₃₋₇)cycloalkyl(C₁₋₆)alkylamino, C₁₋₆ alkyl-sulphonylamino, C₁₋₆ alkylsulphonylamino(C₁₋₆)alkyl, formyl, C₂₋₆ alkylcarbonyl, (C₂₋₆)alkylcarbonyloxy(C₁₋₆)alkyl, carboxy, carboxy(C₁₋₆)alkyl, C₂₋₆ alkoxycarbonyl, morpholinyl(C₁₋₆)alkoxycarbonyl, C₂₋₆ alkoxycarbonyl(C₁₋₆)alkyl, C₂₋₆ alkoxycarbonyl-methylidenyl, aminocarbonyl, C₁₋₆ alkylaminocarbonyl, di(C₁₋₆)alkylaminocarbonyl, aminosulphonyl, C₁₋₆ alkylaminosulphonyl, di(C₁₋₆)alkylaminosulphonyl, (C₁₋₆)alkyl-sulphoximinyl or [(C₁₋₆)alkyl][N—(C₁₋₆)alkyl]sulphoximinyl; or R²¹ represents (C₃₋₇)cycloalkyl, (C₃₋₇)cycloalkyl(C₁₋₆)alkyl, (C₄₋₇)cycloalkenyl, (C₄₋₉)bicycloalkyl, (C₃₋₇)heterocycloalkyl, —(C₃₋₇)heterocycloalkenyl, (C₄₋₉)heterobicycloalkyl or (C₄₋₉)spiroheterocycloalkyl, n of which groups may be optionally substituted by one or more substituents;
R²³ represents hydrogen, C₁₋₆ alkyl, trifluoromethyl or C₁₋₆ alkoxy.

5. The compound as claimed in claim 3 wherein R²¹ represents hydroxy(C₁₋₆)alkyl.

6. The compound as claimed in claim 5 wherein R²¹ represents 2-hydroxyprop-2-yl.

7. The compound as claimed in claim 3 represented by formula (IIC-1) or (IID-1) or an N-oxide thereof, or a pharmaceutically acceptable salt thereof: 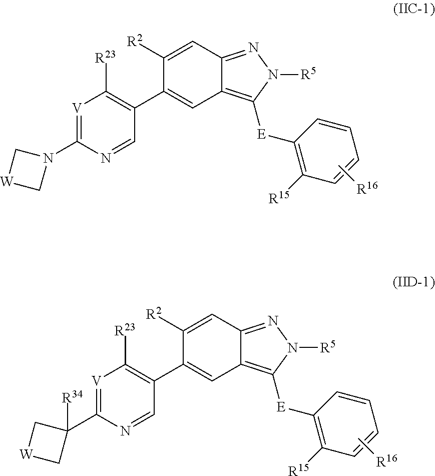
wherein
E represents a covalent bond; or E represents —O—, —S—, —S(O)—, —S(O)₂— or —N(R⁶)—; or E represents an optionally substituted straight or branched C₁₋₄ alkylene chain;
W represents O, S, S(O), S(O)₂, S(O)(NR⁶), N(R³¹) or C(R³²)(R³³);
R² represents hydrogen, halogen, cyano, nitro, hydroxy, trifluoromethyl, trifluoromethoxy, —OR^{a}, —SR^{a}, —SOR^{a}, —SO₂R^{a}, —SF₅, —NR^{b}R^{c}, —NR^{c}COR^{d}, —NR^{c}CO₂R^{d}, —NHCONR^{b}R^{c}, —NR^{c}SO₂R^{e}, —N(SO₂R^{e})₂, —NHSO₂NR^{b}R^{c}, —COR^{d}, —CO₂R^{d}, —CONR^{b}R^{c}, —CON(OR^{a})R^{b}, —SO₂NR^{b}R^{c} or —SO(NR^{b})R^{d}; or C₁₋₆ alkyl, C₂₋₆ alkenyl, C₂₋₆ alkynyl, C₃₋₇ cycloalkyl, C₄₋₇ cycloalkenyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, C₃₋₇ heterocycloalkenyl, C₄₋₉ heterobicycloalkyl, heteroaryl, heteroaryl(C₁₋₆)alkyl (C₃₋₇)heterocycloalkyl(C₁₋₆)alkyl-aryl-, heteroaryl(C₃₋₇)heterocycloalkyl-, (C₃₋₇)cycloalkyl-heteroaryl-, (C₃₋₇)cycloalkyl(C₁₋₆)alkyl-heteroaryl-, (C₄₋₇)cycloalkenyl-heteroaryl-, (C₄₋₉)bicycloalkyl-heteroaryl-, (C₃₋₇)heterocycloalkyl-heteroaryl-, (C₃₋₇)heterocycloalkyl(C₁₋₆)alkyl-heteroaryl-, (C₃₋₇)heterocycloalkenyl-heteroaryl-, (C₄₋₉)heterobicycloalkyl-heteroaryl- or (C₄₋₉)spiroheterocycloalkyl-heteroaryl-, any of which groups may be optionally substituted by one or more substituents;
R⁵ represents C₁₋₆ alkyl, optionally substituted by fluoro, hydroxy, C₁₋₆ alkoxy, amino, C₁₋₆ alkylamino or di(C₁₋₆)alkylamino;
R⁶ represents hydrogen or C₁₋₆ alkyl;
R¹⁵ and R¹⁶ independently represent hydrogen, halogen, cyano, nitro, C₁₋₆ alkyl, trifluoromethyl, hydroxy, C₁₋₆ alkoxy, difluoromethoxy, trifluoromethoxy, C₁₋₆ alkylthio, C₁₋₆ alkylsulfinyl, C₁₋₆ alkylsulfonyl, amino, C₁₋₆ alkylamino, di(C₁₋₆)alkylamino, arylamino, C₂₋₆ alkylcarbonylamino, C₁₋₆ alkylsulfonylamino, formyl, C₂₋₆ alkylcarbonyl, C₃₋₆ cycloalkylcarbonyl, C₃₋₆ heterocycloalkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl or di(C₁₋₆)alkylaminosulfonyl;
R³¹ represents hydrogen, cyano(C₁₋₆)alkyl, C₁₋₆ alkyl, trifluoromethyl, trifluoro-ethyl, C₁₋₆ alkylsulphonyl, (C₁₋₆)alkylsulphonyl(C₁₋₆)alkyl, formyl, C₂₋₆ alkylcarbonyl, carboxy, carboxy(C₁₋₆)alkyl, C₂₋₆ alkoxycarbonyl, C₂₋₆ alkoxycarbonyl(C₁₋₆)alkyl, a carboxylic acid isostere or prodrug moiety Ω, —(C₁₋₆)alkyl-Ω, aminocarbonyl, C₁₋₆ alkylaminocarbonyl, di(C₁₋₆)alkylaminocarbonyl, aminosulphonyl or di(C₁₋₆)alkylamino-sulphonyl;
R³² represents hydrogen, halogen, cyano, hydroxy, hydroxy(C₁₋₆)alkyl, C₁₋₆ alkylsulphonyl, formyl, C₂₋₆ alkylcarbonyl, carboxy, carboxy(C₁₋₆)alkyl, C₂₋₆ alkoxycarbonyl, C₂₋₆ alkoxycarbonyl(C₁₋₆)alkyl, aminosulphonyl, (C₁₋₆)alkyl-sulphoximinyl, [(C₁₋₆)alkyl][N—(C₁₋₆)alkyl]sulphoximinyl, a carboxylic acid isostere or prodrug moiety Ω, or —(C₁₋₆)alkyl-Ω;
R³³ represents hydrogen, halogen, C₁₋₆ alkyl, trifluoromethyl, hydroxy, hydroxy-(C₁₋₆)alkyl, C₁₋₆ alkoxy, amino or carboxy;
R³⁴ represents hydrogen, halogen, halo(C₁₋₆)alkyl, hydroxy, C₁₋₆ alkoxy, C₁₋₆ alkylthio, C₁₋₆ alkylsulphinyl, C₁₋₆ alkylsulphonyl, amino, C₁₋₆ alkylamino, di(C₁₋₆)alkyl-amino, (C₂₋₆)alkylcarbonylamino, (C₂₋₆)alkylcarbonylamino(C₁₋₆)alkyl, (C₁₋₆)alkyl-sulphonylamino or (C₁₋₆)alkylsulphonylamino(C₁₋₆)alkyl.

8. The compound as claimed in claim 4 represented by formula (IIC-2) or (IID-2) or an N-oxide thereof, or a pharmaceutically acceptable salt thereof: 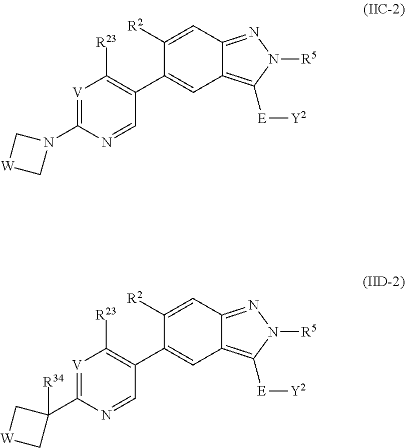
wherein,
E represents a covalent bond; or E represents —O—, —S—, —S(O)—, —S(O)₂— or —N(R⁶)—; or E represents an optionally substituted straight or branched C₁₋₄ alkylene chain;
V represents C—R²² or N;
Y² represents a group of formula (Ya), (Yb), (Yc), (Yd), (Ye) or (Yf): 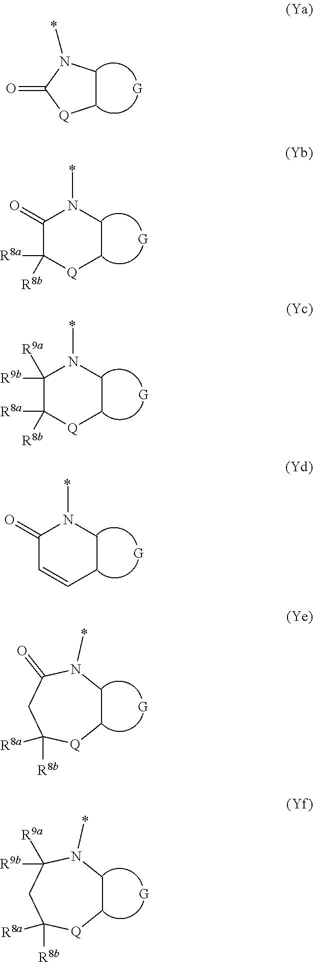
the asterisk (*) represents the point of attachment to the remainder of the molecule;
R² represents hydrogen, halogen, cyano, nitro, hydroxy, trifluoromethyl trifluoromethoxy, —OR^{a}, —SR^{a}, —SOR^{a}, —SO₂R^{a}, —SF₅, —NR^{b}R^{c}, —NR^{c}COR^{d}, —NR^{c}CO₂R^{d}, —NHCONR^{b}R^{c}, —NR^{c}SO₂R^{e}, —N(SO₂R^{e})₂, —NHSO₂NR^{b}R^{c}, —COR^{d}, —CO₂R^{d}, —CONR^{b}R^{c}, —CON(OR^{a})R^{b}, —SO₂NR^{b}R^{c} or —SO(NR^{b})R^{d}, or C₁₋₆ alkyl, C₂₋₆ alkenyl, C₂₋₆ alkynyl, C₃₋₇ cycloakyl, C₄₋₇ cycloalkenyl, C₃₋₇ cycloakyl(C₁₋₆)alkyl, aryl, aryl(C₁₋₆)alkyl, C₃₋₇ heterocycloalkyl, C₃₋₇ heterocycloalkyl(C₁₋₆)alkyl, C₃₋₇ heterocycloalkenyl, C₄₋₉ heterobicycloalkyl, heteroaryl, heteroaryl(C₁₋₆)alkyl, (C₃₋₇)heterocycloalkyl(C₁₋₆)alkyl-aryl-, heteroaryl(C₃₋₇)heterocycloalkyl, (C₃₋₇)cycloalkyl-heteroaryl-, (C₃₋₇)cycloalkyl(C₁₋₆)alkyl-heteroaryl-, (C₄₋₇)cycloalkenyl-heteroaryl-, (C₄₋₉)bicycloalkyl-heteroaryl-, (C₃₋₇)heterocycloalkyl-heteroaryl-, (C₃₋₇)heterocycloalkyl(C₁₋₆)alkyl-heteroaryl-, (C₃₋₇)heterocycloalkenyl-heteroaryl-, (C₄₋₉)heterobicycloalkyl-heteroaryl- or (C₄₋₉)spiroheterocycloalkyl-heteroaryl-, any of which groups may be optionally substituted by one or more substituents;
R⁵ represents C₁₋₆ alkyl, optionally substituted by fluoro, hydroxy, C₁₋₆ alkoxy, amino, C₁₋₆ alkylamino or di(C₁₋₆)alkylamino;
W represents O, S, S(O), S(O)₂, S(O)(NR⁶), N(R³¹) or C(R³²)(R³³);
R²³ represents hydrogen, C₁₋₆ alkyl, trifluoromethyl or C₁₋₆ alkoxy; and
R³⁴ represents hydrogen, halogen, halo(C₁₋₆)alkyl, hydroxy, C₁₋₆ alkoxy, C₁₋₆ alkylthio, C₁₋₆ alkylsulphinyl, C₁₋₆ alkylsulphonyl, amino, C₁₋₆ alkylamino, di(C₁₋₆)alkyl-amino, (C₂₋₆)alkylcarbonylamino, (C₂₋₆)alkylcarbonylamino(C₁₋₆)alkyl, (C₁₋₆)alkyl-sulphonylamino or (C₁₋₆)alkylsulphonylamino(C₁₋₆)alkyl.

9. The compound as claimed in claim 1 wherein E represents —CH₂— or —CH(OH)—.

10. The compound as claimed in claim 1 as herein specifically disclosed in any one of the Examples.

11. (canceled)

12. (canceled)

13. (canceled)

14. A pharmaceutical composition comprising a compound of formula (I) as defined in claim 1 or an N-oxide thereof, or a pharmaceutically acceptable salt thereof, in association with a pharmaceutically acceptable carrier.

15. The pharmaceutical composition as claimed in claim 14 further comprising an additional pharmaceutically active ingredient.

16. (canceled)

17. (canceled)

18. A method for the treatment and/or prevention of disorders for which the administration of a modulator of TNFα function is indicated which comprises administering to a patient in need of such treatment an effective amount of a compound of formula (I) as defined in claim 1 or an N-oxide thereof, or a pharmaceutically acceptable salt thereof.

19. A method for the treatment and/or prevention of an inflammatory or autoimmune disorder, a neurological or neurodegenerative disorder, pain or a nociceptive disorder, a cardiovascular disorder, a metabolic disorder, an ocular disorder, or an oncological disorder, which comprises administering to a patient in need of such treatment an effective amount of a compound of formula (I) as defined in claim 1 or an N-oxide thereof, or a pharmaceutically acceptable salt thereof.
